(19) United States
(12) Patent Application Publication
(10) Pub. No.: US 2023/0416829 A1
(43) Pub. Date: Dec. 28, 2023

(54) Immunotherapy Response Signature

(71) Applicant: Caris MPI, Inc., Irving, TX (US)

(72) Inventors: Jim Abraham, Southlake, TX (US); David Spetzler, Paradise Valley, AZ (US)

(21) Appl. No.: 18/036,215

(22) PCT Filed: Nov. 10, 2020
(86) PCT No.: PCT/US2021/058741

§ 371 Date: May 10, 2023

Related U.S. Application Data
(60) Provisional application No. 63/112,035, filed on Nov. 10, 2020.
(60) Provisional application No. 63/112,359, filed on Nov. 11, 2020.

(51) Int. Cl. C12Q 1/6886; G16H 10/40; G16H 20/10

(52) U.S. Cl. CPC C12Q 1/6886; G16H 10/40; G16H 20/10; C12Q 2600/158

(57) ABSTRACT

Comprehensive molecular profiling provides a wealth of data concerning the molecular status of patient samples. Such data can be compared to patient response to treatments to identify biomarker signatures that predict response or non-response to such treatments. This approach has been applied to identify biomarker signatures that predict cancer patient benefit from immunotherapy such as checkpoint inhibitor therapy.

### CROSS REFERENCE

This application claims the benefit of U.S. Provisional Patent Application Ser. Nos. 63/112,035, filed on Nov. 10, 2020, and 63/112,359, filed on Nov. 11, 2020. The entire contents of the foregoing are hereby incorporated by reference in their entireties.

### TECHNICAL FIELD

The present disclosure relates to the use of molecular profiling to guide personalized treatment recommendations for various diseases and disorders, including without limitation cancer.

### BACKGROUND

Immunotherapy is the treatment of cancer or other diseases by activating or suppressing the immune system. Immunotherapies designed to elicit or amplify an immune response may referred to as activation immunotherapies or immune activators, whereas immunotherapies that reduce or suppress such response may referred to as suppression immunotherapies or immune suppressors. Checkpoint inhibitor therapy is a form of immunotherapy that targets immune checkpoints, which are key regulators of the immune system that stimulate or inhibit immune response. Tumors may block such checkpoints in order to avoid attack by the immune system. Checkpoint therapy can block these inhibitory checkpoints, thereby restoring immune system function. For reviews, see, e.g., Topalian S L et al, Immune checkpoint blockade: a common denominator approach to cancer therapy. Cancer Cell. 2015 Apr. 13; 27(4):450-61; Postow M A et al., Immune Checkpoint Blockade in Cancer Therapy. J Clin Oncol. 2015 Jun. 10; 33(17):1974-82.

PD1 (programmed death-1, PD-1, PDCD1, CD279) is a transmembrane glycoprotein receptor that is expressed on CD4-/CD8-thymocytes in transition to CD4+/CD8+ stage and on mature T and B cells upon activation. It is also present on activated myeloid lineage cells such as monocytes, dendritic cells and NK cells. In normal tissues, PD-1 signaling in T cells regulates immune responses to diminish damage, and counteracts the development of autoimmunity by promoting tolerance to self-antigens. PD-L1 (programmed cell death 1 ligand 1, PDL1, cluster of differentiation 274, CD274, B7 homolog 1, B7-H1, B7H1) and PD-L2 (programmed cell death 1 ligand 2, PDL2, B7-DC, B7DC, CD273, cluster of differentiation 273) are PD1 ligands. In normal cells the PD1/PDL1 interplay is an immune checkpoint, whereas tumor cell expression of PD-L1 is a mechanism to evade recognition/destruction by the immune system, e.g., tumor-infiltrating T cells (TILs). PD-L1 is constitutively expressed in many human cancers including without limitation melanoma, ovarian cancer, lung cancer, clear cell renal cell carcinoma (CRCC), urothelial carcinoma, HNSCC, and esophageal cancer. Monoclonal antibody therapy that targets the PD-1/PD-L1 pathway may allow T-cells to attack the tumor. CTLA4 (cytotoxic T-lymphocyte-associated protein 4, CTLA-4, CD152) is a protein receptor that functions as an immune checkpoint by downregulating immune responses. CTLA4 is constitutively expressed in regulatory T cells but only upregulated in conventional T cells after activation—a phenomenon which is particularly notable in cancers. Monoclonal antibody therapy that blocks inhibitory effects of CTLA-4 can potentiate effective immune responses against tumor cells.

Several targeted therapies to CTLA4, PD-1, and PD-L1 checkpoint inhibitors have been approved by the United States Food and Drug Administration (FDA) for the treatment of various cancers. These include ipilimumab (anti-CTLA-4, trade name Yervoy, Bristol-Myers Squibb); nivolumab (human monoclonal immunoglobulin G4 antibody targeting PD-1, trade name Opdivo, Bristol-Myers Squibb); pembrolizumab (humanized IgG4 isotype antibody targeting PD-1, trade name Keytruda, Merck); atezolizumab (fully humanized, engineered monoclonal antibody of IgG1 isotype targeting PD-L1, trade name Tecentriq, Genentech/Roche); avelumab (whole monoclonal antibody of isotype IgG1 targeting PD-L1, trade name Bavencio, Merck KGaA and Pfizer Inc.); durvalumab (human immunoglobulin G1 kappa (IgG1κ) monoclonal antibody targeting PD-L1, trade name Imfinzi, AstraZeneca); and cemiplimab (monoclonal antibody to PD-1, trade name Libtayo®, Regeneron Pharmaceuticals, Inc., and Sanofi). In May 2017, pembrolizumab received an accelerated approval from the FDA for use in any unresectable or metastatic solid tumor with DNA mismatch repair deficiencies or a microsatellite instability-high state (or, in the case of colon cancer, tumors that have progressed following chemotherapy). This approval marked the first instance in which the FDA approved marketing of a drug based only on the presence of a genetic marker, with no limitation on the site of the cancer or the kind of tissue in which it originated. Several additional therapies that target immune checkpoint proteins are in development.

Despite these successes, immune checkpoint therapy has not proven to be a panacea for cancer. Although pembrolizumab was approved across tumor types, other immunotherapies have only proven efficacy in certain settings. As one example, nivolumab has been approved for inoperable or metastatic melanoma, metastatic squamous non-small cell lung cancer, and as second-line treatment for renal cell carcinoma, but failed to meet its endpoints in a clinical trial directed towards treating newly diagnosed lung cancer. See, e.g., Marin-Acevedo, et al., Next generation of immune checkpoint inhibitors and beyond. Hematol Oncol 14, 45 (2021). Immune checkpoint therapy is also typically prescribed upon indication from a companion diagnostic (e.g., to confirm expression of the target protein), but it is not always efficacious. For example, the response rate to pembrolizumab may be less than 50% even in patients pre-selected for expression of PD-L1 on at least 50% of tumor cells. See, e.g., Reck, M., et al., Pembrolizumab versus Chemotherapy for PD-L1-Positive Non-Small-Cell Lung Cancer. N Engl J Med 2016; 375:1823-1833. And in some cases, checkpoint inhibitor therapy may exacerbate hyperprogressive disease characterized by acceleration of tumor growth during treatment. See, e.g., Ferrara, R et al., Hyperprogressive Disease in Patients With Advanced Non-Small Cell Lung Cancer Treated With PD-1/PD-L1 Inhibitors or With Single-Agent Chemotherapy. JAMA Oncol. 2018 Nov. 1; 4(11):1543-1552. Moreover, altering immune system checkpoint inhibition can have diverse effects on most organ systems of the body. Take pembrolizumab as an example. Adverse reactions include severe infusion-related reactions, severe lung inflammation (including fatalities), inflammation of endocrine organs that caused inflammation of the pituitary gland of the thyroid (causing both hypothyroidism and hyperthyroidism in different people), and pancreatitis that caused Type 1 diabetes and diabetic ketoacidosis. Some patients require lifelong hormone therapy as a result (e.g. insulin therapy or thyroid hormones). Pembrolizumab therapy has also led to colon inflammation, liver inflammation, and kidney inflammation. More common adverse reactions to pembrolizumab include fatigue (24%), rash (19%), itchiness (pruritus) (17%), diarrhea (12%), nausea (11%) and joint pain (arthralgia) (10%), and between 1% and 10% of people taking pembrolizumab have included anemia, decreased appetite, headache, dizziness, distortion of the sense of taste, dry eye, high blood pressure, abdominal pain, constipation, dry mouth, severe skin reactions, vitiligo, various kinds of acne, dry skin, eczema, muscle pain, pain in a limb, arthritis, weakness, edema, fever, chills, and flu-like symptoms. Similar side effects have been observed for other checkpoint inhibitor therapies. Finally, immune checkpoint therapy can be extremely expensive. Indeed, pembrolizumab was priced at $150,000 per year when it launched in late 2014. Taken together, there is a need to better identify those patients more likely to benefit from immunotherapies for better patient outcomes and to avoid unnecessary adverse events and high costs.

### SUMMARY

Comprehensive molecular profiling provides a wealth of data concerning the molecular status of patient samples. Such data can be compared to patient response to treatments to identify biomarker signatures that predict response or non-response to such treatments. This approach has been applied to identify biomarker signatures that correlate with benefit or lack of benefit of immunotherapies, e.g., checkpoint inhibitor therapies and/or chemotherapy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

Accordingly, provided herein is a method of treating a cancer in a subject, the method comprising: (a) obtaining a biological sample comprising cells and/or cell free materials derived from the cancer in the subject; (b) performing an assay to assess a copy number of chromosome 9 or a portion thereof in the biological sample; and (c) administering a treatment for the cancer to the subject based on the assessment of step (b).

In some embodiments, the assessment in step (b) comprises determining a copy number of chromosome 9p or a portion thereof, wherein optionally the assay comprises at least one of sequencing, hybridization, amplification, next-generation sequencing, whole-genome sequencing (WGS), whole-exome sequencing (WES), whole-transcriptome sequencing (WTS), in situ hybridization (ISH), comparative genomic hybridization (CGH), high-resolution array comparative genomic hybridization (aCGH), microarray-based platforms, and PCR techniques.

In some embodiments, the portion of chromosome 9 comprises chromosome band 9p24 or a portion thereof.

In some embodiments, the portion of chromosome 9 comprises one or more gene located in chromosome band 9p24.

In some embodiments, the one or more gene comprises DDX11L5, WASHC1, MIR1302-9HG, MIR1302-9, FAM138C, PGM5P3-AS1, PGM5P3, LINC01388, FOXD4, CBWD1, LOC105375942, LOC105375943, DOCK8, DOCK8-AS1, LOC105375945, LOC112268042, KANK1, RPL12P25, FAM217AP1, LOC105375947, RNU6-1327P, EIF1P1, LOC107987042, LOC105375949, DMRT1, DMRT3, RNU6-1073P, DMRT2, H3P29, LINC01230, RPS27AP14, LOC102723803, RNA5SP279, LOC105375951, LOC105375953, LOC105375952, SMARCA2, RNU2-25P, LOC107987043, RN7SL592P, LOC105375955, LOC101930053, LOC105375956, LOC101930048, VLDLR-AS1, VLDLR, LOC105375957, KCNV2, PUM3, GPS2P1, ATP5PDP2, CARM1P1, LINC01231, LOC105375959, RFX3, RFX3-AS1, LOC105375962, GLIS3, GLIS3-AS1, LOC105375964, LOC107986989, RNU6-694P, SLC1A1, SPATA6L, RPS6P11, PLPP6, CDC37L1-DT, CDC37L1, AK3, ECM1P1, RPS5P6, RCL1, KLF4P1, MIR101-2, HNRNPA1P41, JAK2, INSL6, CSNK1G2P1, PDSS1P1, MTND6P5, MTND5P36, MTND1P11, MTND2P36, MTCO1P11, MTCO2P11, MTATP6P11, MTCO3P11, MTND3P14, MTND4LP6, MTND4P14, MTND5P14, TCF3P1, LOC107987044, IGHEP2, INSL4, RLN2, HMGN2P31, RLN1, PLGRKT, RNF152P1, CD274 (PD-L1), PDCD1LG2 (PD-L2), RIC1, ERMP1, AK4P4, KIAA2026, MLANA, MIR4665, RANBP6, GTF3AP1, IL33, LOC107987046, SELENOTP1, TPD52L3, UHRF2, GLDC, RN7SL25P, RPL23AP57, RN7SL123P, RNF2P1, RPL35AP20, LINC02851, KDM4C, PRELID3BP11, SNRPEP2, ACTG1P14, LOC105375969, LOC105375970, LOC102723994, RPL4P5, PPIAP33, DMAC1, LOC105375971, PTPRD, RPL18AP11, RNU7-185P, PTPRD-AS1, or any useful combination thereof.

In some embodiments, the one or more gene comprises PD-L1, JAK2, or PD-L1 and JAK2, or wherein the one or more gene consists of PD-L1, JAK2, or PD-L1 and JAK2.

In some embodiments, the method described herein further comprises predicting whether the subject will benefit or not benefit from administration of an immunotherapy.

In some embodiments, loss of copy number of chromosome 9 or the portion thereof indicates lack of benefit of the immunotherapy.

In some embodiments, the absence of loss of copy number of chromosome 9 or the portion thereof indicates potential response to the immunotherapy.

In some embodiments, the threshold for loss of copy number is determined using a statistical model, optionally wherein the statistical model is a machine learning model.

In some embodiments, the immunotherapy comprises an immune checkpoint therapy.

In some embodiments, the immune checkpoint therapy comprises at least one of anti-PD-1 therapy, anti-PD-L1 therapy, anti-CTLA-4 therapy, ipilimumab, nivolumab, pembrolizumab, atezolizumab, avelumab, durvalumab, cemiplimab, and any combination thereof.

In some embodiments, the subject has not previously been treated with an immunotherapy or the immunotherapy.

In some embodiments, the cancer comprises a metastatic cancer, a recurrent cancer, or a combination thereof.

In some embodiments, the subject has not previously been treated for the cancer.

In some embodiments, the subject has a loss of copy number of chromosome 9 or the portion thereof and wherein the administered treatment for the cancer is a treatment that is different from the immunotherapy.

In some embodiments, the administered treatment for the cancer is a chemotherapy or a combination of immunotherapy and chemotherapy.

In some embodiments, the subject does not have a loss of copy number of chromosome 9 or the portion thereof and wherein the administered treatment of the cancer is the immunotherapy.

In some embodiments, progression free survival (PFS), disease free survival (DFS), or lifespan is extended by the administration of the treatment.

In some embodiments, the biological sample comprises formalin-fixed paraffin-embedded (FFPE) tissue, fixed tissue, a core needle biopsy, a fine needle aspirate, unstained slides, fresh frozen (FF) tissue, formalin samples, tissue comprised in a solution that preserves nucleic acid or protein molecules, a fresh sample, a malignant fluid, a bodily fluid, a tumor sample, a tissue sample, or any combination thereof.

In some embodiments, the cells and/or cell free materials derived from the cancer are from a solid tumor.

In some embodiments, the biological sample comprises a bodily fluid, and wherein optionally the material derived from cancer cells comprises cell free nucleic acids.

In some embodiments, the bodily fluid comprises a malignant fluid, a pleural fluid, a peritoneal fluid, or any combination thereof.

In some embodiments, the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid, pre-ejaculatory fluid, female ejaculate, sweat, fecal matter, tears, cyst fluid, pleural fluid, peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyst cavity fluid, or umbilical cord blood.

In some embodiments, the cancer comprises an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilms' tumor.

In some embodiments, the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumor (GIST), glioblastoma, head and neck squamous carcinoma, leukemia, liver hepatocellular carcinoma, low grade glioma, lung bronchioloalveolar carcinoma (BAC), non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non-epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma.

In some embodiments, the cancer comprises a head and neck cancer, neuroendocrine cancer, lung cancer, liver cancer, ovarian cancer, or sarcoma.

In some embodiments, the cancer comprises breast cancer, bladder cancer, cervical cancer, colon cancer, head and neck cancer, Hodgkin lymphoma, liver cancer, lung cancer, renal cell cancer, melanoma, stomach cancer, rectal cancer, or any solid tumor that exhibits DNA replication errors, e.g., mutations, insertions, deletions, mismatch repair deficiency (MMRd), microsatellite instability (MSI-H), high tumor mutational burden (TMB), copy number variations (CNV).

In some embodiments, the cancer comprises a head and neck cancer or a lung cancer.

Also provided herein is a method of selecting a treatment for a subject who has a cancer, the method comprising: (a) obtaining a biological sample comprising cells and/or cell free material derived from the cancer in the subject; (b) performing an assay to assess a copy number of chromosome 9 or a portion thereof in the biological sample, wherein optionally the assay comprises at least one of sequencing, hybridization, amplification, next-generation sequencing, whole-genome sequencing (WGS), whole-exome sequencing (WES), whole-transcriptome sequencing (WTS), in situ hybridization (ISH), comparative genomic hybridization (CGH), high-resolution array comparative genomic hybridization (aCGH), microarray-based platforms, and PCR techniques; and (c) selecting a treatment for the cancer to the subject based on the copy number of chromosome 9 or the portion thereof in (b).

In some embodiments, the portion of chromosome 9 comprises arm 9p, band 9p24, one or more gene located at 9p24, the PD-L1 gene, the JAK2 gene, the PD-L1 and JAK2 genes, or any useful combination thereof, optionally wherein the one or more gene comprises DDX11L5, WASHC1, MIR1302-9HG, MIR1302-9, FAM138C, PGM5P3-AS1, PGM5P3, LINC01388, FOXD4, CBWD1, LOC105375942, LOC105375943, DOCK8, DOCK8-AS1, LOC105375945, LOC112268042, KANK1, RPL12P25, FAM217AP1, LOC105375947, RNU6-1327P, EIF1P1, LOC107987042, LOC105375949, DMRT1, DMRT3, RNU6-1073P, DMRT2, H3P29, LINC01230, RPS27AP14, LOC102723803, RNA5SP279, LOC105375951, LOC105375953, LOC105375952, SMARCA2, RNU2-25P, LOC107987043, RN7SL592P, LOC105375955, LOC101930053, LOC105375956, LOC101930048, VLDLR-AS1, VLDLR, LOC105375957, KCNV2, PUM3, GPS2P1, ATP5PDP2, CARM1P1, LINC01231, LOC105375959, RFX3, RFX3-AS1, LOC105375962, GLIS3, GLIS3-AS1, LOC105375964, LOC107986989, RNU6-694P, SLC1A1, SPATA6L, RPS6P11, PLPP6, CDC37L1-DT, CDC37L1, AK3, ECM1P1, RPS5P6, RCL1, KLF4P1, MIR101-2, HNRNPA1P41, JAK2, INSL6, CSNK1G2P1, PDSS1P1, MTND6P5, MTND5P36, MTND1P11, MTND2P36, MTCO1P11, MTCO2P11, MTATP6P11, MTCO3P11, MTND3P14, MTND4LP6, MTND4P14, MTND5P14, TCF3P1, LOC107987044, IGHEP2, INSL4, RLN2, HMGN2P31, RLN1, PLGRKT, RNF152P1, CD274 (PD-L1), PDCD1LG2 (PD-L2), RIC1, ERMP1, AK4P4, KIAA2026, MLANA, MIR4665, RANBP6, GTF3AP1, IL33, LOC107987046, SELENOTP1, TPD52L3, UHRF2, GLDC, RN7SL25P, RPL23AP57, RN7SL123P, RNF2P1, RPL35AP20, LINC02851, KDM4C, PRELID3BP11, SNRPEP2, ACTG1P14, LOC105375969, LOC105375970, LOC102723994, RPL4P5, PPIAP33, DMAC1, LOC105375971, PTPRD, RPL18AP11, RNU7-185P, PTPRD-AS1, or any useful combination thereof.

In some embodiments, the method described herein further comprises preparing a molecular profile for the subject based on the copy number of chromosome 9 or the portion thereof.

In some embodiments, the treatment comprises a checkpoint inhibitor therapy, e.g., anti-PD-1 therapy, anti-PD-L1 therapy, anti-CTLA-4 therapy, nivolumab, pembrolizumab, ipilimumab, atezolizumab, avelumab, durvalumab, or cemiplimab, a chemotherapy, or any useful combination thereof, based on the copy number.

In some embodiments, the method described herein further comprises administering the checkpoint inhibitor therapy to the subject when the subject is predicted to benefit from the therapy, and/or administering chemotherapy or chemotherapy in addition to the checkpoint inhibitor therapy when the subject is predicted to lack benefit from the therapy.

In some embodiments, the cancer comprises a head and neck cancer, neuroendocrine cancer, lung cancer, liver cancer, ovarian cancer, or sarcoma; or breast cancer, bladder cancer, cervical cancer, colon cancer, head and neck cancer, Hodgkin lymphoma, liver cancer, lung cancer, renal cell cancer, melanoma, stomach cancer, rectal cancer, or any solid tumor that exhibits DNA replication errors, e.g., mutations, insertions, deletions, mismatch repair deficiency (MMRd), microsatellite instability (MSI-H), high tumor mutational burden (TMB), copy number variations (CNV).

In some embodiments, the methods described herein include detecting a copy number variation, wherein loss of one or both copies, e.g., a copy number below two, e.g., one (1) or zero (0) copy, is detected in a sample from a subject, then the subject who has 0 or 1 copy of chromosome 9 or a portion thereof, e.g., as described herein, is optionally identified as a subject who is not likely to respond to immunotherapy and a treatment other than immunotherapy (e.g., radiotherapy, chemotherapy, or surgical resection) should be selected and/or administered. Subjects who are identified as having a normal copy number (e.g., 2), or a gain of copy number (more than 2), can be optionally identified as a subject who is likely to respond to immunotherapy, and a treatment comprising immunotherapy (and optionally another therapy (e.g., radiotherapy, chemotherapy, or surgical resection) should be selected and optionally administered.

Also provided herein is a method of generating a molecular profiling report comprising preparing a report summarizing results of performing the method according to any of the methods described herein.

In some embodiments, the report comprises any identified treatment of likely benefit and/or lack of benefit according to any of the methods described herein.

In some embodiments, the report is computer generated; is a printed report or a computer file; or is accessible via a web portal.

Also provided herein is a system comprising one or more computers and one or more storage media storing instructions that, when executed by the one or more computers, cause the one or more computers to perform operations in order to carry out any of the methods described herein.

Also provided herein is a system for identifying a treatment for a cancer in a subject, the system comprising: (a) at least one host server; (b) at least one user interface for accessing the at least one host server to access and input data; (c) at least one processor for processing the inputted data; (d) at least one memory coupled to the processor for storing the processed data and instructions for: (1) accessing results of analyzing the biological sample according to any of the methods described herein; and (2) determining likely benefit or lack of benefit of an immunotherapy according to any of the methods described herein; and (e) at least one display for displaying the likely benefit or lack of benefit of the immunotherapy for treating the cancer.

In some embodiments, the at least one display comprises a report comprising the results of analyzing the biological sample and the predicted likely benefit or lack of benefit for treatment of the cancer.

### DESCRIPTION OF DRAWINGS

FIG. 1 illustrates a block diagram of an exemplary embodiment of a system for determining individualized medical intervention for cancer that utilizes molecular profiling of a patient's biological specimen as described herein.

FIGS. 2A-C are flowcharts of exemplary embodiments of (A) a method for determining individualized medical intervention for cancer that utilizes molecular profiling of a patient's biological specimen, (B) a method for identifying signatures or molecular profiles that can be used to predict benefit from therapy, and (C) an alternate version of (B).

FIGS. 3A-3V show prediction of response of various cancers to immunotherapy.

### DETAILED DESCRIPTION

Described herein are methods and systems for characterizing various phenotypes of biological systems, organisms, cells, samples, or the like, and methods for treating cancers, by using molecular profiling, including systems, methods, apparatuses, and computer programs, to characterize such phenotypes. The term “phenotype” as used herein can mean any trait or characteristic that can be identified in part or in whole by using the systems and/or methods provided herein. In some implementations, the systems can include one or more computer programs on one or more computers in one or more locations, e.g., configured for use in a method described herein.

Phenotypes to be characterized can be any phenotype of interest, including without limitation a tissue, anatomical origin, medical condition, ailment, disease, disorder, or useful combinations thereof. A phenotype can be any observable characteristic or trait of, such as a disease or condition, a stage of a disease or condition, susceptibility to a disease or condition, prognosis of a disease stage or condition, a physiological state, or response/potential response (or lack thereof) to interventions such as therapeutics. A phenotype can result from a subject's genetic makeup as well as the influence of environmental factors and the interactions between the two, as well as from epigenetic modifications to nucleic acid sequences.

In various embodiments, a phenotype in a subject is characterized by obtaining a biological sample from a subject and analyzing the sample using the systems and/or methods provided herein. For example, characterizing a phenotype for a subject or individual can include detecting a disease or condition (including pre-symptomatic early stage detection), determining a prognosis, diagnosis, or theranosis of a disease or condition, or determining the stage or progression of a disease or condition. Characterizing a phenotype can include identifying appropriate treatments or treatment efficacy for specific diseases, conditions, disease stages and condition stages, predictions and likelihood analysis of disease progression, particularly disease recurrence, metastatic spread or disease relapse. A phenotype can also be a clinically distinct type or subtype of a condition or disease, such as a cancer or tumor. Phenotype determination can also be a determination of a physiological condition, or an assessment of organ distress or organ rejection, such as post-transplantation. The compositions and methods described herein allow assessment of a subject on an individual basis, which can provide benefits of more efficient and economical decisions in treatment.

Theranostics includes diagnostic testing that provides the ability to affect therapy or treatment of a medical condition such as a disease or disease state. Theranostics testing provides a theranosis in a similar manner that diagnostics or prognostic testing provides a diagnosis or prognosis, respectively. As used herein, theranostics encompasses any desired form of therapy related testing, including predictive medicine, personalized medicine, precision medicine, integrated medicine, pharmacodiagnostics and Dx/Rx partnering. Therapy related tests can be used to predict and assess drug response in individual subjects, thereby providing personalized medical recommendations. Predicting a likelihood of response can be determining whether a subject is a likely responder or a likely non-responder to a candidate therapeutic agent, e.g., before the subject has been exposed or otherwise treated with the treatment. Assessing a therapeutic response can be monitoring a response to a treatment, e.g., monitoring the subject's improvement or lack thereof over a time course after initiating the treatment. Therapy related tests are useful to select a subject for treatment who is particularly likely to benefit or lack benefit from the treatment or to provide an early and objective indication of treatment efficacy in an individual subject. Characterization using the systems and methods provided herein may indicate that treatment should be altered to select a more promising treatment, thereby avoiding the expense of delaying beneficial treatment and avoiding the financial and morbidity costs of less efficacious or ineffective treatment(s).

In various embodiments, a theranosis comprises predicting a treatment efficacy or lack thereof, classifying a patient as a responder or non-responder to treatment. A predicted “responder” can refer to a patient likely to receive a benefit from a treatment whereas a predicted “non-responder” can be a patient unlikely to receive a benefit from the treatment. Unless specified otherwise, a benefit can be any clinical benefit of interest, including without limitation cure in whole or in part, remission, or any improvement, reduction or decline in progression of the condition or symptoms. The theranosis can be directed to any appropriate treatment, e.g., the treatment may comprise at least one of chemotherapy, immunotherapy, targeted cancer therapy, a monoclonal antibody, small molecule, or any useful combinations thereof.

The phenotype can comprise detecting the presence of or likelihood of developing a tumor, neoplasm, or cancer, or characterizing the tumor, neoplasm, or cancer (e.g., stage, grade, aggressiveness, likelihood of metastasis or recurrence, etc). In some embodiments, the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non-epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. The systems and methods herein can be used to characterize these and other cancers. Thus, characterizing a phenotype can be providing a diagnosis, prognosis or theranosis of one of the cancers disclosed herein.

In various embodiments, the phenotype comprises a tissue or anatomical origin. For example, the tissue can be muscle, epithelial, connective tissue, nervous tissue, or any combination thereof. For example, the anatomical origin can be the stomach, liver, small intestine, large intestine, rectum, anus, lungs, nose, bronchi, kidneys, urinary bladder, urethra, pituitary gland, pineal gland, adrenal gland, thyroid, pancreas, parathyroid, prostate, heart, blood vessels, lymph node, bone marrow, thymus, spleen, skin, tongue, nose, eyes, ears, teeth, uterus, vagina, testis, penis, ovaries, breast, mammary glands, brain, spinal cord, nerve, bone, ligament, tendon, or any combination thereof. Additional non-limiting examples of phenotypes of interest include clinical characteristics, such as a stage or grade of a tumor, or the tumor's origin, e.g., the tissue origin.

In various embodiments, phenotypes are determined by analyzing a biological sample obtained from a subject. A subject (individual, patient, or the like) can include, but is not limited to, mammals such as bovine, avian, canine, equine, feline, ovine, porcine, or primate animals (including humans and non-human primates). In preferred embodiments, the subject is a human subject. The subject can have a pre-existing disease or condition, including without limitation cancer. Alternatively, the subject may not have any known pre-existing condition. The subject may also be non-responsive to an existing or past treatment, such as a treatment for cancer.

Systems

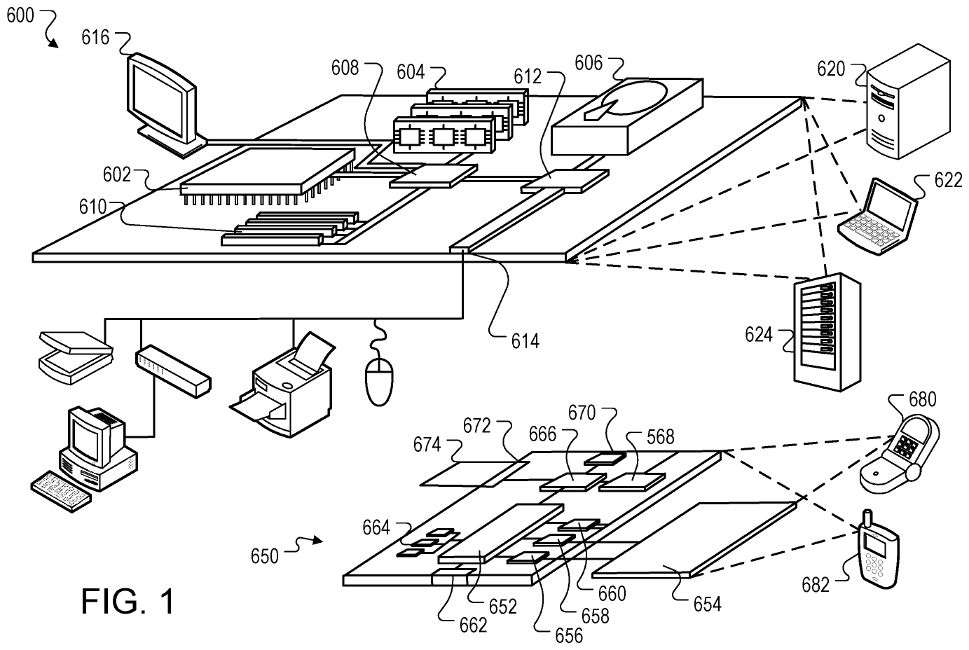

FIG. 1 is a block diagram of system components that can be used to implement a system for selecting treatment for cancer.

Computing device 600 is intended to represent various forms of digital computers, such as laptops, desktops, workstations, personal digital assistants, servers, blade servers, mainframes, and other appropriate computers. Computing device 650 is intended to represent various forms of mobile devices, such as personal digital assistants, cellular telephones, smartphones, and other similar computing devices. Additionally, computing device 600 or 650 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the systems and methods described and/or claimed in this document.

Computing device 600 includes a processor 602, memory 604, a storage device 608, a high-speed interface 608 connecting to memory 604 and high-speed expansion ports 610, and a low speed interface 612 connecting to low speed bus 614 and storage device 608. Each of the components 602, 604, 608, 608, 610, and 612, are interconnected using various busses, and can be mounted on a common motherboard or in other manners as appropriate. The processor 602 can process instructions for execution within the computing device 600, including instructions stored in the memory 604 or on the storage device 608 to display graphical information for a GUI on an external input/output device, such as display 616 coupled to high speed interface 608. In other implementations, multiple processors and/or multiple buses can be used, as appropriate, along with multiple memories and types of memory. Also, multiple computing devices 600 can be connected, with each device providing portions of the necessary operations, e.g., as a server bank, a group of blade servers, or a multi-processor system.

The memory 604 stores information within the computing device 600. In one implementation, the memory 604 is a volatile memory unit or units. In another implementation, the memory 604 is a non-volatile memory unit or units. The memory 604 can also be another form of computer-readable medium, such as a magnetic or optical disk.

The storage device 608 is capable of providing mass storage for the computing device 600. In one implementation, the storage device 608 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 604, the storage device 608, or memory on processor 602.

The high speed controller 608 manages bandwidth-intensive operations for the computing device 600, while the low speed controller 612 manages lower bandwidth intensive operations. Such allocation of functions is exemplary only. In one implementation, the high-speed controller 608 is coupled to memory 604, display 616, e.g., through a graphics processor or accelerator, and to high-speed expansion ports 610, which can accept various expansion cards (not shown). In the implementation, low-speed controller 612 is coupled to storage device 608 and low-speed expansion port 614. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 600 can be implemented in a number of different forms, as shown in the figure. For example, it can be implemented as a standard server 620, or multiple times in a group of such servers. It can also be implemented as part of a rack server system 624. In addition, it can be implemented in a personal computer such as a laptop computer 622. Alternatively, components from computing device 600 can be combined with other components in a mobile device (not shown), such as device 650. Each of such devices can contain one or more of computing device 600, 650, and an entire system can be made up of multiple computing devices 600, 650 communicating with each other.

The computing device 600 can be implemented in a number of different forms, as shown in the figure. For example, it can be implemented as a standard server 620, or multiple times in a group of such servers. It can also be implemented as part of a rack server system 624. In addition, it can be implemented in a personal computer such as a laptop computer 622. Alternatively, components from computing device 600 can be combined with other components in a mobile device (not shown), such as device 650. Each of such devices can contain one or more of computing device 600, 650, and an entire system can be made up of multiple computing devices 600, 650 communicating with each other.

Computing device 650 includes a processor 652, memory 664, and an input/output device such as a display 654, a communication interface 666, and a transceiver 668, among other components. The device 650 can also be provided with a storage device, such as a micro-drive or other device, to provide additional storage. Each of the components 650, 652, 664, 654, 666, and 668, are interconnected using various buses, and several of the components can be mounted on a common motherboard or in other manners as appropriate.

The processor 652 can execute instructions within the computing device 650, including instructions stored in the memory 664. The processor can be implemented as a chipset of chips that include separate and multiple analog and digital processors. Additionally, the processor can be implemented using any of a number of architectures. For example, the processor 610 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 650, such as control of user interfaces, applications run by device 650, and wireless communication by device 650.

Processor 652 can communicate with a user through control interface 658 and display interface 656 coupled to a display 654. The display 654 can be, for example, a TFT (Thin-Film-Transistor Liquid Crystal Display) display or an OLED (Organic Light Emitting Diode) display, or other appropriate display technology. The display interface 656 can comprise appropriate circuitry for driving the display 654 to present graphical and other information to a user. The control interface 658 can receive commands from a user and convert them for submission to the processor 652. In addition, an external interface 662 can be provide in communication with processor 652, so as to enable near area communication of device 650 with other devices. External interface 662 can provide, for example, for wired communication in some implementations, or for wireless communication in other implementations, and multiple interfaces can also be used.

The memory 664 stores information within the computing device 650. The memory 664 can be implemented as one or more of a computer-readable medium or media, a volatile memory unit or units, or a non-volatile memory unit or units. Expansion memory 674 can also be provided and connected to device 650 through expansion interface 672, which can include, for example, a SIMM (Single In Line Memory Module) card interface. Such expansion memory 674 can provide extra storage space for device 650, or can also store applications or other information for device 650. Specifically, expansion memory 674 can include instructions to carry out or supplement the processes described above, and can include secure information also. Thus, for example, expansion memory 674 can be provide as a security module for device 650, and can be programmed with instructions that permit secure use of device 650. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 664, expansion memory 674, or memory on processor 652 that can be received, for example, over transceiver 668 or external interface 662.

Device 650 can communicate wirelessly through communication interface 666, which can include digital signal processing circuitry where necessary. Communication interface 666 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 668. In addition, short-range communication can occur, such as using a Bluetooth, Wi-Fi, or other such transceiver (not shown). In addition, GPS (Global Positioning System) receiver module 670 can provide additional navigation- and location-related wireless data to device 650, which can be used as appropriate by applications running on device 650.

Device 650 can also communicate audibly using audio codec 660, which can receive spoken information from a user and convert it to usable digital information. Audio codec 660 can likewise generate audible sound for a user, such as through a speaker, e.g., in a handset of device 650. Such sound can include sound from voice telephone calls, can include recorded sound, e.g., voice messages, music files, etc. and can also include sound generated by applications operating on device 650.

The computing device 650 can be implemented in a number of different forms, as shown in the figure. For example, it can be implemented as a cellular telephone 680. It can also be implemented as part of a smartphone 682, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components.

The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

Molecular Profiling

The molecular profiling approach provides a method for selecting a candidate treatment for an individual that could favorably change the clinical course for the individual with a condition or disease, such as cancer. The molecular profiling approach provides clinical benefit for individuals, such as identifying therapeutic regimens that provide a longer progression free survival (PFS), longer disease free survival (DFS), longer overall survival (OS) or extended lifespan. Methods and systems as described herein are directed to molecular profiling of cancer on an individual basis that can identify optimal therapeutic regimens. Molecular profiling provides a personalized approach to selecting candidate treatments that are likely to benefit a cancer. The molecular profiling methods described herein can be used to guide treatment in any desired setting, including without limitation the front-line/standard of care setting, or for patients with poor prognosis, such as those with metastatic disease or those whose cancer has progressed on standard front line therapies, or whose cancer has progressed on previous chemotherapeutic or hormonal regimens.

The systems and methods provided herein may be used to classify patients as more or less likely to benefit or respond to various treatments. Unless otherwise noted, the terms “response” or “non-response,” as used herein, refer to any appropriate indication that a treatment provides a benefit to a patient (a “responder” or “benefiter”) or has a lack of benefit to the patient (a “non-responder” or “non-benefiter”). Such an indication may be determined using accepted clinical response criteria such as the standard Response Evaluation Criteria in Solid Tumors (RECIST) criteria, or other useful patient response criteria such as progression free survival (PFS), time to progression (TTP), disease free survival (DFS), time-to-next treatment (TNT, TTNT), tumor shrinkage or disappearance, or the like. RECIST is a set of rules published by an international consortium that define when tumors improve (“respond”), stay the same (“stabilize”), or worsen (“progress”) during treatment of a cancer patient. As used herein and unless otherwise noted, a patient “benefit” from a treatment may refer to any appropriate measure of improvement, including without limitation a RECIST response or longer PFS/TTP/DFS/TNT/TTNT, whereas “lack of benefit” from a treatment may refer to any appropriate measure of worsening disease during treatment. Generally disease stabilization is considered a benefit, although in certain circumstances, if so noted herein, stabilization may be considered a lack of benefit. A predicted or indicated benefit may be described as “indeterminate” if there is not an acceptable level of prediction of benefit or lack of benefit. In some cases, benefit is considered indeterminate if it cannot be calculated, e.g., due to lack of necessary data.

Personalized medicine based on pharmacogenetic insights, such as those provided by molecular profiling as described herein, is increasingly taken for granted by some practitioners and the lay press, but forms the basis of hope for improved cancer therapy. However, molecular profiling as taught herein represents a fundamental departure from the traditional approach to oncologic therapy where for the most part, patients are grouped together and treated with approaches that are based on findings from light microscopy and disease stage. Traditionally, differential response to a particular therapeutic strategy has only been determined after the treatment was given, i.e., aposteriori. The “standard” approach to disease treatment relies on what is generally true about a given cancer diagnosis and treatment response has been vetted by randomized phase III clinical trials and forms the “standard of care” in medical practice. The results of these trials have been codified in consensus statements by guidelines organizations such as the National Comprehensive Cancer Network and The American Society of Clinical Oncology. The NCCN Compendium™ contains authoritative, scientifically derived information designed to support decision-making about the appropriate use of drugs and biologics in patients with cancer. The NCCN Compendium™ is recognized by the Centers for Medicare and Medicaid Services (CMS) and United Healthcare as an authoritative reference for oncology coverage policy. On-compendium treatments are those recommended by such guides. The biostatistical methods used to validate the results of clinical trials rely on minimizing differences between patients, and are based on declaring the likelihood of error that one approach is better than another for a patient group defined only by light microscopy and stage, not by individual differences in tumors. The molecular profiling methods described herein exploit such individual differences. The methods can provide candidate treatments that can be then selected by a physician for treating a patient.

Molecular profiling can be used to provide a comprehensive view of the biological state of a sample. In an embodiment, molecular profiling is used for whole tumor profiling. Accordingly, a number of molecular approaches are used to assess the state of a tumor. The whole tumor profiling can be used for selecting a candidate treatment for a tumor. Molecular profiling can be used to select candidate therapeutics on any sample for any stage of a disease. In embodiment, the methods as described herein are used to profile a newly diagnosed cancer. The candidate treatments indicated by the molecular profiling can be used to select a therapy for treating the newly diagnosed cancer. In other embodiments, the methods as described herein are used to profile a cancer that has already been treated, e.g., with one or more standard-of-care therapy. In embodiments, the cancer is refractory to the prior treatment/s. For example, the cancer may be refractory to the standard of care treatments for the cancer. The cancer can be a metastatic cancer or other recurrent cancer. The treatments can be on-compendium or off-compendium treatments.

Molecular profiling can be performed by any known means for detecting a molecule in a biological sample. Molecular profiling comprises methods that include but are not limited to, nucleic acid sequencing, such as a DNA sequencing or RNA sequencing; immunohistochemistry (IHC); in situ hybridization (ISH); fluorescent in situ hybridization (FISH); chromogenic in situ hybridization (CISH); PCR amplification (e.g., qPCR or RT-PCR); various types of microarray (mRNA expression arrays, low density arrays, protein arrays, etc); various types of sequencing (Sanger, pyrosequencing, etc); comparative genomic hybridization (CGH); high throughput or next generation sequencing (NGS); Northern blot; Southern blot; immunoassay; and any other appropriate technique to assay the presence or quantity of a biological molecule of interest. In various embodiments, any one or more of these methods can be used concurrently or subsequent to each other for assessing target genes disclosed herein.

Molecular profiling of individual samples is used to select one or more candidate treatments for a disorder in a subject, e.g., by identifying targets for drugs that may be effective for a given cancer. For example, the candidate treatment can be a treatment known to have an effect on cells that differentially express genes as identified by molecular profiling techniques, an experimental drug, a government or regulatory approved drug or any combination of such drugs, which may have been studied and approved for a particular indication that is the same as or different from the indication of the subject from whom a biological sample is obtain and molecularly profiled.

When multiple biomarker targets are revealed by assessing target genes by molecular profiling, one or more decision rules can be put in place to prioritize the selection of certain therapeutic agent for treatment of an individual on a personalized basis. Rules as described herein aide prioritizing treatment, e.g., direct results of molecular profiling, anticipated efficacy of therapeutic agent, prior history with the same or other treatments, expected side effects, availability of therapeutic agent, cost of therapeutic agent, drug-drug interactions, and other factors considered by a treating physician. Based on the recommended and prioritized therapeutic agent targets, a physician can decide on the course of treatment for a particular individual. Accordingly, molecular profiling methods and systems as described herein can select candidate treatments based on individual characteristics of diseased cells, e.g., tumor cells, and other personalized factors in a subject in need of treatment, as opposed to relying on a traditional one-size fits all approach that is conventionally used to treat individuals suffering from a disease, especially cancer. In some cases, the recommended treatments are those not typically used to treat the disease or disorder inflicting the subject. In some cases, the recommended treatments are used after standard-of-care therapies are no longer providing adequate efficacy.

The treating physician can use the results of the molecular profiling methods to optimize a treatment regimen for a patient. The candidate treatment identified by the methods as described herein can be used to treat a patient; however, such treatment is not required of the methods. Indeed, the analysis of molecular profiling results and identification of candidate treatments based on those results can be automated and does not require physician involvement.

Biological Entities

Nucleic acids include deoxyribonucleotides or ribonucleotides and polymers thereof in either single- or double-stranded form, or complements thereof. Nucleic acids can contain known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Nucleic acid sequence can encompass conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell Probes 8:91-98 (1994)). The term nucleic acid can be used interchangeably with gene, cDNA, mRNA, oligonucleotide, and polynucleotide.

A particular nucleic acid sequence may implicitly encompass the particular sequence and “splice variants” and nucleic acid sequences encoding truncated forms. Similarly, a particular protein encoded by a nucleic acid can encompass any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or created using recombinant techniques.

The terms “genetic variant” and “nucleotide variant” are used herein interchangeably to refer to changes or alterations to the reference human gene or cDNA sequence at a particular locus, including, but not limited to, nucleotide base deletions, insertions, inversions, and substitutions in the coding and non-coding regions. Deletions may be of a single nucleotide base, a portion or a region of the nucleotide sequence of the gene, or of the entire gene sequence. Insertions may be of one or more nucleotide bases. The genetic variant or nucleotide variant may occur in transcriptional regulatory regions, untranslated regions of mRNA, exons, introns, exon/intron junctions, etc. The genetic variant or nucleotide variant can potentially result in stop codons, frame shifts, deletions of amino acids, altered gene transcript splice forms or altered amino acid sequence.

An allele or gene allele comprises generally a naturally occurring gene having a reference sequence or a gene containing a specific nucleotide variant.

A haplotype refers to a combination of genetic (nucleotide) variants in a region of an mRNA or a genomic DNA on a chromosome found in an individual. Thus, a haplotype includes a number of genetically linked polymorphic variants which are typically inherited together as a unit.

As used herein, the term “amino acid variant” is used to refer to an amino acid change to a reference human protein sequence resulting from genetic variants or nucleotide variants to the reference human gene encoding the reference protein. The term “amino acid variant” is intended to encompass not only single amino acid substitutions, but also amino acid deletions, insertions, and other significant changes of amino acid sequence in the reference protein.

The term “genotype” as used herein means the nucleotide characters at a particular nucleotide variant marker (or locus) in either one allele or both alleles of a gene (or a particular chromosome region). With respect to a particular nucleotide position of a gene of interest, the nucleotide(s) at that locus or equivalent thereof in one or both alleles form the genotype of the gene at that locus. A genotype can be homozygous or heterozygous. Accordingly, “genotyping” means determining the genotype, that is, the nucleotide(s) at a particular gene locus. Genotyping can also be done by determining the amino acid variant at a particular position of a protein which can be used to deduce the corresponding nucleotide variant(s).

The term “locus” refers to a specific position or site in a gene sequence or protein. Thus, there may be one or more contiguous nucleotides in a particular gene locus, or one or more amino acids at a particular locus in a polypeptide. Moreover, a locus may refer to a particular position in a gene where one or more nucleotides have been deleted, inserted, or inverted.

Unless specified otherwise or understood by one of skill in art, the terms “polypeptide,” “protein,” and “peptide” are used interchangeably herein to refer to an amino acid chain in which the amino acid residues are linked by covalent peptide bonds. The amino acid chain can be of any length of at least two amino acids, including full-length proteins. Unless otherwise specified, polypeptide, protein, and peptide also encompass various modified forms thereof, including but not limited to glycosylated forms, phosphorylated forms, etc. A polypeptide, protein or peptide can also be referred to as a gene product.

Lists of gene and gene products that can be assayed by molecular profiling techniques are presented herein. Lists of genes may be presented in the context of molecular profiling techniques that detect a gene product (e.g., an mRNA or protein). One of skill will understand that this implies detection of the gene product of the listed genes. Similarly, lists of gene products may be presented in the context of molecular profiling techniques that detect a gene sequence or copy number. One of skill will understand that this implies detection of the gene corresponding to the gene products, including as an example DNA encoding the gene products. As will be appreciated by those skilled in the art, a “biomarker” or “marker” comprises a gene and/or gene product depending on the context.

The terms “primer”, “probe,” and “oligonucleotide” are used herein interchangeably to refer to a relatively short nucleic acid fragment or sequence. They can comprise DNA, RNA, or a hybrid thereof, or chemically modified analog or derivatives thereof. Typically, they are single-stranded. However, they can also be double-stranded having two complementing strands which can be separated by denaturation. Normally, primers, probes and oligonucleotides have a length of from about 8 nucleotides to about 200 nucleotides, preferably from about 12 nucleotides to about 100 nucleotides, and more preferably about 18 to about 50 nucleotides. They can be labeled with detectable markers or modified using conventional manners for various molecular biological applications.

The term “isolated” when used in reference to nucleic acids (e.g., genomic DNAs, cDNAs, mRNAs, or fragments thereof) is intended to mean that a nucleic acid molecule is present in a form that is substantially separated from other naturally occurring nucleic acids that are normally associated with the molecule. Because a naturally existing chromosome (or a viral equivalent thereof) includes a long nucleic acid sequence, an isolated nucleic acid can be a nucleic acid molecule having only a portion of the nucleic acid sequence in the chromosome but not one or more other portions present on the same chromosome. More specifically, an isolated nucleic acid can include naturally occurring nucleic acid sequences that flank the nucleic acid in the naturally existing chromosome (or a viral equivalent thereof). An isolated nucleic acid can be substantially separated from other naturally occurring nucleic acids that are on a different chromosome of the same organism. An isolated nucleic acid can also be a composition in which the specified nucleic acid molecule is significantly enriched so as to constitute at least 10%, 20%, 30%, 40%, 50%, 60%, 70%, 80%, 90%, 95%, or at least 99% of the total nucleic acids in the composition.

An isolated nucleic acid can be a hybrid nucleic acid having the specified nucleic acid molecule covalently linked to one or more nucleic acid molecules that are not the nucleic acids naturally flanking the specified nucleic acid. For example, an isolated nucleic acid can be in a vector. In addition, the specified nucleic acid may have a nucleotide sequence that is identical to a naturally occurring nucleic acid or a modified form or mutein thereof having one or more mutations such as nucleotide substitution, deletion/insertion, inversion, and the like.

An isolated nucleic acid can be prepared from a recombinant host cell (in which the nucleic acids have been recombinantly amplified and/or expressed), or can be a chemically synthesized nucleic acid having a naturally occurring nucleotide sequence or an artificially modified form thereof.

The term “high stringency hybridization conditions,” when used in connection with nucleic acid hybridization, includes hybridization conducted overnight at 42° C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 0.1×SSC at about 65° C. The term “moderate stringent hybridization conditions,” when used in connection with nucleic acid hybridization, includes hybridization conducted overnight at 37° C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

For the purpose of comparing two different nucleic acid or polypeptide sequences, one sequence (test sequence) may be described to be a specific percentage identical to another sequence (comparison sequence). The percentage identity can be determined by the algorithm of Karlin and Altschul, Proc. Natl. Acad. Sci. USA, 90:5873-5877 (1993), which is incorporated into various BLAST programs. The percentage identity can be determined by the “BLAST 2 Sequences” tool, which is available at the National Center for Biotechnology Information (NCBI) website. See Tatusova and Madden, FEMS Microbiol. Lett., 174(2):247-250 (1999). For pairwise DNA-DNA comparison, the BLASTN program is used with default parameters (e.g., Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP program can be employed using default parameters (e.g., Matrix: BLOSUM62; gap open: 11; gap extension: 1; x_dropoff: 15; expect: 10.0; and wordsize: 3, with filter). Percent identity of two sequences is calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence. When BLAST is used to compare two sequences, it aligns the sequences and yields the percent identity over defined, aligned regions. If the two sequences are aligned across their entire length, the percent identity yielded by the BLAST is the percent identity of the two sequences. If BLAST does not align the two sequences over their entire length, then the number of identical amino acids or nucleotides in the unaligned regions of the test sequence and comparison sequence is considered to be zero and the percent identity is calculated by adding the number of identical amino acids or nucleotides in the aligned regions and dividing that number by the length of the comparison sequence. Various versions of the BLAST programs can be used to compare sequences, e.g., BLAST 2.1.2 or BLAST+2.2.22.

A subject or individual can be any animal which may benefit from the methods described herein, including, e.g., humans and non-human mammals, such as primates, rodents, horses, dogs and cats. Subjects include without limitation a eukaryotic organisms, most preferably a mammal such as a primate, e.g., chimpanzee or human, cow; dog; cat; a rodent, e.g., guinea pig, rat, mouse; rabbit; or a bird; reptile; or fish. Subjects specifically intended for treatment using the methods described herein include humans. A subject may also be referred to herein as an individual or a patient. In the present methods the subject has colorectal cancer, e.g., has been diagnosed with colorectal cancer. Methods for identifying subjects with colorectal cancer are known in the art, e.g., using a biopsy. See, e.g., Fleming et al., J Gastrointest Oncol. 2012 September; 3(3): 153-173; Chang et al., Dis Colon Rectum. 2012; 55(8):831-43.

Treatment of a disease or individual according to the methods described herein is an approach for obtaining beneficial or desired medical results, including clinical results, but not necessarily a cure. For purposes of the methods described herein, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. Treatment also includes prolonging survival as compared to expected survival if not receiving treatment or if receiving a different treatment. A treatment can include administration of immunotherapy and/or chemotherapy. A biomarker refers generally to a molecule, including without limitation a gene or product thereof, nucleic acids (e.g., DNA, RNA), protein/peptide/polypeptide, carbohydrate structure, lipid, glycolipid, characteristics of which can be detected in a tissue or cell to provide information that is predictive, diagnostic, prognostic and/or theranostic for sensitivity or resistance to candidate treatment.

Biological Samples

A sample as used herein includes any relevant biological sample that can be used for molecular profiling, e.g., sections of tissues such as biopsy or tissue removed during surgical or other procedures, bodily fluids, autopsy samples, and frozen sections taken for histological purposes. Such samples include blood and blood fractions or products (e.g., serum, buffy coat, plasma, platelets, red blood cells, and the like), sputum, malignant effusion, cheek cells tissue, cultured cells (e.g., primary cultures, explants, and transformed cells), stool, urine, other biological or bodily fluids (e.g., prostatic fluid, gastric fluid, intestinal fluid, renal fluid, lung fluid, cerebrospinal fluid, and the like), etc. The sample can comprise biological material that is a fresh frozen & formalin fixed paraffin embedded (FFPE) block, formalin-fixed paraffin embedded, or is within an RNA preservative+ formalin fixative. More than one sample of more than one type can be used for each patient. In a preferred embodiment, the sample comprises a fixed tumor sample.

The sample used in the systems and methods provided herein can be a formalin fixed paraffin embedded (FFPE) sample. The FFPE sample can be one or more of fixed tissue, unstained slides, bone marrow core or clot, core needle biopsy, malignant fluids and fine needle aspirate (FNA). In an embodiment, the fixed tissue comprises a tumor containing formalin fixed paraffin embedded (FFPE) block from a surgery or biopsy. In another embodiment, the unstained slides comprise unstained, charged, unbaked slides from a paraffin block. In another embodiment, bone marrow core or clot comprises a decalcified core. A formalin fixed core and/or clot can be paraffin-embedded. In still another embodiment, the core needle biopsy comprises 1, 2, 3, 4, 5, 6, 7, 8, 9, 10 or more, e.g., 3-4, paraffin embedded biopsy samples. An 18 gauge needle biopsy can be used. The malignant fluid can comprise a sufficient volume of fresh pleural/ascitic fluid to produce a 5×5×2 mm cell pellet. The fluid can be formalin fixed in a paraffin block. In an embodiment, the core needle biopsy comprises 1, 2, 3, 4, 5, 6, 7, 8, 9, 10 or more, e.g., 4-6, paraffin embedded aspirates.

A sample may be processed according to techniques understood by those in the art. A sample can be without limitation fresh, frozen or fixed cells or tissue. In some embodiments, a sample comprises formalin-fixed paraffin-embedded (FFPE) tissue, fresh tissue or fresh frozen (FF) tissue. A sample can comprise cultured cells, including primary or immortalized cell lines derived from a subject sample. A sample can also refer to an extract from a sample from a subject. For example, a sample can comprise DNA, RNA or protein extracted from a tissue or a bodily fluid. Many techniques and commercial kits are available for such purposes. The fresh sample from the individual can be treated with an agent to preserve RNA prior to further processing, e.g., cell lysis and extraction. Samples can include frozen samples collected for other purposes. Samples can be associated with relevant information such as age, gender, and clinical symptoms present in the subject; source of the sample; and methods of collection and storage of the sample. A sample is typically obtained from a subject.

A biopsy comprises the process of removing a tissue sample for diagnostic or prognostic evaluation, and to the tissue specimen itself. Any biopsy technique known in the art can be applied to the molecular profiling methods of the present disclosure. The biopsy technique applied can depend on the tissue type to be evaluated (e.g., colon, prostate, kidney, bladder, lymph node, liver, bone marrow, blood cell, lung, breast, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. Molecular profiling can use a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

Unless otherwise noted, a “sample” as referred to herein for molecular profiling of a patient may comprise more than one physical specimen. As one non-limiting example, a “sample” may comprise multiple sections from a tumor, e.g., multiple sections of an FFPE block or multiple core-needle biopsy sections. As another non-limiting example, a “sample” may comprise multiple biopsy specimens, e.g., one or more surgical biopsy specimen, one or more core-needle biopsy specimen, one or more fine-needle aspiration biopsy specimen, or any useful combination thereof. As still another non-limiting example, a molecular profile may be generated for a subject using a “sample” comprising a solid tumor specimen and a bodily fluid specimen. In some embodiments, a sample is a unitary sample, i.e., a single physical specimen.

Standard molecular biology techniques known in the art and not specifically described are generally followed as in Sambrook et al., Molecular Cloning: A Laboratory Manual, Cold Spring Harbor Laboratory Press, New York (1989), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) can be carried out generally as in PCR Protocols: A Guide to Methods and Applications, Academic Press, San Diego, Calif. (1990).

Circulating Biomarkers

Circulating biomarkers include biomarkers that are detectable in body fluids, such as blood, plasma, serum. Examples of circulating cancer biomarkers include cardiac troponin T (cTnT), prostate specific antigen (PSA) for prostate cancer and CA125 for ovarian cancer. Circulating biomarkers according to the present disclosure include any appropriate biomarker that can be detected in bodily fluid, including without limitation protein, nucleic acids, e.g., DNA, mRNA and microRNA, lipids, carbohydrates and metabolites. Circulating biomarkers can include biomarkers that are not associated with cells, such as biomarkers that are membrane associated, embedded in membrane fragments, part of a biological complex, or free in solution. For example, circulating biomarkers can be cell-free nucleic acids. In one embodiment, circulating biomarkers are biomarkers that are associated with one or more vesicles present in the biological fluid of a subject.

Circulating biomarkers have been identified for use in characterization of various phenotypes, such as detection of a cancer. See, e.g., Ahmed N, et al., Proteomic-based identification of haptoglobin-1 precursor as a novel circulating biomarker of ovarian cancer. Br. J. Cancer 2004; Mathelin et al., Circulating proteinic biomarkers and breast cancer, Gynecol Obstet Fertil. 2006 July-August; 34 (7-8): 638-46. Epub 2006 Jul. 28; Ye et al., Recent technical strategies to identify diagnostic biomarkers for ovarian cancer. Expert Rev Proteomics. 2007 February; 4(1):121-31; Carney, Circulating oncoproteins HER2/neu, EGFR and CAIX (MN) as novel cancer biomarkers. Expert Rev Mol Diagn. 2007 May; 7(3):309-19; Gagnon, Discovery and application of protein biomarkers for ovarian cancer, Curr Opin Obstet Gynecol. 2008 February; 20(1):9-13; Pasterkamp et al., Immune regulatory cells: circulating biomarker factories in cardiovascular disease. Clin Sci (Lond). 2008 August; 115(4):129-31; Fabbri, miRNAs as molecular biomarkers of cancer, Exp Rev Mol Diag, May 2010, Vol. 10, No. 4, Pages 435-444; PCT Patent Publication WO/2007/088537; U.S. Pat. Nos. 7,745,150 and 7,655,479; U.S. Patent Publications 20110008808, 20100330683, 20100248290, 20100222230, 20100203566, 20100173788, 20090291932, 20090239246, 20090226937, 20090111121, 20090004687, 20080261258, 20080213907, 20060003465, 20050124071, and 20040096915, each of which publication is incorporated herein by reference in its entirety. In an embodiment, molecular profiling as described herein comprises analysis of circulating biomarkers.

Gene Expression Profiling

The methods and systems as described herein comprise expression profiling, which includes assessing differential expression of one or more target genes disclosed herein. Differential expression can include overexpression and/or underexpression of a biological product, e.g., a gene, mRNA or protein, compared to a control (or a reference). The control can include similar cells to the sample but without the disease (e.g., expression profiles obtained from samples from healthy individuals). A control can be a previously determined level that is indicative of a drug target efficacy associated with the particular disease and the particular drug target. The control can be derived from the same patient, e.g., a normal adjacent portion of the same organ as the diseased cells, the control can be derived from healthy tissues from other patients, or previously determined thresholds that are indicative of a disease responding or not-responding to a particular drug target. The control can also be a control found in the same sample, e.g. a housekeeping gene or a product thereof (e.g., mRNA or protein). For example, a control nucleic acid can be one which is known not to differ depending on the cancerous or non-cancerous state of the cell. The expression level of a control nucleic acid can be used to normalize signal levels in the test and reference populations. Illustrative control genes include, but are not limited to, e.g., β-actin, glyceraldehyde 3-phosphate dehydrogenase and ribosomal protein P1. Multiple controls or types of controls can be used. The source of differential expression can vary. For example, a gene copy number may be increased in a cell, thereby resulting in increased expression of the gene. Alternately, transcription of the gene may be modified, e.g., by chromatin remodeling, differential methylation, differential expression or activity of transcription factors, etc. Translation may also be modified, e.g., by differential expression of factors that degrade mRNA, translate mRNA, or silence translation, e.g., microRNAs or siRNAs. In some embodiments, differential expression comprises differential activity. For example, a protein may carry a mutation that increases the activity of the protein, such as constitutive activation, thereby contributing to a diseased state. Molecular profiling that reveals changes in activity can be used to guide treatment selection.

Methods of gene expression profiling include methods based on hybridization analysis of polynucleotides, and methods based on sequencing of polynucleotides. Commonly used methods known in the art for the quantification of mRNA expression in a sample include northern blotting and in situ hybridization (Parker & Barnes (1999) Methods in Molecular Biology 106:247-283); RNAse protection assays (Hod (1992) Biotechniques 13:852-854); and reverse transcription polymerase chain reaction (RT-PCR) (Weis et al. (1992) Trends in Genetics 8:263-264). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), gene expression analysis by massively parallel signature sequencing (MPSS) and/or next generation sequencing.

DNA Copy Number Profiling

Any method capable of determining a DNA copy number profile of a particular sample can be used for molecular profiling according to the methods described herein as long as the resolution is sufficient to identify a copy number variation in the biomarkers as described herein. The skilled artisan is aware of and capable of using a number of different platforms for assessing whole genome copy number changes at a resolution sufficient to identify the copy number of the one or more biomarkers of the methods described herein. Some of the platforms and techniques are described in the embodiments below. In some embodiments, hybridization technologies, PCR techniques, next generation sequencing or ISH techniques can be used for determining copy number/gene amplification.

In some embodiments, the copy number profile analysis involves amplification of whole genome DNA by a whole genome amplification method. The whole genome amplification method can use a strand displacing polymerase and random primers.

In some aspects of these embodiments, the copy number profile analysis involves hybridization of whole genome amplified DNA with a high density array. In a more specific aspect, the high density array has 5,000 or more different probes. In another specific aspect, the high density array has 5,000, 10,000, 20,000, 50,000, 100,000, 200,000, 300,000, 400,000, 500,000, 600,000, 700,000, 800,000, 900,000, or 1,000,000 or more different probes. In another specific aspect, each of the different probes on the array is an oligonucleotide having from about 15 to 200 bases in length. In another specific aspect, each of the different probes on the array is an oligonucleotide having from about 15 to 200, 15 to 150, 15 to 100, 15 to 75, 15 to 60, or 20 to 55 bases in length.

In some embodiments, a microarray is employed to aid in determining the copy number profile for a sample, e.g., cells from a tumor. Microarrays typically comprise a plurality of oligomers (e.g., DNA or RNA polynucleotides or oligonucleotides, or other polymers), synthesized or deposited on a substrate (e.g., glass support) in an array pattern. The support-bound oligomers are “probes”, which function to hybridize or bind with a sample material (e.g., nucleic acids prepared or obtained from the tumor samples), in hybridization experiments. The reverse situation can also be applied: the sample can be bound to the microarray substrate and the oligomer probes are in solution for the hybridization. In use, the array surface is contacted with one or more targets under conditions that promote specific, high-affinity binding of the target to one or more of the probes. In some configurations, the sample nucleic acid is labeled with a detectable label, such as a fluorescent tag, so that the hybridized sample and probes are detectable with scanning equipment. DNA array technology offers the potential of using a multitude (e.g., hundreds of thousands) of different oligonucleotides to analyze DNA copy number profiles. In some embodiments, the substrates used for arrays are surface-derivatized glass or silica, or polymer membrane surfaces (see e.g., in Z. Guo, et al., Nucleic Acids Res, 22, 5456-65 (1994); U. Maskos, E. M. Southern, Nucleic Acids Res, 20, 1679-84 (1992), and E. M. Southern, et al., Nucleic Acids Res, 22, 1368-73 (1994), each incorporated by reference herein). Modification of surfaces of array substrates can be accomplished by many techniques. For example, siliceous or metal oxide surfaces can be derivatized with bifunctional silanes, i.e., silanes having a first functional group enabling covalent binding to the surface (e.g., Si-halogen or Si-alkoxy group, as in —SiCl₃ or —Si(OCH₃)₃, respectively) and a second functional group that can impart the desired chemical and/or physical modifications to the surface to covalently or non-covalently attach ligands and/or the polymers or monomers for the biological probe array. Silylated derivatizations and other surface derivatizations that are known in the art (see for example U.S. Pat. No. 5,624,711 to Sundberg, U.S. Pat. No. 5,266,222 to Willis, and U.S. Pat. No. 5,137,765 to Farnsworth, each incorporated by reference herein). Other processes for preparing arrays are described in U.S. Pat. No. 6,649,348, to Bass et. al., assigned to Agilent Corp., which disclose DNA arrays created by in situ synthesis methods.

Polymer array synthesis is also described extensively in the literature including in the following: WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, 5,412,087, 6,147,205, 6,262,216, 6,310,189, 5,889,165, and 5,959,098 in PCT Applications Nos. PCT/US99/00730 (International Publication No. WO 99/36760) and PCT/US01/04285 (International Publication No. WO 01/58593), which are all incorporated herein by reference in their entirety for all purposes.

Nucleic acid arrays that are useful in the present disclosure include, but are not limited to, those that are commercially available from Affymetrix (Santa Clara, Calif) under the brand name GeneChip™. Example arrays are shown on the website at affymetrix.com. Another microarray supplier is Illumina, Inc., of San Diego, Calif. with example arrays shown on their website at illumina.com.

In some embodiments, the inventive methods provide for sample preparation. Depending on the microarray and experiment to be performed, sample nucleic acid can be prepared in a number of ways by methods known to the skilled artisan. In some aspects as described herein, prior to or concurrent with genotyping (analysis of copy number profiles), the sample may be amplified any number of mechanisms. The most common amplification procedure used involves PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. In some embodiments, the sample may be amplified on the array (e.g., U.S. Pat. No. 6,300,070 which is incorporated herein by reference).

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. No. 10/013,598.

Methods for conducting polynucleotide hybridization assays are well developed in the art. Hybridization assay procedures and conditions used in the methods as described herein will vary depending on the application and are selected in accordance with the general binding methods known including those referred to in: Maniatis et al. Molecular Cloning: A Laboratory Manual (2.sup.nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The methods as described herein may also involve signal detection of hybridization between ligands in after (and/or during) hybridization. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

Sequence Analysis

Molecular profiling according to the present disclosure comprises methods for genotyping one or more biomarkers by determining whether an individual has one or more nucleotide variants (or amino acid variants) in one or more of the genes or gene products. Genotyping one or more genes according to the methods as described herein in some embodiments, can provide more evidence for selecting a treatment.

The biomarkers as described herein can be analyzed by any method useful for determining alterations in nucleic acids or the proteins they encode. According to one embodiment, the ordinary skilled artisan can analyze the one or more genes for mutations including deletion mutants, insertion mutants, frame shift mutants, nonsense mutants, missense mutant, and splice mutants.

Nucleic acid used for analysis of the one or more genes can be isolated from cells in the sample according to standard methodologies (Sambrook et al., 1989). The nucleic acid, for example, may be genomic DNA or fractionated or whole cell RNA, or miRNA acquired from exosomes or cell surfaces. Where RNA is used, it may be desired to convert the RNA to a complementary DNA. In one embodiment, the RNA is whole cell RNA; in another, it is poly-A RNA; in another, it is exosomal RNA. Normally, the nucleic acid is amplified. Depending on the format of the assay for analyzing the one or more genes, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects are known to occur in the biomarkers as described herein. Alterations include without limitation deletions, insertions, point mutations, and duplications. Point mutations can be silent or can result in stop codons, frame shift mutations or amino acid substitutions. Mutations in and outside the coding region of the one or more genes may occur and can be analyzed according to the methods as described herein. The target site of a nucleic acid of interest can include the region wherein the sequence varies. Examples include, but are not limited to, polymorphisms which exist in different forms such as single nucleotide variations, nucleotide repeats, multibase deletion (more than one nucleotide deleted from the consensus sequence), multibase insertion (more than one nucleotide inserted from the consensus sequence), microsatellite repeats (small numbers of nucleotide repeats with a typical 5-1000 repeat units), di-nucleotide repeats, tri-nucleotide repeats, sequence rearrangements (including translocation and duplication), chimeric sequence (two sequences from different gene origins are fused together), and the like. Among sequence polymorphisms, the most frequent polymorphisms in the human genome are single-base variations, also called single-nucleotide polymorphisms (SNPs). SNPs are abundant, stable and widely distributed across the genome.

Molecular profiling includes methods for haplotyping one or more genes. The haplotype is a set of genetic determinants located on a single chromosome and it typically contains a particular combination of alleles (all the alternative sequences of a gene) in a region of a chromosome. In other words, the haplotype is phased sequence information on individual chromosomes. Very often, phased SNPs on a chromosome define a haplotype. A combination of haplotypes on chromosomes can determine a genetic profile of a cell. It is the haplotype that determines a linkage between a specific genetic marker and a disease mutation. Haplotyping can be done by any methods known in the art. Common methods of scoring SNPs include hybridization microarray or direct gel sequencing, reviewed in Landgren et al., Genome Research, 8:769-776, 1998. For example, only one copy of one or more genes can be isolated from an individual and the nucleotide at each of the variant positions is determined. Alternatively, an allele specific PCR or a similar method can be used to amplify only one copy of the one or more genes in an individual, and SNPs at the variant positions of the present disclosure are determined. The Clark method known in the art can also be employed for haplotyping. A high throughput molecular haplotyping method is also disclosed in Tost et al., Nucleic Acids Res., 30(19): e96 (2002), which is incorporated herein by reference.

Thus, additional variant(s) that are in linkage disequilibrium with the variants and/or haplotypes of the present disclosure can be identified by a haplotyping method known in the art, as will be apparent to a skilled artisan in the field of genetics and haplotyping. The additional variants that are in linkage disequilibrium with a variant or haplotype of the present disclosure can also be useful in the various applications as described below.

For purposes of genotyping and haplotyping, both genomic DNA and mRNA/cDNA can be used, and both can be herein referred to generically as “gene.”

Numerous techniques for detecting nucleotide variants are known in the art and can all be used for the method of this disclosure. The techniques can be protein-based or nucleic acid-based. In either case, the techniques used must be sufficiently sensitive so as to accurately detect the small nucleotide or amino acid variations. Very often, a probe is used which is labeled with a detectable marker. Unless otherwise specified in a particular technique described below, any suitable marker known in the art can be used, including but not limited to, radioactive isotopes, fluorescent compounds, biotin which is detectable using streptavidin, enzymes (e.g., alkaline phosphatase), substrates of an enzyme, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977).

In a nucleic acid-based detection method, target DNA sample, i.e., a sample containing genomic DNA, cDNA, mRNA and/or miRNA, corresponding to the one or more genes must be obtained from the individual to be tested. Any tissue or cell sample containing the genomic DNA, miRNA, mRNA, and/or cDNA (or a portion thereof) corresponding to the one or more genes can be used. For this purpose, a tissue sample containing cell nucleus and thus genomic DNA can be obtained from the individual. Blood samples can also be useful except that only white blood cells and other lymphocytes have cell nucleus, while red blood cells are without a nucleus and contain only mRNA or miRNA. Nevertheless, miRNA and mRNA are also useful as either can be analyzed for the presence of nucleotide variants in its sequence or serve as template for cDNA synthesis. The tissue or cell samples can be analyzed directly without much processing. Alternatively, nucleic acids including the target sequence can be extracted, purified, and/or amplified before they are subject to the various detecting procedures discussed below. Other than tissue or cell samples, cDNAs or genomic DNAs from a cDNA or genomic DNA library constructed using a tissue or cell sample obtained from the individual to be tested are also useful.

To determine the presence or absence of a particular nucleotide variant, sequencing of the target genomic DNA or cDNA, particularly the region encompassing the nucleotide variant locus to be detected. Various sequencing techniques are generally known and widely used in the art including the Sanger method and Gilbert chemical method. The pyrosequencing method monitors DNA synthesis in real time using a luminometric detection system. Pyrosequencing has been shown to be effective in analyzing genetic polymorphisms such as single-nucleotide polymorphisms and can also be used in the present methods. See Nordstrom et al., Biotechnol. Appl. Biochem., 31(2):107-112 (2000); Ahmadian et al., Anal. Biochem., 280:103-110 (2000).

Nucleic acid variants can be detected by a suitable detection process. Non limiting examples of methods of detection, quantification, sequencing and the like are; mass detection of mass modified amplicons (e.g., matrix-assisted laser desorption ionization (MALDI) mass spectrometry and electrospray (ES) mass spectrometry), a primer extension method (e.g., iPLEX™; Sequenom, Inc.), microsequencing methods (e.g., a modification of primer extension methodology), ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), direct DNA sequencing, fragment analysis (FA), restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension (e.g., microarray sequence determination methods), Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Invader assay, hybridization methods (e.g., hybridization using at least one probe, hybridization using at least one fluorescently labeled probe, and the like), conventional dot blot analyses, single strand conformational polymorphism analysis (SSCP, e.g., U.S. Pat. Nos. 5,891,625 and 6,013,499; Orita et al., Proc. Natl. Acad. Sci. U.S.A. 86: 27776-2770 (1989)), denaturing gradient gel electrophoresis (DGGE), heteroduplex analysis, mismatch cleavage detection, and techniques described in Sheffield et al., Proc. Natl. Acad. Sci. USA 49: 699-706 (1991), White et al., Genomics 12: 301-306 (1992), Grompe et al., Proc. Natl. Acad. Sci. USA 86: 5855-5892 (1989), and Grompe, Nature Genetics 5: 111-117 (1993), cloning and sequencing, electrophoresis, the use of hybridization probes and quantitative real time polymerase chain reaction (QRT-PCR), digital PCR, nanopore sequencing, chips and combinations thereof. The detection and quantification of alleles or paralogs can be carried out using the “closed-tube” methods described in U.S. patent application Ser. No. 11/950,395, filed on Dec. 4, 2007. In some embodiments the amount of a nucleic acid species is determined by mass spectrometry, primer extension, sequencing (e.g., any suitable method, for example nanopore or pyrosequencing), Quantitative PCR (Q-PCR or QRT-PCR), digital PCR, combinations thereof, and the like.

The term “sequence analysis” as used herein refers to determining a nucleotide sequence, e.g., that of an amplification product. The entire sequence or a partial sequence of a polynucleotide, e.g., DNA or mRNA, can be determined, and the determined nucleotide sequence can be referred to as a “read” or “sequence read.” For example, linear amplification products may be analyzed directly without further amplification in some embodiments (e.g., by using single-molecule sequencing methodology). In certain embodiments, linear amplification products may be subject to further amplification and then analyzed (e.g., using sequencing by ligation or pyrosequencing methodology). Reads may be subject to different types of sequence analysis. Any suitable sequencing method can be used to detect, and determine the amount of, nucleotide sequence species, amplified nucleic acid species, or detectable products generated from the foregoing. Examples of certain sequencing methods are described hereafter.

A sequence analysis apparatus or sequence analysis component(s) includes an apparatus, and one or more components used in conjunction with such apparatus, that can be used by a person of ordinary skill to determine a nucleotide sequence resulting from processes described herein (e.g., linear and/or exponential amplification products). Examples of sequencing platforms include, without limitation, the 454 platform (Roche) (Margulies, M. et al. 2005 Nature 437, 376-380), Illumina Genomic Analyzer (or Solexa platform) or SOLID System (Applied Biosystems; see PCT patent application publications WO 06/084132 entitled “Reagents, Methods, and Libraries For Bead-Based Sequencing” and WO07/121,489 entitled “Reagents, Methods, and Libraries for Gel-Free Bead-Based Sequencing”), the Helicos True Single Molecule DNA sequencing technology (Harris T D et al. 2008 Science, 320, 106-109), the single molecule, real-time (SMRT™) technology of Pacific Biosciences, and nanopore sequencing (Soni G V and Meller A. 2007 Clin Chem 53: 1996-2001), Ion semiconductor sequencing (Ion Torrent Systems, Inc, San Francisco, CA), or DNA nanoball sequencing (Complete Genomics, Mountain View, CA), VisiGen Biotechnologies approach (Invitrogen) and polony sequencing. Such platforms allow sequencing of many nucleic acid molecules isolated from a specimen at high orders of multiplexing in a parallel manner (Dear Brief Funct Genomic Proteomic 2003; 1: 397-416; Haimovich, Methods, challenges, and promise of next-generation sequencing in cancer biology. Yale J Biol Med. 2011 December; 84(4):439-46). These non-Sanger-based sequencing technologies are sometimes referred to as NextGen sequencing, NGS, next-generation sequencing, next generation sequencing, and variations thereof. Typically they allow much higher throughput than the traditional Sanger approach. See Schuster, Next-generation sequencing transforms today's biology, Nature Methods 5:16-18 (2008); Metzker, Sequencing technologies—the next generation. Nat Rev Genet. 2010 January; 11(1):31-46; Levy and Myers, Advancements in Next-Generation Sequencing. Annu Rev Genomics Hum Genet. 2016 Aug. 31; 17:95-115. These platforms can allow sequencing of clonally expanded or non-amplified single molecules of nucleic acid fragments. Certain platforms involve, for example, sequencing by ligation of dye-modified probes (including cyclic ligation and cleavage), pyrosequencing, and single-molecule sequencing. Nucleotide sequence species, amplification nucleic acid species and detectable products generated there from can be analyzed by such sequence analysis platforms. Next-generation sequencing can be used in the methods as described herein, e.g., to determine mutations, copy number, or expression levels, as appropriate. The methods can be used to perform whole genome sequencing or sequencing of specific sequences of interest, such as a gene of interest or a fragment thereof.

Sequencing by ligation is a nucleic acid sequencing method that relies on the sensitivity of DNA ligase to base-pairing mismatch. DNA ligase joins together ends of DNA that are correctly base paired. Combining the ability of DNA ligase to join together only correctly base paired DNA ends, with mixed pools of fluorescently labeled oligonucleotides or primers, enables sequence determination by fluorescence detection. Longer sequence reads may be obtained by including primers containing cleavable linkages that can be cleaved after label identification. Cleavage at the linker removes the label and regenerates the 5′ phosphate on the end of the ligated primer, preparing the primer for another round of ligation. In some embodiments primers may be labeled with more than one fluorescent label, e.g., at least 1, 2, 3, 4, or 5 fluorescent labels.

Sequencing by ligation generally involves the following steps. Clonal bead populations can be prepared in emulsion microreactors containing target nucleic acid template sequences, amplification reaction components, beads and primers. After amplification, templates are denatured and bead enrichment is performed to separate beads with extended templates from undesired beads (e.g., beads with no extended templates). The template on the selected beads undergoes a 3′ modification to allow covalent bonding to the slide, and modified beads can be deposited onto a glass slide. Deposition chambers offer the ability to segment a slide into one, four or eight chambers during the bead loading process. For sequence analysis, primers hybridize to the adapter sequence. A set of four color dye-labeled probes competes for ligation to the sequencing primer. Specificity of probe ligation is achieved by interrogating every 4th and 5th base during the ligation series. Five to seven rounds of ligation, detection and cleavage record the color at every 5th position with the number of rounds determined by the type of library used. Following each round of ligation, a new complimentary primer offset by one base in the 5′ direction is laid down for another series of ligations. Primer reset and ligation rounds (5-7 ligation cycles per round) are repeated sequentially five times to generate 25-35 base pairs of sequence for a single tag. With mate-paired sequencing, this process is repeated for a second tag.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Target nucleic acids may be immobilized to a solid support, hybridized with a sequencing primer, incubated with DNA polymerase, ATP sulfurylase, luciferase, apyrase, adenosine 5′ phosphosulfate and luciferin. Nucleotide solutions are sequentially added and removed. Correct incorporation of a nucleotide releases a pyrophosphate, which interacts with ATP sulfurylase and produces ATP in the presence of adenosine 5′ phosphosulfate, fueling the luciferin reaction, which produces a chemiluminescent signal allowing sequence determination. The amount of light generated is proportional to the number of bases added. Accordingly, the sequence downstream of the sequencing primer can be determined. An illustrative system for pyrosequencing involves the following steps: ligating an adaptor nucleic acid to a nucleic acid under investigation and hybridizing the resulting nucleic acid to a bead; amplifying a nucleotide sequence in an emulsion; sorting beads using a picoliter multiwell solid support; and sequencing amplified nucleotide sequences by pyrosequencing methodology (e.g., Nakano et al., “Single-molecule PCR using water-in-oil emulsion;” Journal of Biotechnology 102: 117-124 (2003)).

Certain single-molecule sequencing embodiments are based on the principal of sequencing by synthesis, and use single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair” in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each for energy transfer to occur successfully.

An example of a system that can be used based on single-molecule sequencing generally involves hybridizing a primer to a target nucleic acid sequence to generate a complex; associating the complex with a solid phase; iteratively extending the primer by a nucleotide tagged with a fluorescent molecule; and capturing an image of fluorescence resonance energy transfer signals after each iteration (e.g., U.S. Pat. No. 7,169,314; Braslavsky et al., PNAS 100(7): 3960-3964 (2003)). Such a system can be used to directly sequence amplification products (linearly or exponentially amplified products) generated by processes described herein. In some embodiments the amplification products can be hybridized to a primer that contains sequences complementary to immobilized capture sequences present on a solid support, a bead or glass slide for example. Hybridization of the primer-amplification product complexes with the immobilized capture sequences, immobilizes amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer-amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

In some embodiments, nucleotide sequencing may be by solid phase single nucleotide sequencing methods and processes. Solid phase single nucleotide sequencing methods involve contacting target nucleic acid and solid support under conditions in which a single molecule of sample nucleic acid hybridizes to a single molecule of a solid support. Such conditions can include providing the solid support molecules and a single molecule of target nucleic acid in a “microreactor.” Such conditions also can include providing a mixture in which the target nucleic acid molecule can hybridize to solid phase nucleic acid on the solid support. Single nucleotide sequencing methods useful in the embodiments described herein are described in U.S. Provisional Patent Application Ser. No. 61/021,871 filed Jan. 17, 2008.

In certain embodiments, nanopore sequencing detection methods include (a) contacting a target nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected. In some embodiments, a detector disassociated from a base nucleic acid emits a detectable signal, and the detector hybridized to the base nucleic acid emits a different detectable signal or no detectable signal. In certain embodiments, nucleotides in a nucleic acid (e.g., linked probe molecule) are substituted with specific nucleotide sequences corresponding to specific nucleotides (“nucleotide representatives”), thereby giving rise to an expanded nucleic acid (e.g., U.S. Pat. No. 6,723,513), and the detectors hybridize to the nucleotide representatives in the expanded nucleic acid, which serves as a base nucleic acid. In such embodiments, nucleotide representatives may be arranged in a binary or higher order arrangement (e.g., Soni and Meller, Clinical Chemistry 53(11): 1996-2001 (2007)). In some embodiments, a nucleic acid is not expanded, does not give rise to an expanded nucleic acid, and directly serves a base nucleic acid (e.g., a linked probe molecule serves as a non-expanded base nucleic acid), and detectors are directly contacted with the base nucleic acid. For example, a first detector may hybridize to a first subsequence and a second detector may hybridize to a second subsequence, where the first detector and second detector each have detectable labels that can be distinguished from one another, and where the signals from the first detector and second detector can be distinguished from one another when the detectors are disassociated from the base nucleic acid. In certain embodiments, detectors include a region that hybridizes to the base nucleic acid (e.g., two regions), which can be about 3 to about 100 nucleotides in length (e.g., about 4, 5, 6, 7, 8, 9, 10, 11, 12, 13, 14, 15, 16, 17, 18, 19, 20, 25, 30, 35, 40, 50, 55, 60, 65, 70, 75, 80, 85, 90, or 95 nucleotides in length). A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

In certain sequence analysis embodiments, reads may be used to construct a larger nucleotide sequence, which can be facilitated by identifying overlapping sequences in different reads and by using identification sequences in the reads. Such sequence analysis methods and software for constructing larger sequences from reads are known to the person of ordinary skill (e.g., Venter et al., Science 291: 1304-1351 (2001)). Specific reads, partial nucleotide sequence constructs, and full nucleotide sequence constructs may be compared between nucleotide sequences within a sample nucleic acid (i.e., internal comparison) or may be compared with a reference sequence (i.e., reference comparison) in certain sequence analysis embodiments. Internal comparisons can be performed in situations where a sample nucleic acid is prepared from multiple samples or from a single sample source that contains sequence variations. Reference comparisons sometimes are performed when a reference nucleotide sequence is known and an objective is to determine whether a sample nucleic acid contains a nucleotide sequence that is substantially similar or the same, or different, than a reference nucleotide sequence. Sequence analysis can be facilitated by the use of sequence analysis apparatus and components described above.

Primer extension polymorphism detection methods, also referred to herein as “microsequencing” methods, typically are carried out by hybridizing a complementary oligonucleotide to a nucleic acid carrying the polymorphic site. In these methods, the oligonucleotide typically hybridizes adjacent to the polymorphic site. The term “adjacent” as used in reference to “microsequencing” methods, refers to the 3′ end of the extension oligonucleotide being sometimes 1 nucleotide from the 5′ end of the polymorphic site, often 2 or 3, and at times 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, often 1, 2, or 3 nucleotides, and the number and/or type of nucleotides that are added to the extension oligonucleotide determine which polymorphic variant or variants are present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. The extension products can be detected in any manner, such as by fluorescence methods (see, e.g., Chen & Kwok, Nucleic Acids Research 25: 347-353 (1997) and Chen et al., Proc. Natl. Acad. Sci. USA 94/20: 10756-10761 (1997)) or by mass spectrometric methods (e.g., MALDI-TOF mass spectrometry) and other methods described herein. Oligonucleotide extension methods using mass spectrometry are described, for example, in U.S. Pat. Nos. 5,547,835; 5,605,798; 5,691,141; 5,849,542; 5,869,242; 5,928,906; 6,043,031; 6,194,144; and 6,258,538.

Microsequencing detection methods often incorporate an amplification process that proceeds the extension step. The amplification process typically amplifies a region from a nucleic acid sample that comprises the polymorphic site. Amplification can be carried out using methods described above, or for example using a pair of oligonucleotide primers in a polymerase chain reaction (PCR), in which one oligonucleotide primer typically is complementary to a region 3′ of the polymorphism and the other typically is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GeneAmp™ Systems available from Applied Biosystems.

Other appropriate sequencing methods include multiplex polony sequencing (as described in Shendure et al., Accurate Multiplex Polony Sequencing of an Evolved Bacterial Genome, Sciencexpress, Aug. 4, 2005, pg 1 available at sciencexpress.org/4 Aug. 2005/Pagel/10.1126/science.1117389, incorporated herein by reference), which employs immobilized microbeads, and sequencing in microfabricated picoliter reactors (as described in Margulies et al., Genome Sequencing in Microfabricated High-Density Picolitre Reactors, Nature, August 2005, available at nature.com/nature (published online 31 Jul. 2005, doi:10.1038/nature03959, incorporated herein by reference).

Whole genome sequencing may also be used for discriminating alleles of RNA transcripts, in some embodiments. Examples of whole genome sequencing methods include, but are not limited to, nanopore-based sequencing methods, sequencing by synthesis and sequencing by ligation, as described above.

Nucleic acid variants can also be detected using standard electrophoretic techniques. Although the detection step can sometimes be preceded by an amplification step, amplification is not required in the embodiments described herein. Examples of methods for detection and quantification of a nucleic acid using electrophoretic techniques can be found in the art. A non-limiting example comprises running a sample (e.g., mixed nucleic acid sample isolated from maternal serum, or amplification nucleic acid species, for example) in an agarose or polyacrylamide gel. The gel may be labeled (e.g., stained) with ethidium bromide (see, Sambrook and Russell, Molecular Cloning: A Laboratory Manual 3d ed., 2001). The presence of a band of the same size as the standard control is an indication of the presence of a target nucleic acid sequence, the amount of which may then be compared to the control based on the intensity of the band, thus detecting and quantifying the target sequence of interest. In some embodiments, restriction enzymes capable of distinguishing between maternal and paternal alleles may be used to detect and quantify target nucleic acid species. In certain embodiments, oligonucleotide probes specific to a sequence of interest are used to detect the presence of the target sequence of interest. The oligonucleotides can also be used to indicate the amount of the target nucleic acid molecules in comparison to the standard control, based on the intensity of signal imparted by the probe.

Sequence-specific probe hybridization can be used to detect a particular nucleic acid in a mixture or mixed population comprising other species of nucleic acids. Under sufficiently stringent hybridization conditions, the probes hybridize specifically only to substantially complementary sequences. The stringency of the hybridization conditions can be relaxed to tolerate varying amounts of sequence mismatch. A number of hybridization formats are known in the art, which include but are not limited to, solution phase, solid phase, or mixed phase hybridization assays. The following articles provide an overview of the various hybridization assay formats: Singer et al., Biotechniques 4:230, 1986; Haase et al., Methods in Virology, pp. 189-226, 1984; Wilkinson, In situ Hybridization, Wilkinson ed., IRL Press, Oxford University Press, Oxford; and Hames and Higgins eds., Nucleic Acid Hybridization: A Practical Approach, IRL Press, 1987.

Hybridization complexes can be detected by techniques known in the art. Nucleic acid probes capable of specifically hybridizing to a target nucleic acid (e.g., mRNA or DNA) can be labeled by any suitable method, and the labeled probe used to detect the presence of hybridized nucleic acids. One commonly used method of detection is autoradiography, using probes labeled with ³H, ¹²⁵I, ³⁵S, ¹⁴C, ³²P ³³P, or the like. The choice of radioactive isotope depends on research preferences due to ease of synthesis, stability, and half-lives of the selected isotopes. Other labels include compounds (e.g., biotin and digoxigenin), which bind to antiligands or antibodies labeled with fluorophores, chemiluminescent agents, and enzymes. In some embodiments, probes can be conjugated directly with labels such as fluorophores, chemiluminescent agents or enzymes. The choice of label depends on sensitivity required, ease of conjugation with the probe, stability requirements, and available instrumentation.

In embodiments, fragment analysis (referred to herein as “FA”) methods are used for molecular profiling. Fragment analysis (FA) includes techniques such as restriction fragment length polymorphism (RFLP) and/or (amplified fragment length polymorphism). If a nucleotide variant in the target DNA corresponding to the one or more genes results in the elimination or creation of a restriction enzyme recognition site, then digestion of the target DNA with that particular restriction enzyme will generate an altered restriction fragment length pattern. Thus, a detected RFLP or AFLP will indicate the presence of a particular nucleotide variant.

Terminal restriction fragment length polymorphism (TRFLP) works by PCR amplification of DNA using primer pairs that have been labeled with fluorescent tags. The PCR products are digested using RFLP enzymes and the resulting patterns are visualized using a DNA sequencer. The results are analyzed either by counting and comparing bands or peaks in the TRFLP profile, or by comparing bands from one or more TRFLP runs in a database.

The sequence changes directly involved with an RFLP can also be analyzed more quickly by PCR. Amplification can be directed across the altered restriction site, and the products digested with the restriction enzyme. This method has been called Cleaved Amplified Polymorphic Sequence (CAPS). Alternatively, the amplified segment can be analyzed by Allele specific oligonucleotide (ASO) probes, a process that is sometimes assessed using a Dot blot.

A variation on AFLP is cDNA-AFLP, which can be used to quantify differences in gene expression levels.

Another useful approach is the single-stranded conformation polymorphism assay (SSCA), which is based on the altered mobility of a single-stranded target DNA spanning the nucleotide variant of interest. A single nucleotide change in the target sequence can result in different intramolecular base pairing pattern, and thus different secondary structure of the single-stranded DNA, which can be detected in a non-denaturing gel. See Orita et al., Proc. Natl. Acad. Sci. USA, 86:2776-2770 (1989). Denaturing gel-based techniques such as clamped denaturing gel electrophoresis (CDGE) and denaturing gradient gel electrophoresis (DGGE) detect differences in migration rates of mutant sequences as compared to wild-type sequences in denaturing gel. See Miller et al., Biotechniques, 5:1016-24 (1999); Sheffield et al., Am. J. Hum, Genet., 49:699-706 (1991); Wartell et al., Nucleic Acids Res., 18:2699-2705 (1990); and Sheffield et al., Proc. Natl. Acad. Sci. USA, 86:232-236 (1989). In addition, the double-strand conformation analysis (DSCA) can also be useful in the present methods. See Arguello et al., Nat. Genet., 18:192-194 (1998).

The presence or absence of a nucleotide variant at a particular locus in the one or more genes of an individual can also be detected using the amplification refractory mutation system (ARMS) technique. See e.g., European Patent No. 0,332,435; Newton et al., Nucleic Acids Res., 17:2503-2515 (1989); Fox et al., Br. J. Cancer, 77:1267-1274 (1998); Robertson et al., Eur. Respir. J., 12:477-482 (1998). In the ARMS method, a primer is synthesized matching the nucleotide sequence immediately 5′ upstream from the locus being tested except that the 3′-end nucleotide which corresponds to the nucleotide at the locus is a predetermined nucleotide. For example, the 3′-end nucleotide can be the same as that in the mutated locus. The primer can be of any suitable length so long as it hybridizes to the target DNA under stringent conditions only when its 3′-end nucleotide matches the nucleotide at the locus being tested. Preferably the primer has at least 12 nucleotides, more preferably from about 18 to 50 nucleotides. If the individual tested has a mutation at the locus and the nucleotide therein matches the 3′-end nucleotide of the primer, then the primer can be further extended upon hybridizing to the target DNA template, and the primer can initiate a PCR amplification reaction in conjunction with another suitable PCR primer. In contrast, if the nucleotide at the locus is of wild type, then primer extension cannot be achieved. Various forms of ARMS techniques developed in the past few years can be used. See e.g., Gibson et al., Clin. Chem. 43:1336-1341 (1997).

Similar to the ARMS technique is the mini sequencing or single nucleotide primer extension method, which is based on the incorporation of a single nucleotide. An oligonucleotide primer matching the nucleotide sequence immediately 5′ to the locus being tested is hybridized to the target DNA, mRNA or miRNA in the presence of labeled dideoxyribonucleotides. A labeled nucleotide is incorporated or linked to the primer only when the dideoxyribonucleotides matches the nucleotide at the variant locus being detected. Thus, the identity of the nucleotide at the variant locus can be revealed based on the detection label attached to the incorporated dideoxyribonucleotides. See Syvanen et al., Genomics, 8:684-692 (1990); Shumaker et al., Hum. Mutat., 7:346-354 (1996); Chen et al., Genome Res., 10:549-547 (2000).

Another set of techniques useful in the present methods is the so-called “oligonucleotide ligation assay” (OLA) in which differentiation between a wild-type locus and a mutation is based on the ability of two oligonucleotides to anneal adjacent to each other on the target DNA molecule allowing the two oligonucleotides joined together by a DNA ligase. See Landergren et al., Science, 241:1077-1080 (1988); Chen et al, Genome Res., 8:549-556 (1998); Iannone et al., Cytometry, 39:131-140 (2000). Thus, for example, to detect a single-nucleotide mutation at a particular locus in the one or more genes, two oligonucleotides can be synthesized, one having the sequence just 5′ upstream from the locus with its 3′ end nucleotide being identical to the nucleotide in the variant locus of the particular gene, the other having a nucleotide sequence matching the sequence immediately 3′ downstream from the locus in the gene. The oligonucleotides can be labeled for the purpose of detection. Upon hybridizing to the target gene under a stringent condition, the two oligonucleotides are subject to ligation in the presence of a suitable ligase. The ligation of the two oligonucleotides would indicate that the target DNA has a nucleotide variant at the locus being detected.

Detection of small genetic variations can also be accomplished by a variety of hybridization-based approaches. Allele-specific oligonucleotides are most useful. See Conner et al., Proc. Natl. Acad. Sci. USA, 80:278-282 (1983); Saiki et al, Proc. Natl. Acad. Sci. USA, 86:6230-6234 (1989). Oligonucleotide probes (allele-specific) hybridizing specifically to a gene allele having a particular gene variant at a particular locus but not to other alleles can be designed by methods known in the art. The probes can have a length of, e.g., from 10 to about 50 nucleotide bases. The target DNA and the oligonucleotide probe can be contacted with each other under conditions sufficiently stringent such that the nucleotide variant can be distinguished from the wild-type gene based on the presence or absence of hybridization. The probe can be labeled to provide detection signals. Alternatively, the allele-specific oligonucleotide probe can be used as a PCR amplification primer in an “allele-specific PCR” and the presence or absence of a PCR product of the expected length would indicate the presence or absence of a particular nucleotide variant.

Other useful hybridization-based techniques allow two single-stranded nucleic acids annealed together even in the presence of mismatch due to nucleotide substitution, insertion or deletion. The mismatch can then be detected using various techniques. For example, the annealed duplexes can be subject to electrophoresis. The mismatched duplexes can be detected based on their electrophoretic mobility that is different from the perfectly matched duplexes. See Cariello, Human Genetics, 42:726 (1988). Alternatively, in an RNase protection assay, a RNA probe can be prepared spanning the nucleotide variant site to be detected and having a detection marker. See Giunta et al., Diagn. Mol. Path., 5:265-270 (1996); Finkelstein et al., Genomics, 7:167-172 (1990); Kinszler et al., Science 251:1366-1370 (1991). The RNA probe can be hybridized to the target DNA or mRNA forming a heteroduplex that is then subject to the ribonuclease RNase A digestion. RNase A digests the RNA probe in the heteroduplex only at the site of mismatch. The digestion can be determined on a denaturing electrophoresis gel based on size variations. In addition, mismatches can also be detected by chemical cleavage methods known in the art. See e.g., Roberts et al., Nucleic Acids Res., 25:3377-3378 (1997).

In the mutS assay, a probe can be prepared matching the gene sequence surrounding the locus at which the presence or absence of a mutation is to be detected, except that a predetermined nucleotide is used at the variant locus. Upon annealing the probe to the target DNA to form a duplex, the E. coli mutS protein is contacted with the duplex. Since the mutS protein binds only to heteroduplex sequences containing a nucleotide mismatch, the binding of the mutS protein will be indicative of the presence of a mutation. See Modrich et al., Ann. Rev. Genet., 25:229-253 (1991).

A great variety of improvements and variations have been developed in the art on the basis of the above-described basic techniques which can be useful in detecting mutations or nucleotide variants in the present methods. For example, the “sunrise probes” or “molecular beacons” use the fluorescence resonance energy transfer (FRET) property and give rise to high sensitivity. See Wolf et al., Proc. Nat. Acad. Sci. USA, 85:8790-8794 (1988). Typically, a probe spanning the nucleotide locus to be detected are designed into a hairpin-shaped structure and labeled with a quenching fluorophore at one end and a reporter fluorophore at the other end. In its natural state, the fluorescence from the reporter fluorophore is quenched by the quenching fluorophore due to the proximity of one fluorophore to the other. Upon hybridization of the probe to the target DNA, the 5′ end is separated apart from the 3′-end and thus fluorescence signal is regenerated. See Nazarenko et al., Nucleic Acids Res., 25:2516-2521 (1997); Rychlik et al., Nucleic Acids Res., 17:8543-8551 (1989); Sharkey et al., Bio/Technology 12:506-509 (1994); Tyagi et al., Nat. Biotechnol., 14:303-308 (1996); Tyagi et al., Nat. Biotechnol., 16:49-53 (1998). The homo-tag assisted non-dimer system (HANDS) can be used in combination with the molecular beacon methods to suppress primer-dimer accumulation. See Brownie et al., Nucleic Acids Res., 25:3235-3241 (1997).

Dye-labeled oligonucleotide ligation assay is a FRET-based method, which combines the OLA assay and PCR. See Chen et al., Genome Res. 8:549-556 (1998). TaqMan is another FRET-based method for detecting nucleotide variants. A TaqMan probe can be oligonucleotides designed to have the nucleotide sequence of the gene spanning the variant locus of interest and to differentially hybridize with different alleles. The two ends of the probe are labeled with a quenching fluorophore and a reporter fluorophore, respectively. The TaqMan probe is incorporated into a PCR reaction for the amplification of a target gene region containing the locus of interest using Taq polymerase. As Taq polymerase exhibits 5′-3′ exonuclease activity but has no 3′-5′ exonuclease activity, if the TaqMan probe is annealed to the target DNA template, the 5′-end of the TaqMan probe will be degraded by Taq polymerase during the PCR reaction thus separating the reporting fluorophore from the quenching fluorophore and releasing fluorescence signals. See Holland et al., Proc. Natl. Acad. Sci. USA, 88:7276-7280 (1991); Kalinina et al., Nucleic Acids Res., 25:1999-2004 (1997); Whitcombe et al., Clin. Chem., 44:918-923 (1998).

In addition, the detection in the present methods can also employ a chemiluminescence-based technique. For example, an oligonucleotide probe can be designed to hybridize to either the wild-type or a variant gene locus but not both. The probe is labeled with a highly chemiluminescent acridinium ester. Hydrolysis of the acridinium ester destroys chemiluminescence. The hybridization of the probe to the target DNA prevents the hydrolysis of the acridinium ester. Therefore, the presence or absence of a particular mutation in the target DNA is determined by measuring chemiluminescence changes. See Nelson et al., Nucleic Acids Res., 24:4998-5003 (1996).

The detection of genetic variation in the gene in accordance with the present methods can also be based on the “base excision sequence scanning” (BESS) technique. The BESS method is a PCR-based mutation scanning method. BESS T-Scan and BESS G-Tracker are generated which are analogous to T and G ladders of dideoxy sequencing. Mutations are detected by comparing the sequence of normal and mutant DNA. See, e.g., Hawkins et al., Electrophoresis, 20:1171-1176 (1999).

Mass spectrometry can be used for molecular profiling according to the present methods. See Graber et al., Curr. Opin. Biotechnol., 9:14-18 (1998). For example, in the primer oligo base extension (PROBE™) method, a target nucleic acid is immobilized to a solid-phase support. A primer is annealed to the target immediately 5′ upstream from the locus to be analyzed. Primer extension is carried out in the presence of a selected mixture of deoxyribonucleotides and dideoxyribonucleotides. The resulting mixture of newly extended primers is then analyzed by MALDI-TOF. See e.g., Monforte et al., Nat. Med., 3:360-362 (1997).

In addition, the microchip or microarray technologies are also applicable to the detection method of the present methods. Essentially, in microchips, a large number of different oligonucleotide probes are immobilized in an array on a substrate or carrier, e.g., a silicon chip or glass slide. Target nucleic acid sequences to be analyzed can be contacted with the immobilized oligonucleotide probes on the microchip. See Lipshutz et al., Biotechniques, 19:442-447 (1995); Chee et al., Science, 274:610-614 (1996); Kozal et al., Nat. Med. 2:753-759 (1996); Hacia et al., Nat. Genet., 14:441-447 (1996); Saiki et al., Proc. Natl. Acad. Sci. USA, 86:6230-6234 (1989); Gingeras et al., Genome Res., 8:435-448 (1998). Alternatively, the multiple target nucleic acid sequences to be studied are fixed onto a substrate and an array of probes is contacted with the immobilized target sequences. See Drmanac et al., Nat. Biotechnol., 16:54-58 (1998). Numerous microchip technologies have been developed incorporating one or more of the above described techniques for detecting mutations. The microchip technologies combined with computerized analysis tools allow fast screening in a large scale. The adaptation of the microchip technologies to the present methods will be apparent to a person of skill in the art apprised of the present disclosure. See, e.g., U.S. Pat. No. 5,925,525 to Fodor et al; Wilgenbus et al., J. Mol. Med., 77:761-786 (1999); Graber et al., Curr. Opin. Biotechnol., 9:14-18 (1998); Hacia et al., Nat. Genet., 14:441-447 (1996); Shoemaker et al., Nat. Genet., 14:450-456 (1996); DeRisi et al., Nat. Genet., 14:457-460 (1996); Chee et al., Nat. Genet., 14:610-614 (1996); Lockhart et al., Nat. Genet., 14:675-680 (1996); Drobyshev et al., Gene, 188:45-52 (1997).

As is apparent from the above survey of the suitable detection techniques, it may or may not be necessary to amplify the target DNA, i.e., the gene, cDNA, mRNA, miRNA, or a portion thereof to increase the number of target DNA molecule, depending on the detection techniques used. For example, most PCR-based techniques combine the amplification of a portion of the target and the detection of the mutations. PCR amplification is well known in the art and is disclosed in U.S. Pat. Nos. 4,683,195 and 4,800,159, both which are incorporated herein by reference. For non-PCR-based detection techniques, if necessary, the amplification can be achieved by, e.g., in vivo plasmid multiplication, or by purifying the target DNA from a large amount of tissue or cell samples. See generally, Sambrook et al., Molecular Cloning: A Laboratory Manual, 2^{nd} ed., Cold Spring Harbor Laboratory, Cold Spring Harbor, N.Y., 1989.

However, even with scarce samples, many sensitive techniques have been developed in which small genetic variations such as single-nucleotide substitutions can be detected without having to amplify the target DNA in the sample. For example, techniques have been developed that amplify the signal as opposed to the target DNA by, e.g., employing branched DNA or dendrimers that can hybridize to the target DNA. The branched or dendrimer DNAs provide multiple hybridization sites for hybridization probes to attach thereto thus amplifying the detection signals. See Detmer et al., J. Clin. Microbiol., 34:901-907 (1996); Collins et al., Nucleic Acids Res., 25:2979-2984 (1997); Horn et al., Nucleic Acids Res., 25:4835-4841 (1997); Horn et al., Nucleic Acids Res., 25:4842-4849 (1997); Nilsen et al., J. Theor. Biol., 187:273-284 (1997).

The Invader™ assay is another technique for detecting single nucleotide variations that can be used for molecular profiling according to the methods. The Invader™ assay uses a novel linear signal amplification technology that improves upon the long turnaround times required of the typical PCR DNA sequenced-based analysis. See Cooksey et al., Antimicrobial Agents and Chemotherapy 44:1296-1301 (2000). This assay is based on cleavage of a unique secondary structure formed between two overlapping oligonucleotides that hybridize to the target sequence of interest to form a “flap.” Each “flap” then generates thousands of signals per hour. Thus, the results of this technique can be easily read, and the methods do not require exponential amplification of the DNA target. The Invader™ system uses two short DNA probes, which are hybridized to a DNA target. The structure formed by the hybridization event is recognized by a special cleavase enzyme that cuts one of the probes to release a short DNA “flap.” Each released “flap” then binds to a fluorescently-labeled probe to form another cleavage structure. When the cleavase enzyme cuts the labeled probe, the probe emits a detectable fluorescence signal. See e.g. Lyamichev et al., Nat. Biotechnol., 17:292-296 (1999).

The rolling circle method is another method that avoids exponential amplification. Lizardi et al., Nature Genetics, 19:225-232 (1998) (which is incorporated herein by reference). For example, Sniper™, a commercial embodiment of this method, is a sensitive, high-throughput SNP scoring system designed for the accurate fluorescent detection of specific variants. For each nucleotide variant, two linear, allele-specific probes are designed. The two allele-specific probes are identical with the exception of the 3′-base, which is varied to complement the variant site. In the first stage of the assay, target DNA is denatured and then hybridized with a pair of single, allele-specific, open-circle oligonucleotide probes. When the 3′-base exactly complements the target DNA, ligation of the probe will preferentially occur. Subsequent detection of the circularized oligonucleotide probes is by rolling circle amplification, whereupon the amplified probe products are detected by fluorescence. See Clark and Pickering, Life Science News 6, 2000, Amersham Pharmacia Biotech (2000).

A number of other techniques that avoid amplification all together include, e.g., surface-enhanced resonance Raman scattering (SERRS), fluorescence correlation spectroscopy, and single-molecule electrophoresis. In SERRS, a chromophore-nucleic acid conjugate is absorbed onto colloidal silver and is irradiated with laser light at a resonant frequency of the chromophore. See Graham et al., Anal. Chem., 69:4703-4707 (1997). The fluorescence correlation spectroscopy is based on the spatio-temporal correlations among fluctuating light signals and trapping single molecules in an electric field. See Eigen et al., Proc. Natl. Acad. Sci. USA, 91:5740-5747 (1994). In single-molecule electrophoresis, the electrophoretic velocity of a fluorescently tagged nucleic acid is determined by measuring the time required for the molecule to travel a predetermined distance between two laser beams. See Castro et al., Anal. Chem., 67:3181-3186 (1995).

In addition, the allele-specific oligonucleotides (ASO) can also be used in in situ hybridization using tissues or cells as samples. The oligonucleotide probes which can hybridize differentially with the wild-type gene sequence or the gene sequence harboring a mutation may be labeled with radioactive isotopes, fluorescence, or other detectable markers. In situ hybridization techniques are well known in the art and their adaptation to the present methods for detecting the presence or absence of a nucleotide variant in the one or more gene of a particular individual should be apparent to a skilled artisan apprised of this disclosure.

Accordingly, the presence or absence of one or more genes nucleotide variant or amino acid variant in an individual can be determined using any of the detection methods described above.

Typically, once the presence or absence of one or more gene nucleotide variants or amino acid variants is determined, physicians or genetic counselors or patients or other researchers may be informed of the result. Specifically the result can be cast in a transmittable form that can be communicated or transmitted to other researchers or physicians or genetic counselors or patients. Such a form can vary and can be tangible or intangible. The result with regard to the presence or absence of a nucleotide variant of the present methods in the individual tested can be embodied in descriptive statements, diagrams, photographs, charts, images or any other visual forms. For example, images of gel electrophoresis of PCR products can be used in explaining the results. Diagrams showing where a variant occurs in an individual's gene are also useful in indicating the testing results. The statements and visual forms can be recorded on a tangible media such as papers, computer readable media such as floppy disks, compact disks, etc., or on an intangible media, e.g., an electronic media in the form of email or website on internet or intranet. In addition, the result with regard to the presence or absence of a nucleotide variant or amino acid variant in the individual tested can also be recorded in a sound form and transmitted through any suitable media, e.g., analog or digital cable lines, fiber optic cables, etc., via telephone, facsimile, wireless mobile phone, internet phone and the like.

Thus, the information and data on a test result can be produced anywhere in the world and transmitted to a different location. For example, when a genotyping assay is conducted offshore, the information and data on a test result may be generated and cast in a transmittable form as described above. The test result in a transmittable form thus can be imported into the U.S. Accordingly, the present methods also encompasses a method for producing a transmittable form of information on the genotype of the two or more suspected cancer samples from an individual. The method comprises the steps of (1) determining the genotype of the DNA from the samples according to methods of the present methods; and (2) embodying the result of the determining step in a transmittable form. The transmittable form is the product of the production method.

In Situ Hybridization

In situ hybridization assays are well known and are generally described in Angerer et al., Methods Enzymol. 152:649-660 (1987). In an in situ hybridization assay, cells, e.g., from a biopsy, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled, e.g., with radioisotopes or fluorescent reporters, or enzymatically. FISH (fluorescence in situ hybridization) uses fluorescent probes that bind to only those parts of a sequence with which they show a high degree of sequence similarity. CISH (chromogenic in situ hybridization) uses conventional peroxidase or alkaline phosphatase reactions visualized under a standard bright-field microscope.

In situ hybridization can be used to detect specific gene sequences in tissue sections or cell preparations by hybridizing the complementary strand of a nucleotide probe to the sequence of interest. Fluorescent in situ hybridization (FISH) uses a fluorescent probe to increase the sensitivity of in situ hybridization.

FISH is a cytogenetic technique used to detect and localize specific polynucleotide sequences in cells. For example, FISH can be used to detect DNA sequences on chromosomes. FISH can also be used to detect and localize specific RNAs, e.g., mRNAs, within tissue samples. In FISH uses fluorescent probes that bind to specific nucleotide sequences to which they show a high degree of sequence similarity. Fluorescence microscopy can be used to find out whether and where the fluorescent probes are bound. In addition to detecting specific nucleotide sequences, e.g., translocations, fusion, breaks, duplications and other chromosomal abnormalities, FISH can help define the spatial-temporal patterns of specific gene copy number and/or gene expression within cells and tissues.

Various types of FISH probes can be used to detect chromosome translocations. Dual color, single fusion probes can be useful in detecting cells possessing a specific chromosomal translocation. The DNA probe hybridization targets are located on one side of each of the two genetic breakpoints. “Extra signal” probes can reduce the frequency of normal cells exhibiting an abnormal FISH pattern due to the random co-localization of probe signals in a normal nucleus. One large probe spans one breakpoint, while the other probe flanks the breakpoint on the other gene. Dual color, break apart probes are useful in cases where there may be multiple translocation partners associated with a known genetic breakpoint. This labeling scheme features two differently colored probes that hybridize to targets on opposite sides of a breakpoint in one gene. Dual color, dual fusion probes can reduce the number of normal nuclei exhibiting abnormal signal patterns. The probe offers advantages in detecting low levels of nuclei possessing a simple balanced translocation. Large probes span two breakpoints on different chromosomes. Such probes are available as Vysis probes from Abbott Laboratories, Abbott Park, IL.

CISH, or chromogenic in situ hybridization, is a process in which a labeled complementary DNA or RNA strand is used to localize a specific DNA or RNA sequence in a tissue specimen. CISH methodology can be used to evaluate gene amplification, gene deletion, chromosome translocation, and chromosome number. CISH can use conventional enzymatic detection methodology, e.g., horseradish peroxidase or alkaline phosphatase reactions, visualized under a standard bright-field microscope. In a common embodiment, a probe that recognizes the sequence of interest is contacted with a sample. An antibody or other binding agent that recognizes the probe, e.g., via a label carried by the probe, can be used to target an enzymatic detection system to the site of the probe. In some systems, the antibody can recognize the label of a FISH probe, thereby allowing a sample to be analyzed using both FISH and CISH detection. CISH can be used to evaluate nucleic acids in multiple settings, e.g., formalin-fixed, paraffin-embedded (FFPE) tissue, blood or bone marrow smear, metaphase chromosome spread, and/or fixed cells.

In an embodiment, CISH is performed following the methodology in the SPoT-Light® HER2 CISH Kit available from Life Technologies (Carlsbad, CA) or similar CISH products available from Life Technologies. The SPoT-Light® HER2 CISH Kit itself is FDA approved for in vitro diagnostics and can be used for molecular profiling of HER2. CISH can be used in similar applications as FISH. Thus, one of skill will appreciate that reference to molecular profiling using FISH herein can be performed using CISH, unless otherwise specified.

Silver-enhanced in situ hybridization (SISH) is similar to CISH, but with SISH the signal appears as a black coloration due to silver precipitation instead of the chromogen precipitates of CISH.

Modifications of the in situ hybridization techniques can be used for molecular profiling according to the systems and methods provided herein. Such modifications comprise simultaneous detection of multiple targets, e.g., Dual ISH, Dual color CISH, bright field double in situ hybridization (BDISH). See e.g., the FDA approved INFORM HER2 Dual ISH DNA Probe Cocktail kit from Ventana Medical Systems, Inc. (Tucson, AZ); DuoCISH™, a dual color CISH kit developed by Dako Denmark A/S (Denmark).

Comparative Genomic Hybridization (CGH) comprises a molecular cytogenetic method of screening tumor samples for genetic changes showing characteristic patterns for copy number changes at chromosomal and subchromosomal levels. Alterations in patterns can be classified as DNA gains and losses. CGH employs the kinetics of in situ hybridization to compare the copy numbers of different DNA or RNA sequences from a sample, or the copy numbers of different DNA or RNA sequences in one sample to the copy numbers of the substantially identical sequences in another sample. In many useful applications of CGH, the DNA or RNA is isolated from a subject cell or cell population. The comparisons can be qualitative or quantitative. Procedures are described that permit determination of the absolute copy numbers of DNA sequences throughout the genome of a cell or cell population if the absolute copy number is known or determined for one or several sequences. The different sequences are discriminated from each other by the different locations of their binding sites when hybridized to a reference genome, usually metaphase chromosomes but in certain cases interphase nuclei. The copy number information originates from comparisons of the intensities of the hybridization signals among the different locations on the reference genome. The methods, techniques and applications of CGH are known, such as described in U.S. Pat. No. 6,335,167, and in U.S. App. Ser. No. 60/804,818, the relevant parts of which are herein incorporated by reference.

In an embodiment, CGH used to compare nucleic acids between diseased and healthy tissues. The method comprises isolating DNA from disease tissues (e.g., tumors) and reference tissues (e.g., healthy tissue) and labeling each with a different “color” or fluor. The two samples are mixed and hybridized to normal metaphase chromosomes. In the case of array or matrix CGH, the hybridization mixing is done on a slide with thousands of DNA probes. A variety of detection system can be used that basically determine the color ratio along the chromosomes to determine DNA regions that might be gained or lost in the diseased samples as compared to the reference.

Molecular Profiling for Treatment Selection

A cancer in a subject can be characterized by obtaining a biological sample from a subject and analyzing one or more biomarkers from the sample. For example, characterizing a cancer for a subject or individual can include identifying appropriate treatments or treatment efficacy for specific diseases, conditions, disease stages and condition stages, predictions and likelihood analysis of disease progression, particularly disease recurrence, metastatic spread or disease relapse. The products and processes described herein allow assessment of a subject on an individual basis, which can provide benefits of more efficient and economical decisions in treatment.

In an aspect, characterizing a cancer includes predicting whether a subject is likely to benefit from a treatment for the cancer. Biomarkers can be analyzed in the subject and their states can be compared to states of the biomarker of previous subjects that were known to benefit or not from a treatment. If the biomarker states or profiles of multiple biomarkers in a subject more closely aligns with those of previous subjects that were known to benefit from the treatment, the subject can be characterized, or predicted, as a one who is likely to benefit from the treatment. Similarly, if the biomarker states or profiles of multiple biomarkers in the subject more closely aligns with those of previous subjects that did not benefit from the treatment, the subject can be characterized, or predicted as one who may not benefit from the treatment. The sample used for characterizing a cancer can be any useful sample, such as tumor cells or circulating biomarkers such as cell free nucleic acids.

The methods can further include administering the selected treatment to the subject. Various immunotherapies, e.g., checkpoint inhibitor therapies such as ipilimumab, nivolumab, pembrolizumab, atezolizumab, avelumab, cemiplimab, and durvalumab, are FDA approved and others are in clinical trials or developmental stages. In embodiments, immunotherapy and/or chemotherapy regimens are administered.

The present disclosure describes the use of systems and methods to predict cancer patients as responders or non-responders to immunotherapy and/or chemotherapy treatment. Benefit is a relative term and indicates that a treatment has a positive influence in treating a patient with cancer, but does not require complete remission. Similarly, lack of benefit can mean that the cancer will not improve or will progress on the treatment. A subject that receives a benefit may be referred to as a benefiter, responder, or the like. Likewise, a subject unlikely to receive a benefit or that does not benefit may be referred to herein as a non-benefiter, non-responder, or similar. The inventors found that loss on chromosome 9 can be detrimental to the efficacy of immunotherapy. See, e.g., Example 2. In an aspect, provided herein is a method of treating a cancer in a subject, the method comprising: (a) obtaining a biological sample comprising cells and/or cell free materials derived from the cancer in the subject; (b) performing an assay to assess a copy number of chromosome 9 or a portion thereof in the biological sample; and (c) administering a treatment for the cancer to the subject based on the assessment of step (b). In a related aspect, provided herein is a method of selecting a treatment for a subject who has a cancer, the method comprising: (a) obtaining a biological sample comprising cells and/or cell free material derived from the cancer in the subject; (b) performing an assay to assess a copy number of chromosome 9 or a portion thereof in the biological sample; and (c) selecting a treatment for the cancer to the subject based on the copy number of chromosome 9 or the portion thereof in (b). The disclosure also provides a method of generating a molecular profiling report comprising preparing a report summarizing results of performing such methods. The disclosure also provides a system comprising one or more computers and one or more storage media storing instructions that, when executed by the one or more computers, cause the one or more computers to perform operations in order to carry out the methods provided herein.

Cells are typically diploid with two copies of each gene. However, cancer may lead to various genomic alterations which can alter copy number. In some instances, copies of genes are amplified (gained), whereas in other instances copies of genes are lost. A reference for comparison to determine a copy number variation (CNV; or copy number alteration; CNA), such as gain or loss or no change, can be the diploid state. In some embodiments, the copy number loss is a complete loss (e.g., the loss of two copies of a genomic region in a diploid). In some embodiments, the copy number loss is a partial loss (e.g., the loss of one copy or a portion thereof of a genomic region in a diploid).

In some embodiments of the methods provided herein, assessing a copy number of chromosome 9 or a portion comprises determining a copy number of chromosome 9p or a portion thereof. The portion of chromosome 9 may be chromosome band 9p24 or a portion thereof. Band 9p24 is at the distal end of the short arm of chromosome 9. Thus 9p24 may be lost in terminal loss of 9p.

Copy number variations can detected using any number of assay technologies. For example, the assay may comprise sequencing, hybridization, or amplification of genomic DNA. In preferred embodiments, next-generation sequencing (NGS), including use of bait sets targeted to certain genes or regions of interest, whole-genome sequencing (WGS), whole-exome sequencing (WES), or any useful combination thereof. For example, a useful combination include WGS or WES with a bait set to boost coverage of certain genes or regions of interest. Copy number can also be queried using dye termination sequencing, pyrosequencing, in situ hybridization (ISH) and variants such as FISH or CISH, comparative genomic hybridization (CGH), high-resolution array comparative genomic hybridization (aCGH), microarray-based platforms, and PCR techniques. Copy number variations may be qualitative or quantitative. In non-limiting examples, visualization via ISH may reveal terminal loss of 9p, whereas copy number determined using NGS of a biological sample may be a numerical range, e.g., a normalized copy number determined around a reference such as copy number of 2 (diploid state).

The copy number of the portion of chromosome 9 may be determined by assessing various genes located in or around band 9p24. Thus, the portion of chromosome 9p24 comprises one or more genes located in chromosome band 9p24. In some embodiments, the one or more genes comprises PD-L1, JAK2, or PD-L1 and JAK2. In some embodiments, the one or more genes consists of PD-L1, JAK2, or PD-L1 and JAK2. See, e.g., Example 2 herein.

The one or more genes comprise any useful selection of genes found in chromosome 9, arm 9p or band 9p24. In some embodiments, the one or more genes are selected from those genes found in 9p24, including DDX11L5, WASHC1, MIR1302-9HG, MIR1302-9, FAM138C, PGM5P3-AS1, PGM5P3, LINC01388, FOXD4, CBWD1, LOC105375942, LOC105375943, DOCK8, DOCK8-AS1, LOC105375945, LOC112268042, KANK1, RPL12P25, FAM217AP1, LOC105375947, RNU6-1327P, EIF1P1, LOC107987042, LOC105375949, DMRT1, DMRT3, RNU6-1073P, DMRT2, H3P29, LINC01230, RPS27AP14, LOC102723803, RNA5SP279, LOC105375951, LOC105375953, LOC105375952, SMARCA2, RNU2-25P, LOC107987043, RN7SL592P, LOC105375955, LOC101930053, LOC105375956, LOC101930048, VLDLR-AS1, VLDLR, LOC105375957, KCNV2, PUM3, GPS2P1, ATP5PDP2, CARM1P1, LINC01231, LOC105375959, RFX3, RFX3-AS1, LOC105375962, GLIS3, GLIS3-AS1, LOC105375964, LOC107986989, RNU6-694P, SLC1A1, SPATA6L, RPS6P11, PLPP6, CDC37L1-DT, CDC37L1, AK3, ECM1P1, RPS5P6, RCL1, KLF4P1, MIR101-2, HNRNPA1P41, JAK2, INSL6, CSNK1G2P1, PDSS1P1, MTND6P5, MTND5P36, MTND1P11, MTND2P36, MTCO1P11, MTCO2P11, MTATP6P11, MTCO3P11, MTND3P14, MTND4LP6, MTND4P14, MTND5P14, TCF3P1, LOC107987044, IGHEP2, INSL4, RLN2, HMGN2P31, RLN1, PLGRKT, RNF152P1, CD274 (PD-L1), PDCD1LG2 (PD-L2), RIC1, ERMP1, AK4P4, KIAA2026, MLANA, MIR4665, RANBP6, GTF3AP1, IL33, LOC107987046, SELENOTP1, TPD52L3, UHRF2, GLDC, RN7SL25P, RPL23AP57, RN7SL123P, RNF2P1, RPL35AP20, LINC02851, KDM4C, PRELID3BP11, SNRPEP2, ACTG1P14, LOC105375969, LOC105375970, LOC102723994, RPL4P5, PPIAP33, DMAC1, LOC105375971, PTPRD, RPL18AP11, RNU7-185P, PTPRD-AS1, and any useful combination thereof.

Gene identifiers used herein are those commonly accepted in the scientific community at the time of filing and can be used to look up the genes at various well-known databases such as the HUGO Gene Nomenclature Committee (HNGC; genenames.org), NCBI's Gene database (ncbi.nlm.nih.gov/gene), GeneCards (genecards.org), Ensembl (ensembl.org), UniProt (uniprot.org), and others.

Various losses have been observed in cancer and other disease states, including truncation of a chromosomal arm or loss of the end of an arm. Genomic alterations can affect different regions of a chromosome. For example, gain or loss may occur within a gene, at the gene level, or within groups of neighboring genes. Gain or loss may be observed at the level of cytogenetic bands or even larger portions of chromosomal arms, including loss of a distal end of an arm. Thus, analysis of such proximate regions to a gene may provide similar or even identical information to the gene itself. Accordingly, the methods provided herein are not limited to determining copy number of the specified genes, but also expressly contemplate the analysis of proximate regions to the genes, wherein such proximate regions provide similar or the same level of information. As a non-limiting example, detection of loss at 9p23 or any portion therein, particularly distal loss, may provide similar information as loss of 9p24.

In some embodiments, assessing a copy number of chromosome 9 or portions thereof may comprise indirect measurement. For example, the presence or level of gene products of genes in 9p24, including, without limitation, RNA transcripts or proteins, may serve as proxies for loss of genomic DNA. In a non-limiting example, under-expressed or unexpressed gene products for PD-L1 and/or JAK2 may serve as proxies for direct assessment of genomic DNA. Thus, the assessment may comprise determining a presence, level, or state of a protein or nucleic acid for each biomarker. The nucleic acid can be deoxyribonucleic acid (DNA), ribonucleic acid (RNA), or a combination thereof. Any form of such nucleic acids that yields the desired information can be assessed, including without limitation coding RNA, non-coding RNA, mRNA, microRNA, lncRNA, snoRNA, or other forms. The presence, level or state of the genes and/or gene products can be measured with any useful technique. For example, protein can be assessed using immunohistochemistry (IHC), flow cytometry, an immunoassay, an antibody or functional fragment thereof, an aptamer, or any combination thereof. Additional useful techniques for assessing proteins are disclosed herein or known to those of skill in the art. As another example, the presence, level or state of nucleic acids can determined using polymerase chain reaction (PCR), in situ hybridization, amplification, hybridization, microarray, nucleic acid sequencing, dye termination sequencing, pyrosequencing, next generation sequencing (NGS; high-throughput sequencing), whole exome sequencing, whole transcriptome sequencing (WTS), or any combination thereof. Additional useful techniques for assessing nucleic acids are disclosed herein or known to those of skill in the art.

In addition to copy number, any useful state of the genes and gene products can be assessed. Non-limiting examples of the state of the nucleic acid include a sequence, mutation, polymorphism, deletion, insertion, substitution, translocation, fusion, break, duplication, amplification, repeat, copy number, copy number variation (CNV; copy number alteration; CNA), or any combination thereof. In various embodiments, high throughput sequencing techniques, e.g., next generation sequencing (NGS), including whole exome sequencing and/or whole transcriptome sequencing, can be used to assess some or all of these characteristics in a single assay. For example, a comprehensive molecular profile of a cancer as described in Example 1 can be performed, and the copy number of chromosome 9 or a desired portion thereof can be determined using the molecular profile. Alternate information gained from the molecular profiling can be used to help guide treatment of the cancer patient as desired. Various systems and methods for molecular profiling in order to select treatment options are described herein (see, e.g., Example 1) or described in any one of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; WO/2016/141169 (Int'l Appl. No. PCT/US2016/020657), published Sep. 9, 2016; and WO2018175501 (Int'l Appl. No. PCT/US2018/023438), published Sep. 27, 2018; Int'l Patent Appl. No. PCT/US2020/012815, filed Jan. 8, 2020; Int'l Patent Appl. No. PCT/US2021/018263, filed Feb. 16, 2021; Int'l Patent Appl. No. PCT/US2019/064078, filed Dec. 2, 2019; Int'l Patent Appl. No. PCT/US2020/035990, filed Jun. 3, 2020; Int'l Patent Appl. No. PCT/US2021/030351, filed Apr. 30, 2021; Int'l Patent Appl. No. PCT/US2021/049966, filed Sep. 10, 2021; each of which applications is incorporated by reference herein in its entirety. Such systems are methods for molecular profiling can be integrated with those provided herein.

The methods and systems provided herein may further comprise predicting whether the subject will benefit or not benefit from administration of an immunotherapy. See Example 2; Example 3. In some embodiments, loss of copy number of chromosome 9 or the portion thereof indicates lack of benefit of the immunotherapy. In some embodiments, the absence of loss of copy number of chromosome 9 or the portion thereof indicates potential response to the immunotherapy. The threshold for loss of copy number can be determined using a statistical model, such as a machine learning model. Such threshold can be more refined than simple comparison to diploid state, e.g., by providing a level of confidence. In some embodiments, the threshold is two copies, and any number of copies lower than two indicates a loss of copy number. Thus in some embodiments, the methods include detecting a number of copies below two, e.g., one (1) or zero (0) copy, in a sample from a subject, and identifying a subject who has 0 or 1 copy of chromosome 9 or a portion thereof, e.g., as described herein, as a subject who is not likely to respond to immunotherapy and should be administered a treatment other than immunotherapy (e.g., radiotherapy, chemotherapy, or surgical resection). Subjects who are identified as having a normal copy number (e.g., 2), or a gain of copy number (more than 2), can be identified as a subject who is likely to respond to immunotherapy and should be administered a treatment comprising immunotherapy (and optionally another therapy (e.g., radiotherapy, chemotherapy, or surgical resection).

In preferred embodiments, the immunotherapy comprises an immune checkpoint therapy. Immune checkpoints are pathways that inhibit or stimulate self-tolerance and assist with immune response. Well-known checkpoint inhibitor proteins include CTLA-4, PD-1, and PD-L1. Therapeutics that block checkpoint pathways can enhance the host immunologic activity against tumors. In some embodiments, the immune checkpoint therapy comprises at least one of anti-PD-1 therapy, anti-PD-L1 therapy, anti-CTLA-4 therapy, ipilimumab, nivolumab, pembrolizumab, atezolizumab, avelumab, durvalumab, cemiplimab, and any combination thereof. Many other such agents in development are contemplated within the scope of the systems and methods provided herein. See, e.g., Vaddepally R K, et al. Review of Indications of FDA-Approved Immune Checkpoint Inhibitors per NCCN Guidelines with the Level of Evidence. Cancers (Basel). 2020 Mar. 20; 12(3):738; Zam W, and Ali L. Immune checkpoint inhibitors in the treatment of cancer. Curr Clin Pharmacol. 2021 Mar. 24; Marin-Acevedo, et al., Next generation of immune checkpoint inhibitors and beyond. Hematol Oncol 14, 45 (2021); each of which publication is incorporated by reference herein in its entirety.

The systems and methods provided herein can be performed at any time during the course of treatment of the subject for the cancer. In some embodiments, the subject has not previously been treated with the immunotherapy. In some embodiments, the subject has not previously been treated with any immunotherapy. The cancer may be early stage, late stage, may comprise a metastatic cancer, a recurrent cancer, or any combination thereof. In some embodiments, the systems and methods provided herein are performed when the subject has not previously been treated for the cancer.

As described herein, the copy number of chromosome or portions thereof can be used to predict a likely benefit or lack of benefit of immunotherapy. In some embodiments, the subject has a loss of copy number of chromosome 9 or the portion thereof and the administered treatment for the cancer is a treatment that is different from the immunotherapy. In some embodiments, the administered treatment for the cancer is a chemotherapy or a combination of immunotherapy and chemotherapy. For example, loss of copy number of chromosome 9 or the portion thereof may indicate lack of benefit of immunotherapy and the treating physician may choose chemotherapy for the patient in the alternative, or the treating physician may choose chemotherapy for the patient in addition to immunotherapy. In other embodiments, wherein the subject does not have a loss of copy number of chromosome 9 or the portion thereof the administered treatment of the cancer is the immunotherapy. The systems and methods can be employed to guide the most beneficial course of treatment and avoid non-beneficial or harmful treatments. Benefit can be measured using various metrics as desired. For example, time-on-treatment, time-to-next-treatment, progression free survival (PFS), disease free survival (DFS), or lifespan may be extended by the administration of the treatment. The treating physician may use the assessment provided by the systems and methods herein to assist in planning a treatment regimen, but the ultimate course of treatment is to be determined by the medical judgement of such treating physician.

The biological sample can be any useful biological sample from the subject such as described herein, including without limitation formalin-fixed paraffin-embedded (FFPE) tissue, fixed tissue, a core needle biopsy, a fine needle aspirate, unstained slides, fresh frozen (FF) tissue, formalin samples, tissue comprised in a solution that preserves nucleic acid or protein molecules, a fresh sample, a malignant fluid, a bodily fluid, a tumor sample, a tissue sample, or any combination thereof. In preferred embodiments, the cells and/or cell free materials derived from the cancer are from a solid tumor. In cases where the biological sample comprises a bodily fluid, the material derived from cancer cells may comprise cell free nucleic acids. For example, cell free nucleic acids shed from cancer cells may be found in blood and blood derivatives such as plasma and serum. In some embodiments, the bodily fluid comprises a malignant fluid, a pleural fluid, a peritoneal fluid, or any combination thereof. The bodily fluid may comprise peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, Cowper's fluid, pre-ejaculatory fluid, female ejaculate, sweat, fecal matter, tears, cyst fluid, pleural fluid, peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyst cavity fluid, umbilical cord blood, or any other useful bodily fluid or combination thereof.

Immunotherapy has been found to be effective across cancer types. In fact, Keytruda® (pembrolizumab) was the first cancer therapy approved by the United States Food and Drug Administration (FDA) based on biomarker status rather than origin of the cancer. Indeed, the systems and methods provided herein can be used to identify a treatment of likely benefit or lack of benefit for multiple cancer types. See, e.g., Example 2. In some embodiments, the cancer comprises an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilms' tumor. In some embodiments, the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumor (GIST), glioblastoma, head and neck squamous carcinoma, leukemia, liver hepatocellular carcinoma, low grade glioma, lung bronchioloalveolar carcinoma (BAC), non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non-epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. In some embodiments, the cancer comprises a head and neck cancer, neuroendocrine cancer, lung cancer, liver cancer, ovarian cancer, or sarcoma. In some embodiments, the cancer comprises breast cancer, bladder cancer, cervical cancer, colon cancer, head and neck cancer, Hodgkin lymphoma, liver cancer, lung cancer, renal cell cancer, melanoma, stomach cancer, rectal cancer, or any solid tumor that exhibits DNA replication errors. Such replication errors can include without limitation mutations, insertions, deletions, translations, fusions, gains or losses, mismatch repair deficiency (MMRd), microsatellite instability (MSI-H), high tumor mutational burden (TMB), or copy number variations (CNV). In some embodiments, the cancer comprises a head and neck cancer or a lung cancer.

The methods and systems provided herein may comprise preparing a molecular profile for the subject based on the copy number of chromosome 9 or the portion thereof. See, e.g., Example 1 for description of comprehensive molecular profiling for cancer. The copy number of chromosome 9 or the portion thereof may be integrated within such comprehensive molecular profiling.

Relatedly, provided herein is a method of generating a molecular profiling report comprising preparing a report summarizing results of performing the methods described above. In some embodiment, the report comprises any identified treatment of likely benefit and/or lack of benefit determined according to the systems and methods provided herein. Such report can be computer generated and can be a printed report or a computer file. In some embodiments, such report accessible via a web portal.

Related to the methods above, provided herein is a system for identifying a treatment for a cancer in a subject, the system comprising: at least one host server; at least one user interface for accessing the at least one host server to access and input data; at least one processor for processing the inputted data; at least one memory coupled to the processor for storing the processed data and instructions for: (1) accessing results of analyzing the biological sample according to the methods provided herein; and (2) determining likely benefit or lack of benefit of an immunotherapy according to the methods provided herein; and at least one display for displaying the likely benefit or lack of benefit of the immunotherapy for treating the cancer. In some embodiments, the at least one display comprises a report comprising the results of analyzing the biological sample and the predicted likely benefit or lack of benefit for treatment of the cancer. In some embodiments, the system further comprises at least one memory coupled to the processor for storing the processed data and instructions for identifying, based on the generated molecular profile according to the methods above, at least one therapy with potential benefit for treatment of the cancer; and at least one display for display thereof. The system may further comprise at least one database comprising references for various biomarker states, data for drug/biomarker associations, or both. The at least one display can be a report provided by the present disclosure.

The specification provides a method of selecting a treatment for a subject who has cancer, the method comprising: obtaining a sample comprising nucleic acid from the cancer; performing an assay to determine a copy number for chromosome 9 or a portion thereof, optionally wherein the portion of chromosome 9 comprises arm 9p, band 9p24, one or more gene located at 9p24, the PD-L1 gene, the PD-L2 gene, the JAK2 gene, the PD-L1 and JAK2 genes, or any useful combination thereof; preparing a molecular profile for the subject the copy number; and selecting a treatment comprising administration of a checkpoint inhibitor therapy, e.g., anti-PD-1 therapy, anti-PD-L1 therapy, anti-CTLA-4 therapy, nivolumab, pembrolizumab, ipilimumab, atezolizumab, avelumab, durvalumab, or cemiplimab, a chemotherapy, or any useful combination thereof, based on the copy number, based on the copy number. In some embodiments, loss of copy indicates lack of benefit from checkpoint inhibitor therapy. The method can be applied to various cancers, including without limitation, breast cancer, bladder cancer, cervical cancer, colon cancer, head and neck cancer, Hodgkin lymphoma, liver cancer, lung cancer, renal cell cancer, melanoma, stomach cancer, rectal cancer, or any solid tumor that exhibits DNA replication errors, e.g., mutations, insertions, deletions, mismatch repair deficiency (MMRd), microsatellite instability (MSI-H), high tumor mutational burden (TMB), copy number variations (CNV). See, e.g., Example 2 herein. In some embodiments, the method further comprises administering the selected treatment to the subject. See, e.g., Example 3.

Report

Related to the methods above, also provided herein is a method of generating a molecular profiling report including preparing a report summarizing the results of performing the methods described herein.

In some embodiments, the report provides the results of the assessment of copy number of chromosome 9, the chromosome band 9p24, and/or one or more genes in the genomic region. In some embodiments, the report provides identification of subjects as potential benefiters and/or non-benefiters based on the assessment. Methods for the identification of benefiters and/or non-benefiters are provided herein. In some embodiments, the report provides treatment recommendations based on the assessment and identification of potential benefiters and/or non-benefiters. In some embodiments, the recommended treatment is an immunotherapy. In some embodiments, the recommended treatment is a combination therapy including immunotherapy and chemotherapy. Alternately, the recommended treatment can exclude immunotherapy. The selection of treatment based on the assessment results are performed as described herein.

The report can be delivered to the treating physician or other caregiver of the subject whose cancer has been profiled. The report can comprise multiple sections of relevant information, including without limitation: 1) a list of the genes in the molecular profile; 2) a description of the molecular profile including copy number of CNV of the genomic regions, genes and/or gene products as determined for the subject; 3) a treatment associated with the molecular profile; and 4) and an indication whether each treatment is likely to benefit the patient, not benefit the patient, or has indeterminate benefit. The list of the genes in the molecular profile can be those presented herein. The description of the molecular profile of the genes as determined for the subject may include such information as the laboratory technique used to assess each biomarker (e.g., NGS, WGS, WES, WTS, RT-PCR, FISH/CISH, PCR, FA/RFLP, CGH, aCGH, etc) as well as the result and criteria used to score each technique. By way of example, the criteria for scoring a CNV (e.g., a CNV of chromosome 9 or a portion thereof) may be a presence (i.e., a copy number that is greater or lower than the “normal” copy number present in a subject who does not have cancer, or statistically identified as present in the general population, typically diploid) or absence (i.e., a copy number that is the same as the “normal” copy number present in a subject who does not have cancer, or statistically identified as present in the general population, typically diploid).

The treatment associated with one or more of the genes and/or gene products in the molecular profile can be determined using a biomarker-treatment association rule set such as in any of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; WO/2016/141169 (Int'l Appl. No. PCT/US2016/020657), published Sep. 9, 2016; and WO2018175501 (Int'l Appl. No. PCT/US2018/023438), published Sep. 27, 2018; Int'l Patent Appl. No. PCT/US2020/012815, filed Jan. 8, 2020; Int'l Patent Appl. No. PCT/US2021/018263, filed Feb. 16, 2021; Int'l Patent Appl. No. PCT/US2019/064078, filed Dec. 2, 2019; Int'l Patent Appl. No. PCT/US2020/035990, filed Jun. 3, 2020; Int'l Patent Appl. No. PCT/US2021/030351, filed Apr. 30, 2021; Int'l Patent Appl. No. PCT/US2021/049966, filed Sep. 10, 2021; each of which publications is incorporated by reference herein in its entirety. The indication whether each treatment is likely to benefit the patient, not benefit the patient, or has indeterminate benefit may be weighted. For example, a potential benefit may be a strong potential benefit or a lesser potential benefit. Such weighting can be based on any appropriate criteria, e.g., the strength of the evidence of the biomarker-treatment association, or the results of the profiling, e.g., a degree of copy number variation.

Various additional components can be added to the report as desired. In some embodiments, the report comprises a list having an indication of whether one or more biomarkers in the molecular profile is associated with an ongoing clinical trial. The report may include identifiers for any such trials, e.g., to facilitate the treating physician's investigation of potential enrollment of the subject in the trial. In some embodiments, the report provides a list of evidence supporting the association of the biomarker in the molecular profile with the reported treatment. The list can contain citations to the evidentiary literature and/or an indication of the strength of the evidence for the particular biomarker-treatment association. In some embodiments, the report comprises a description of the genes in the molecular profile. The description of the genes in the molecular profile can comprise without limitation the biological function and/or various treatment associations. The molecular profiling report can be delivered to the caregiver for the subject, e.g., the oncologist or other treating physician. The caregiver can use the results of the report to guide a treatment regimen for the subject. For example, the caregiver may use one or more treatments indicated as likely benefit in the report to treat the patient. Similarly, the caregiver may avoid treating the patient with one or more treatments indicated as likely lack of benefit in the report.

In some embodiments of the method of identifying at least one therapy of potential benefit, the subject has not previously been treated with the at least one therapy of potential benefit. The cancer may comprise a metastatic cancer, a recurrent cancer, or any combination thereof. In some cases, the cancer is refractory to a prior therapy, including without limitation front-line or standard of care therapy for the cancer. In some embodiments, the cancer is refractory to all known standard of care therapies. In other embodiments, the subject has not previously been treated for the cancer. The method may further comprise administering the at least one therapy of potential benefit to the individual. Progression free survival (PFS), disease free survival (DFS), or lifespan can be extended by the administration.

The report can be computer generated, and can be a printed report, a computer file or both. The report can be made accessible via a secure web portal.

In an aspect, the disclosure provides use of a reagent in carrying out the methods as described herein as described above. In a related aspect, the disclosure provides of a reagent in the manufacture of a reagent or kit for carrying out the methods as described herein. In still another related aspect, the disclosure provides a kit comprising a reagent for carrying out the methods as described herein. The reagent can be any useful and desired reagent. In preferred embodiments, the reagent comprises at least one of a reagent for extracting nucleic acid from a sample, and a reagent for performing next-generation sequencing.

### EXAMPLES

The invention is further described in the following examples, which do not limit the scope as described herein described in the claims.

### Example 1: Next-Generation Profiling

Comprehensive molecular profiling provides a wealth of data concerning the molecular status of patient samples. We have performed such profiling on well over 100,000 tumor patients from practically all cancer lineages using various profiling technologies as described herein. To date, we have tracked the benefit or lack of benefit from treatments in over 20,000 of these patients. Our molecular profiling data can thus be compared to patient benefit to treatments to identify additional biomarker signatures that predict the benefit to various treatments in additional cancer patients. We have applied this “next generation profiling” (NGP) approach to identify biomarker signatures that correlate with patient benefit (including positive, negative, or indeterminate benefit) to various cancer therapeutics.

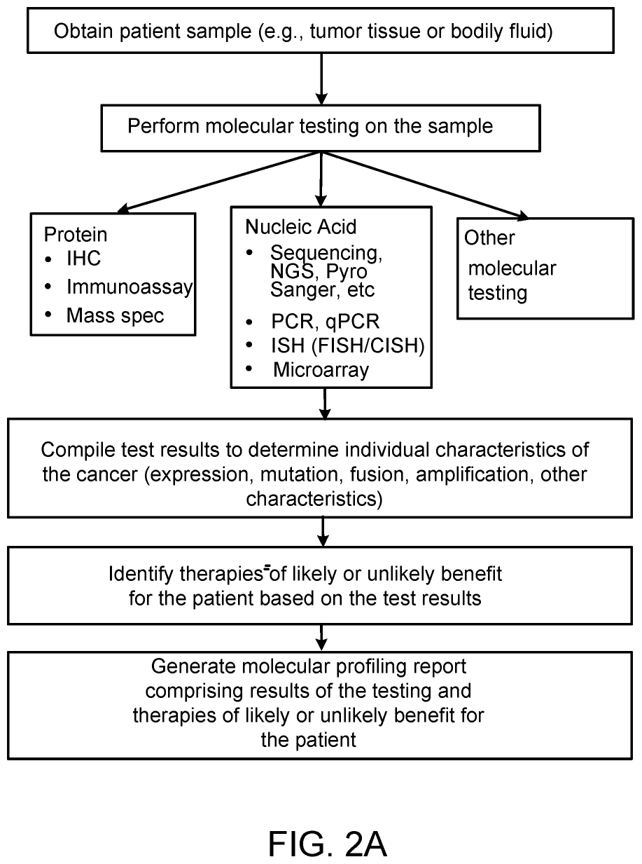

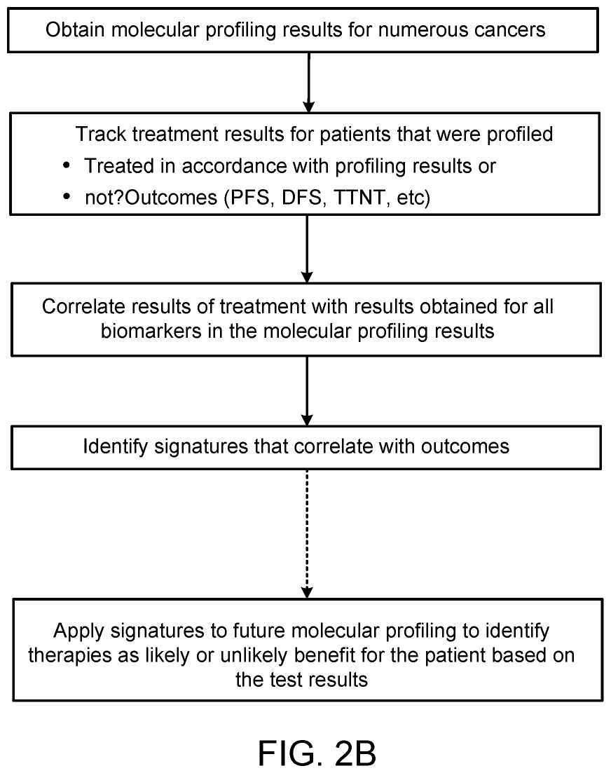

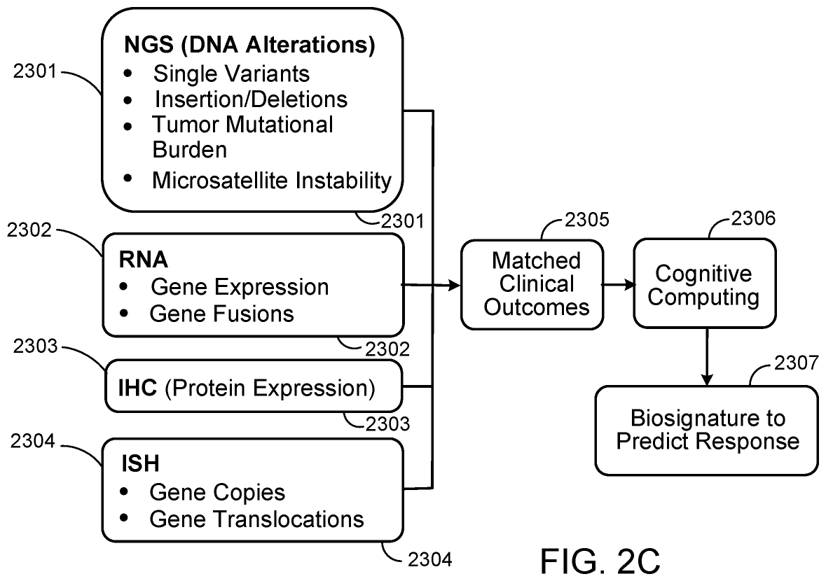

The general approach to NGP is as follows. Over several years we have performed comprehensive molecular profiling of tens of thousands of patients using various molecular profiling techniques. As further outlined in FIG. 2C, these techniques include without limitation next generation sequencing (NGS) of DNA to assess various attributes 2301, gene expression and gene fusion analysis of RNA 2302, IHC analysis of protein expression 2303, and ISH to assess gene copy number and chromosomal aberrations such as translocations 2304. We currently have matched patient clinical outcomes data for over 20,000 patients of various cancer lineages 2305. We use cognitive computing approaches 2306 to correlate the comprehensive molecular profiling results against the actual patient outcomes data for various treatments as desired. Clinical outcome may be determined using the surrogate endpoint time-on-treatment (TOT) or time-to-next-treatment (TTNT or TNT). See, e.g., Roever L (2016) Endpoints in Clinical Trials: Advantages and Limitations. Evidence Based Medicine and Practice 1: e111. doi:10.4172/ebmp.1000e111. The results provide a biosignature comprising a panel of biomarkers 2307, wherein the biosignature is indicative of benefit or lack of benefit from the treatment under investigation. The biosignature can be applied to molecular profiling results for new patients in order to predict benefit from the applicable treatment and thus guide treatment decisions. Such personalized guidance can improve the selection of efficacious treatments and also avoid treatments with lesser clinical benefit, if any.

Table 2 lists numerous biomarkers we have profiled over the past several years. As relevant molecular profiling and patient outcomes are available, any or all of these biomarkers can serve as features to input into the cognitive computing environment to develop a biosignature of interest. The table shows molecular profiling techniques and various biomarkers assessed using those techniques. The listing is non-exhaustive, and data for all of the listed biomarkers will not be available for every patient. It will further be appreciated that various biomarker have been profiled using multiple methods. As a non-limiting example, consider the EGFR gene expressing the Epidermal Growth Factor Receptor (EGFR) protein. As shown in Table 2, expression of EGFR protein has been detected using IHC; EGFR gene amplification, gene rearrangements, mutations and alterations have been detected with ISH, Sanger sequencing, NGS, fragment analysis, and PCR such as qPCR; and EGFR RNA expression has been detected using PCR techniques, e.g., qPCR, and DNA microarray. As a further non-limiting example, molecular profiling results for the presence of the EGFR variant III (EGFRvIII) transcript has been collected using fragment analysis (e.g., RFLP) and sequencing (e.g., NGS).

Table 3 shows exemplary molecular profiles for various tumor lineages. Data from these molecular profiles may be used as the input for NGP in order to identify one or more biosignatures of interest. In the table, the cancer lineage is shown in the column “Tumor Type.” The remaining columns show various biomarkers that can be assessed using the indicated methodology (i.e., immunohistochemistry (IHC), in situ hybridization (ISH), or other techniques). As explained above, the biomarkers are identified using symbols known to those of skill in the art. Under the IHC column, “MMR” refers to the mismatch repair proteins MLH1, MSH2, MSH6, and PMS2, which are each individually assessed using IHC. Under the NGS column “DNA,” “CNA” refers to copy number alteration, which is also referred to herein as copy number variation (CNV). Whole transcriptome sequencing (WTS) is used to assess all RNA transcripts in the specimen. One of skill will appreciate that molecular profiling technologies may be substituted as desired and/or interchangeable. For example, other suitable protein analysis methods can be used instead of IHC (e.g., alternate immunoassay formats), other suitable nucleic acid analysis methods can be used instead of ISH (e.g., that assess copy number and/or rearrangements, translocations and the like), and other suitable nucleic acid analysis methods can be used instead of fragment analysis. Similarly, FISH and CISH are generally interchangeable and the choice may be made based upon probe availability and the like. Tables 4-6 present panels of genomic analysis and genes that have been assessed using Next Generation Sequencing (NGS) analysis of DNA such as genomic DNA. One of skill will appreciate that other nucleic acid analysis methods can be used instead of NGS analysis, e.g., other sequencing (e.g., Sanger), hybridization (e.g., microarray, Nanostring) and/or amplification (e.g., PCR based) methods. The biomarkers listed in Tables 7-8 can be assessed by RNA sequencing, such as WTS. Using WTS, any fusions, splice variants, or the like can be detected. Tables 7-8 list biomarkers with commonly detected alterations in cancer.

Nucleic acid analysis may be performed to assess various aspects of a gene. For example, nucleic acid analysis can include, but is not limited to, mutational analysis, fusion analysis, variant analysis, splice variants, SNP analysis and gene copy number/amplification. Such analysis can be performed using any number of techniques described herein or known in the art, including without limitation sequencing (e.g., Sanger, Next Generation, pyrosequencing), PCR, variants of PCR such as RT-PCR, fragment analysis, and the like. NGS techniques may be used to detect mutations, fusions, variants and copy number of multiple genes in a single assay. Unless otherwise stated or obvious in context, a “mutation” as used herein may comprise any change in a gene or genome as compared to wild type, including without limitation a mutation, polymorphism, deletion, insertion, indels (i.e., insertions or deletions), substitution, translocation, fusion, break, duplication, amplification, repeat, or copy number variation. Different analyses may be available for different genomic alterations and/or sets of genes. For example, Table 4 lists attributes of genomic stability that can be measured with NGS, Table 5 lists various genes that may be assessed for point mutations and indels, Table 6 lists various genes that may be assessed for point mutations, indels and copy number variations, Table 7 lists various genes that may be assessed for gene fusions via RNA analysis, e.g., via WTS, and similarly Table 8 lists genes that can be assessed for transcript variants via RNA. Molecular profiling results for additional genes can be used to identify an NGP biosignature as such data is available.

As noted in Table 2, NGS can be used for whole exome sequencing (WES), whole genome sequencing (WGS), and/or whole transcriptome sequencing (WTS). Such methods can allow for simultaneous analysis of all substantially all or all exons in genomic DNA, simultaneous analysis of all substantially all or all genomic DNA, and simultaneous analysis of substantially all or all mRNA transcripts. Molecular profiling can employ any of these techniques as desired.

TABLE 2
Molecular Profiling Biomarkers

| Technique | Biomarkers |
|---|---|
| IHC | ABL1, ACPP (PAP), Actin (ACTA), ADA, AFP, AKT1, ALK, ALPP |
| | (PLAP-1), APC, AR, ASNS, ATM, BAP1, BCL2, BCRP, BRAF, |
| | BRCA1, BRCA2, CA19-9, CALCA, CCND1 (BCL1), CCR7, CD19, |
| | CD276, CD3, CD33, CD52, CD80, CD86, CD8A, CDH1 (ECAD), |
| | CDW52, CEACAM5 (CEA; CD66e), CES2, CHGA (CGA), CK 14, CK |
| | 17, CK 5/6, CK1, CK10, CK14, CK15, CK16, CK19, CK2, CK3, CK4, |
| | CK5, CK6, CK7, CK8, COX2, CSF1R, CTL4A, CTLA4, CTNNB1, |
| | Cytokeratin, DCK, DES, DNMT1, EGFR, EGFR H-score, ERBB2 |
| | (HER2), ERBB4 (HER4), ERCC1, ERCC3, ESR1 (ER), F8 (FACTOR8), |
| | FBXW7, FGFR1, FGFR2, FLT3, FOLR2, GART, GNA11, GNAQ, |
| | GNAS, Granzyme A, Granzyme B, GSTP1, HDAC1, HIF1A, HNF1A, |
| | HPL, HRAS, HSP90AA1 (HSPCA), IDH1, IDO1, IL2, IL2RA (CD25), |
| | JAK2, JAK3, KDR (VEGFR2), KI67, KIT (cKIT), KLK3 (PSA), KRAS, |
| | KRT20 (CK20), KRT7 (CK7), KRT8 (CYK8), LAG-3, MAGE-A, MAP |
| | KINASE PROTEIN (MAPK1/3), MDM2, MET (cMET), MGMT, |
| | MLH1, MPL, MRP1, MS4A1 (CD20), MSH2, MSH4, MSH6, MSI, |
| | MTAP, MUC1, MUC16, NFKB1, NFKB1A, NFKB2, NGF, NOTCH1, |
| | NPM1, NRAS, NY-ESO-1, ODC1 (ODC), OGFR, p16, p95, PARP-1, |
| | PBRM1, PD-1, PDGF, PDGFC, PDGFR, PDGFRA, PDGFRA |
| | (PDGFR2), PDGFRB (PDGFR1), PD-L1, PD-L2, PGR (PR), PIK3CA, |
| | PIP, PMEL, PMS2, POLA1 (POLA), PR, PTEN, PTGS2 (COX2), |
| | PTPN11, RAF1, RARA (RAR), RB1, RET, RHOH, ROS1, RRM1, RXR, |
| | RXRB, S100B, SETD2, SMAD4, SMARCB1, SMO, SPARC, SST, |
| | SSTR1, STK11, SYP, TAG-72, TIM-3, TK1, TLE3, TNF, TOP1 |
| | (TOPO1), TOP2A (TOP2), TOP2B (TOPO2B), TP, TP53 (p53), |
| | TRKA/B/C, TS, TUBB3, TXNRD1, TYMP (PDECGF), TYMS (TS), |
| | VDR, VEGFA (VEGF), VHL, XDH, ZAP70 |
| ISH (CISH/FISH) | 1p19q, ALK, EML4-ALK, EGFR, ERCC1, HER2, HPV (human |
| | papilloma virus), MDM2, MET, MYC, PIK3CA, ROS1, TOP2A, |
| | chromosome 17, chromosome 12 |
| Pyrosequencing | MGMT promoter methylation |
| Sanger sequencing | BRAF, EGFR, GNA11, GNAQ, HRAS, IDH2, KIT, KRAS, NRAS, |
| | PIK3CA |
| NGS | See genes and types of testing in Tables 3-8, MSI, TMB |
| | Whole exome (e.g., via WES) |
| | Whole genome (e.g., via WGS) |
| | Whole transcriptome (e.g., via WTS) |
| Fragment Analysis | ALK, EML4-ALK, EGFR Variant III, HER2 exon 20, ROS1, MSI |
| PCR | ALK, AREG, BRAF, BRCA1, EGFR, EML4, ERBB3, ERCC1, EREG, |
| | hENT-1, HSP90AA1, IGF-1R, KRAS, MMR, p16, p21, p27, PARP-1, |
| | PGP (MDR-1), PIK3CA, RRM1, TLE3, TOPO1, TOPO2A, TS, TUBB3 |
| Microarray | ABCC1, ABCG2, ADA, AR, ASNS, BCL2, BIRC5, BRCA1, BRCA2, |
| | CD33, CD52, CDA, CES2, DCK, DHFR, DNMT1, DNMT3A, |
| | DNMT3B, ECGF1, EGFR, EPHA2, ERBB2, ERCC1, ERCC3, ESR1, |
| | FLT1, FOLR2, FYN, GART, GNRH1, GSTP1, HCK, HDAC1, HIF1A, |
| | HSP90AA1 (HSPCA), IL2RA, HSP90AA1, KDR, KIT, LCK, LYN, |
| | MGMT, MLH1, MS4A1, MSH2, NFKB1, NFKB2, OGFR, PDGFC, |
| | PDGFRA, PDGFRB, PGR, POLA1, PTEN, PTGS2, RAF1, RARA, |
| | RRM1, RRM2, RRM2B, RXRB, RXRG, SPARC, SRC, SSTR1, SSTR2, |
| | SSTR3, SSTR4, SSTR5, TK1, TNF, TOP1, TOP2A, TOP2B, TXNRD1, |
| | TYMS, VDR, VEGFA, VHL, YES1, ZAP70 |

TABLE 3
Molecular Profiles

| | | Next-Generation | | Whole | |
|---|---|---|---|---|---|
| | | Sequencing (NGS) | | Transcriptome | |
| | | | Genomic | Sequencing | |
| | | | Signatures | (WTS) | |
| Tumor Type | IHC | DNA | (DNA) | RNA | Other |
| Bladder | MMR, PD-L1 | Mutation, | MSI, TMB | Fusion Analysis | |
| | | CNA | | | |
| Breast | AR, ER, Her2/Neu, | Mutation, | MSI, TMB | Fusion Analysis | Her2, TOP2A |
| | MMR, PD-L1, PR, | CNA | | | (CISH) |
| | PTEN | | | | |
| Cancer of Unknown | MMR, PD-L1 | Mutation, | MSI, TMB | Fusion Analysis | |
| Primary | | CNA | | | |
| Cervical | ER, MMR, PD-L1, | Mutation, | MSI, TMB | | |
| | PR, TRKA/B/C | CNA | | | |
| Cholangiocarcinoma/ | Her2/Neu, MMR, | Mutation, | MSI, TMB | Fusion Analysis | Her2 (CISH) |
| Hepatobiliary | PD-L1 | CNA | | | |
| Colorectal and Small | Her2/Neu, MMR, | Mutation, | MSI, TMB | Fusion Analysis | |
| Intestinal | PD-L1, PTEN | CNA | | | |
| Endometrial | ER, MMR, PD-L1, | Mutation, | MSI, TMB | Fusion Analysis | |
| | PR, PTEN | CNA | | | |
| Esophageal | Her2/Neu, MMR, | Mutation, | MSI, TMB | | |
| | PD-L1, TRKA/B/C | CNA | | | |
| Gastric/GEJ | Her2/Neu, MMR, | Mutation, | MSI, TMB | | Her2 (CISH) |
| | PD-L1, TRKA/B/C | CNA | | | |
| GIST | MMR, PD-L1, | Mutation, | MSI, TMB | | |
| | PTEN, TRKA/B/C | CNA | | | |
| Glioma | MMR, PD-L1 | Mutation, | MSI, TMB | Fusion Analysis | MGMT |
| | | CNA | | | Methylation |
| | | | | | (Pyrosequencing) |
| Head & Neck | MMR, p16, PD-L1, | Mutation, | MSI, TMB | | HPV (CISH), |
| | TRKA/B/C | CNA | | | reflex to confirm |
| | | | | | p16 result |
| Kidney | MMR, PD-L1, | Mutation, | MSI, TMB | | |
| | TRKA/B/C | CNA | | | |
| Melanoma | MMR, PD-L1, | Mutation, | MSI, TMB | | |
| | TRKA/B/C | CNA | | | |
| Merkel Cell | MMR, PD-L1, | Mutation, | MSI, TMB | | |
| | TRKA/B/C | CNA | | | |
| Neuroendocrine/Small | MMR, PD-L1, | Mutation, | MSI, TMB | | |
| Cell Lung | TRKA/B/C | CNA | | | |
| Non-Small Cell Lung | ALK, MMR, PD- | Mutation, | MSI, TMB | Fusion Analysis | |
| | L1, PTEN | CNA | | | |
| Ovarian | ER, MMR, PD-L1, | Mutation, | MSI, TMB | | |
| | PR, TRKA/B/C | CNA | | | |
| Pancreatic | MMR, PD-L1 | Mutation, | MSI, TMB | Fusion Analysis | |
| | | CNA | | | |
| Prostate | AR, MMR, PD-L1 | Mutation, | MSI, TMB | Fusion Analysis | |
| | | CNA | | | |
| Salivary Gland | AR, Her2/Neu, | Mutation, | MSI, TMB | Fusion Analysis | |
| | MMR, PD-L1 | CNA | | | |
| Sarcoma | MMR, PD-L1 | Mutation, | MSI, TMB | Fusion Analysis | |
| | | CNA | | | |
| Thyroid | MMR, PD-L1 | Mutation, | MSI, TMB | Fusion Analysis | |
| | | CNA | | | |
| Uterine Serous | ER, Her2/Neu, | Mutation, | MSI, TMB | | Her2 (CISH) |
| | MMR, PD-L1, PR, | CNA | | | |
| | PTEN, TRKA/B/C | | | | |
| Vulvar Cancer (SCC) | ER, MMR, PD-L1 | Mutation, | MSI, TMB | | |
| | (22c3), PR, TRK | CNA | | | |
| | A/B/C | | | | |
| Other Tumors | MMR, PD-L1, | Mutation, | MSI, TMB | | |
| | TRKA/B/C | CNA | | | |

TABLE 4
Genomic Stability Testing (DNA)

| | |
|---|---|
| Microsatellite Instability (MSI) | Tumor Mutational Burden (TMB) |

TABLE 5
Point Mutations and Indels (DNA)

ABI1
ABL1
ACKR3
AKT1
AMER1 (FAM123B)
AR
ARAF
ATP2B3
ATRX
BCL11B
BCL2
BCL2L2
BCOR
BCORL1
BRD3
BRD4
BTG1
BTK
C15orf65
CBLC
CD79B
CDH1
CDK12
CDKN2B
CDKN2C
CEBPA
CHCHD7
CNOT3
COL1A1
COX6C
CRLF2
DDB2
DDIT3
DNM2
DNMT3A
EIF4A2
ELF4
ELN
ERCC1
ETV4
FAM46C
FANCF
FEV
FOXL2
FOXO3
FOXO4
FSTL3
GATA1
GATA2
GNA11
GPC3
HEY1
HIST1H3B
HIST1H4I
HLF
HMGN2P46
HNF1A
HOXA11
HOXA13
HOXA9
HOXC11
HOXC13
HOXD11
HOXD13
HRAS
IKBKE
INHBA
IRS2
JUN
KAT6A (MYST3)
KAT6B
KCNJ5
KDM5C
KDM6A
KDSR
KLF4
KLK2
LASP1
LMO1
LMO2
MAFB
MAX
MECOM
MED12
MKL1
MLLT11
MN1
MPL
MSN
MTCP1
MUC1
MUTYH
MYCL (MYCL1)
NBN
NDRG1
NKX2-1
NONO
NOTCH1
NRAS
NUMA1
NUTM2B
OLIG2
OMD
P2RY8
PAFAH1B2
PAK3
PATZ1
PAX8
PDE4DIP
PHF6
PHOX2B
PIK3CG
PLAG1
PMS1
POU5F1
PPP2R1A
PRF1
PRKDC
RAD21
RECQL4
RHOH
RNF213
RPL10
SEPT5
SEPT6
SFPQ
SLC45A3
SMARCA4
SOCS1
SOX2
SPOP
SRC
SSX1
STAG2
TAL1
TAL2
TBL1XR1
TCEA1
TCL1A
TERT
TFE3
TFPT
THRAP3
TLX3
TMPRSS2
UBR5
VHL
WAS
ZBTB16
ZRSR2

TABLE 6
Point Mutations, Indels and Copy Number Variations (DNA)

ABL2
ACSL3
ACSL6
ADGRA2
AFDN
AFF1
AFF3
AFF4
AKAP9
AKT2
AKT3
ALDH2
ALK
APC
ARFRP1
ARHGAP26
ARHGEF12
ARID1A
ARID2
ARNT
ASPSCR1
ASXL1
ATF1
ATIC
ATM
ATP1A1
ATR
AURKA
AURKB
AXIN1
AXL
BAP1
BARD1
BCL10
BCL11A
BCL2L11
BCL3
BCL6
BCL7A
BCL9
BCR
BIRC3
BLM
BMPR1A
BRAF
BRCA1
BRCA2
BRIP1
BUB1B
CACNA1D
CALR
CAMTA1
CANT1
CARD11
CARS
CASP8
CBFA2T3
CBFB
CBL
CBLB
CCDC6
CCNB1IP1
CCND1
CCND2
CCND3
CCNE1
CD274 (PDL1)
CD74
CD79A
CDC73
CDH11
CDK4
CDK6
CDK8
CDKN1B
CDKN2A
CDX2
CHEK1
CHEK2
CHIC2
CHN1
CIC
CIITA
CLP1
CLTC
CLTCL1
CNBP
CNTRL
COPB1
CREB1
CREB3L1
CREB3L2
CREBBP
CRKL
CRTC1
CRTC3
CSF1R
CSF3R
CTCF
CTLA4
CTNNA1
CTNNB1
CYLD
CYP2D6
DAXX
DDR2
DDX10
DDX5
DDX6
DEK
DICER1
DOT1L
EBF1
ECT2L
EGFR
ELK4
ELL
EML4
EMSY
EP300
EPHA3
EPHA5
EPHB1
EPS15
ERBB2 (HER2/NEU)
ERBB3 (HER3)
ERBB4 (HER4)
ERC1
ERCC2
ERCC3
ERCC4
ERCC5
ERG
ESR1
ETV1
ETV5
ETV6
EWSR1
EXT1
EXT2
EZH2
EZR
FANCA
FANCC
FANCD2
FANCE
FANCG
FANCL
FAS
FBXO11
FBXW7
FCRL4
FGF10
FGF14
FGF19
FGF23
FGF3
FGF4
FGF6
FGFR1
FGFR1OP
FGFR2
FGFR3
FGFR4
FH
FHIT
FIP1L1
FLCN
FLI1
FLT1
FLT3
FLT4
FNBP1
FOXA1
FOXO1
FOXP1
FUBP1
FUS
GAS7
GATA3
GID4 (C17orf39)
GMPS
GNA13
GNAQ
GNAS
GOLGA5
GOPC
GPHN
GRIN2A
GSK3B
H3F3A
H3F3B
HERPUD1
HGF
HIP1
HMGA1
HMGA2
HNRNPA2B1
HOOK3
HSP90AA1
HSP90AB1
IDH1
IDH2
IGF1R
IKZF1
IL2
IL21R
IL6ST
IL7R
IRF4
ITK
JAK1
JAK2
JAK3
JAZF1
KDM5A
KDR (VEGFR2)
KEAP1
KIAA1549
KIF5B
KIT
KLHL6
KMT2A (MLL)
KMT2C (MLL3)
KMT2D (MLL2)
KNL1
KRAS
KTN1
LCK
LCP1
LGR5
LHFPL6
LIFR
LPP
LRIG3
LRP1B
LYL1
MAF
MALT1
MAML2
MAP2K1 (MEK1)
MAP2K2 (MEK2)
MAP2K4
MAP3K1
MCL1
MDM2
MDM4
MDS2
MEF2B
MEN1
MET
MITF
MLF1
MLH1
MLLT1
MLLT10
MLLT3
MLLT6
MNX1
MRE11
MSH2
MSH6
MSI2
MTOR
MYB
MYC
MYCN
MYD88
MYH11
MYH9
NACA
NCKIPSD
NCOA1
NCOA2
NCOA4
NF1
NF2
NFE2L2
NFIB
NFKB2
NFKBIA
NIN
NOTCH2
NPM1
NSD1
NSD2
NSD3
NT5C2
NTRK1
NTRK2
NTRK3
NUP214
NUP93
NUP98
NUTM1
PALB2
PAX3
PAX5
PAX7
PBRM1
PBX1
PCM1
PCSK7
PDCD1 (PD1)
PDCD1LG2 (PDL2)
PDGFB
PDGFRA
PDGFRB
PDK1
PER1
PICALM
PIK3CA
PIK3R1
PIK3R2
PIM1
PML
PMS2
POLE
POT1
POU2AF1
PPARG
PRCC
PRDM1
PRDM16
PRKAR1A
PRRX1
PSIP1
PTCH1
PTEN
PTPN11
PTPRC
RABEP1
RAC1
RAD50
RAD51
RAD51B
RAF1
RALGDS
RANBP17
RAPIGDS1
RARA
RB1
RBM15
REL
RET
RICTOR
RMI2
RNF43
ROS1
RPL22
RPL5
RPN1
RPTOR
RUNX1
RUNX1T1
SBDS
SDC4
SDHAF2
SDHB
SDHC
SDHD
SEPT9
SET
SETBP1
SETD2
SF3B1
SH2B3
SH3GL1
SLC34A2
SMAD2
SMAD4
SMARCB1
SMARCE1
SMO
SNX29
SOX10
SPECC1
SPEN
SRGAP3
SRSF2
SRSF3
SS18
SS18L1
STAT3
STAT4
STAT5B
STIL
STK11
SUFU
SUZ12
SYK
TAF15
TCF12
TCF3
TCF7L2
TET1
TET2
TFEB
TFG
TFRC
TGFBR2
TLX1
TNFAIP3
TNFRSF14
TNFRSF17
TOP1
TP53
TPM3
TPM4
TPR
TRAF7
TRIM26
TRIM27
TRIM33
TRIP11
TRRAP
TSC1
TSC2
TSHR
TTL
U2AF1
USP6
VEGFA
VEGFB
VTI1A
WDCP
WIFI
WISP3
WRN
WT1
WWTR1
XPA
XPC
XPO1
YWHAE
ZMYM2
ZNF217
ZNF331
ZNF384
ZNF521
ZNF703

TABLE 7
Gene Fusions (RNA)

AKT3
ALK
ARHGAP26
AXL
BRAF
BRD3
BRD4
EGFR
ERG
ESR1
ETV1
ETV4
ETV5
ETV6
EWSR1
FGFR1
FGFR2
FGFR3
FGR
INSR
MAML2
MAST1
MAST2
MET
MSMB
MUSK
MYB
NOTCH1
NOTCH2
NRG1
NTRK1
NTRK2
NTRK3
NUMBL
NUTM1
PDGFRA
PDGFRB
PIK3CA
PKN1
PPARG
PRKCA
PRKCB
RAF1
RELA
RET
ROS1
RSPO2
RSPO3
TERT
TFE3
TFEB
THADA
TMPRSS2

TABLE 8
Variant Transcripts

| | | |
|---|---|---|
| AR-V7 | EGFR vIII | MET Exon 14 Skipping |

Abbreviations used in this Example and throughout the specification, e.g., IHC: immunohistochemistry; ISH: in situ hybridization; CISH: colorimetric in situ hybridization; FISH: fluorescent in situ hybridization; NGS: next generation sequencing; PCR: polymerase chain reaction; CNA: copy number alteration; CNV: copy number variation; MSI: microsatellite instability; TMB: tumor mutational burden.

### Example 2: Prediction of Benefit of Immunotherapy in Head and Neck and Other Cancers

In this Example, we used comprehensive molecular profiling data (see, e.g., Example 1 above; Tables 5-12 of WO/2018/175501 (based on International Application No. PCT/US2018/023438 filed 20 Mar. 2018), as well as WO/2015/116868 (based on International Application No. PCT/US2015/013618, filed 29 Jan. 2015), WO/2017/053915 (based on International Application No. PCT/US2016/053614, filed 24 Sep. 2016), and WO/2016/141169 (based on International Application No. PCT/US2016/020657, filed 3 Mar. 2016)) to identify a biosignature for predicting benefit or lack of benefit from immunotherapy for treatment of cancer.

Immune checkpoint therapy is also typically prescribed upon indication from a companion diagnostic (e.g., to confirm expression of the target protein), but it is not always efficacious. For example, the response rate to pembrolizumab may be less than 50% even in patients pre-selected for expression of PD-L1 on at least 50% of tumor cells. See, e.g., Reck, M., et al., Pembrolizumab versus Chemotherapy for PD-L1-Positive Non-Small-Cell Lung Cancer. N Engl J Med 2016; 375:1823-1833. And in some cases, checkpoint inhibitor therapy may exacerbate hyperprogressive disease characterized by acceleration of tumor growth during treatment. See, e.g., Ferrara, R et al., Hyperprogressive Disease in Patients With Advanced Non-Small Cell Lung Cancer Treated With PD-1/PD-L1 Inhibitors or With Single-Agent Chemotherapy. JAMA Oncol. 2018 Nov. 1; 4(11):1543-1552. Combined with the high costs and potential for adverse reactions to checkpoint inhibitor therapy, we set out to improve identification of those patients more likely to benefit from such therapies.

The patient cohort comprised patients with head and neck cancer whose tumors we profiled as described above. The patients were all Human papillomavirus-negative (HPV−) as determined by p16 negativity. The patients had been treated with immunotherapy (IO), either pembrolizumab or nivolumab, after the molecular profiling was performed. Overall survival (OS) was measured as first immunotherapy to last contact. Genomic alterations were determined by next-generation sequencing (NGS).

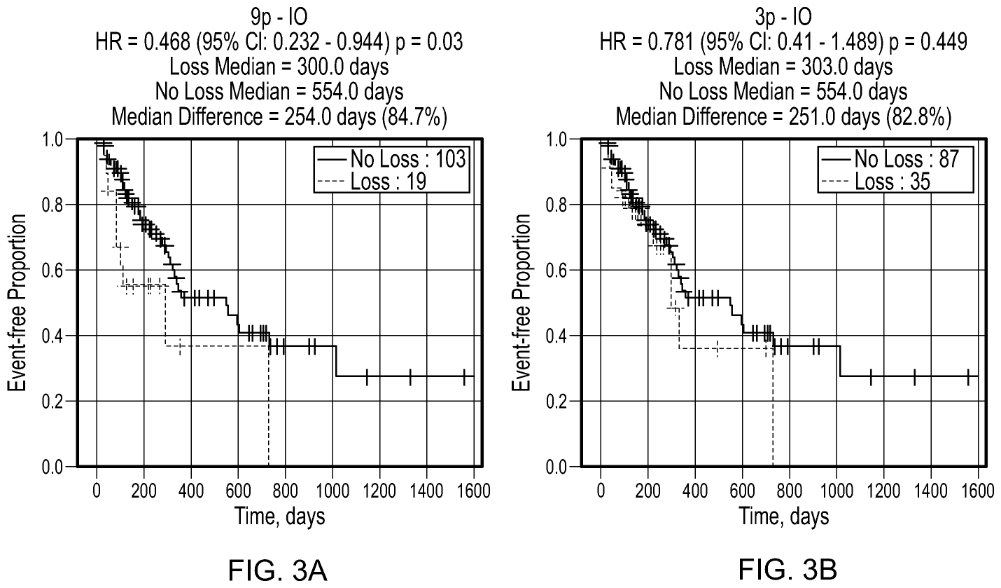

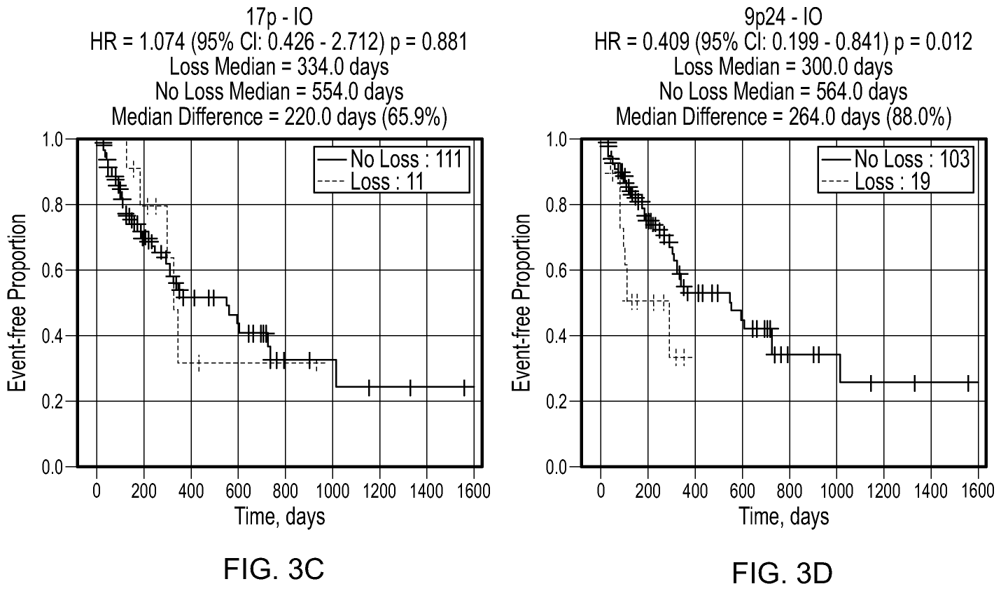

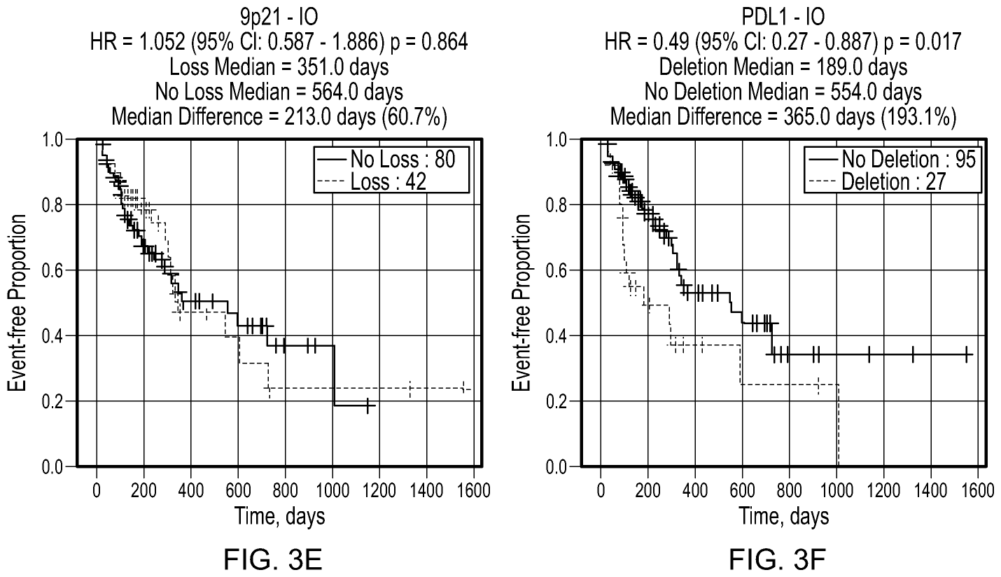

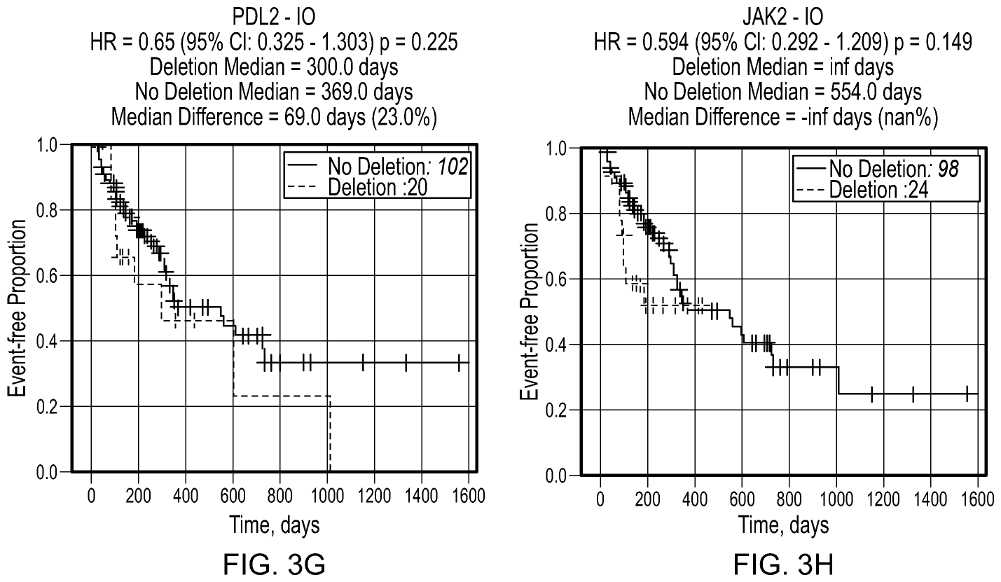

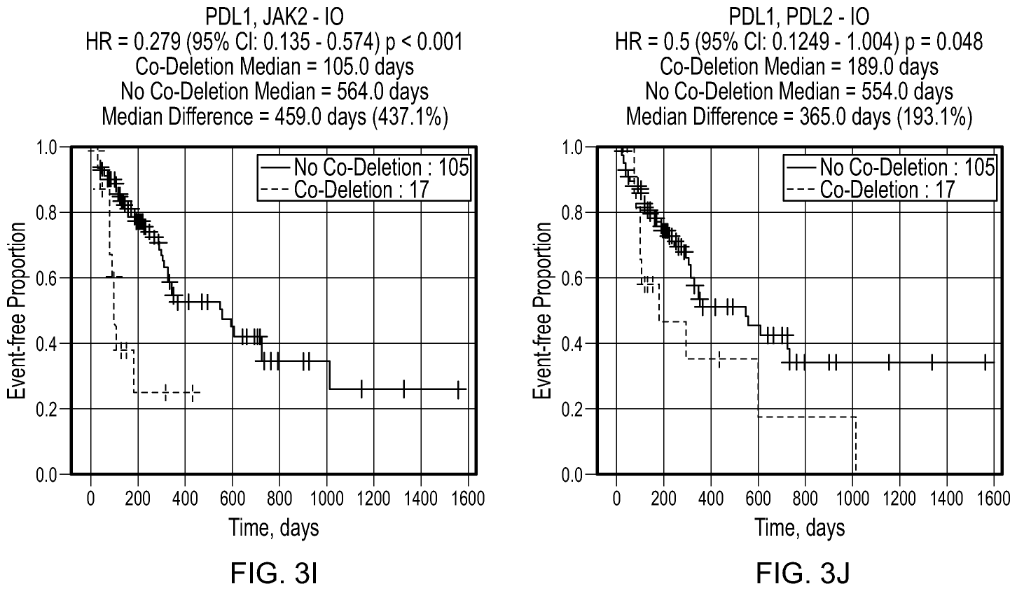

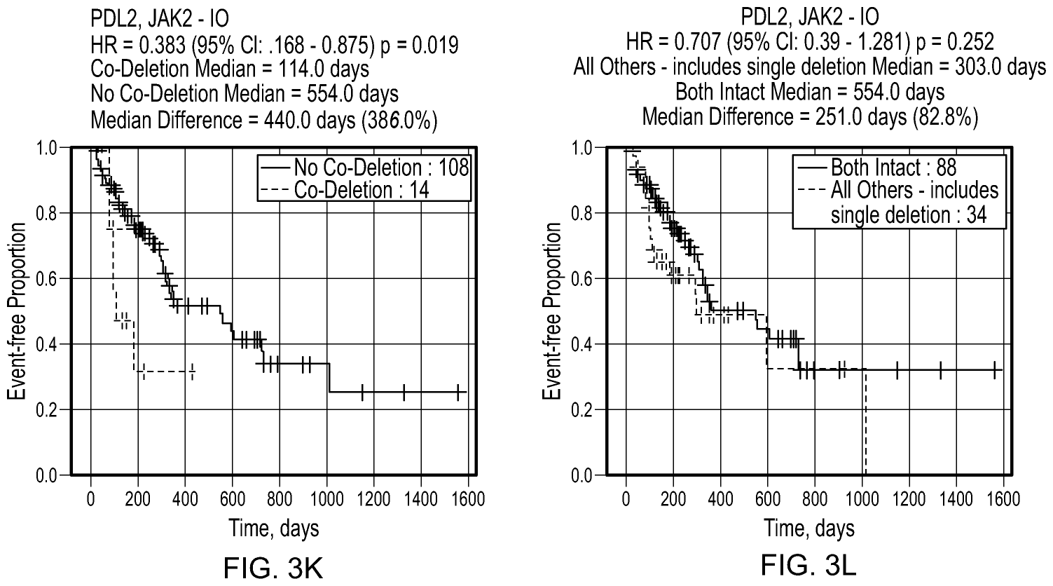

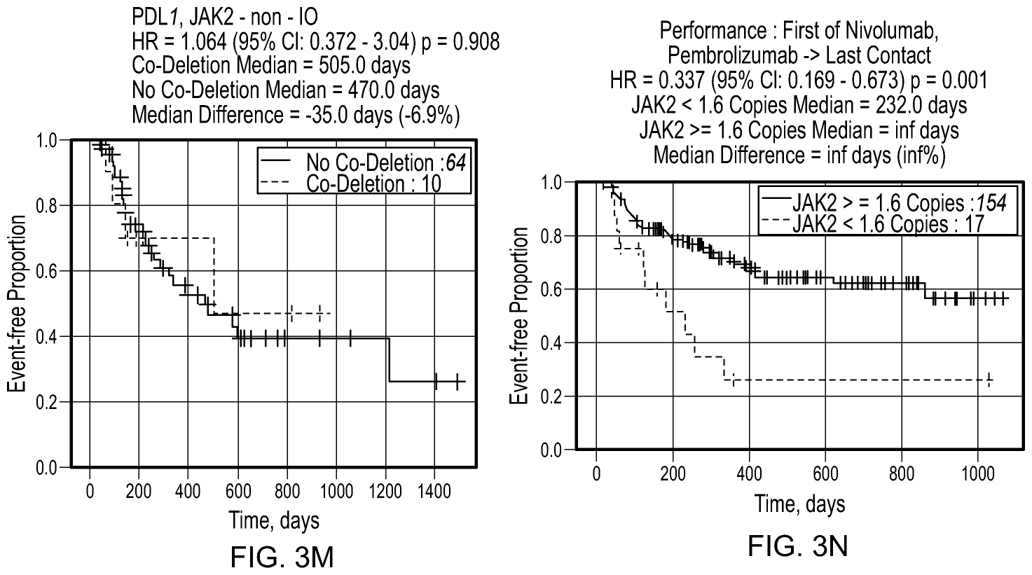

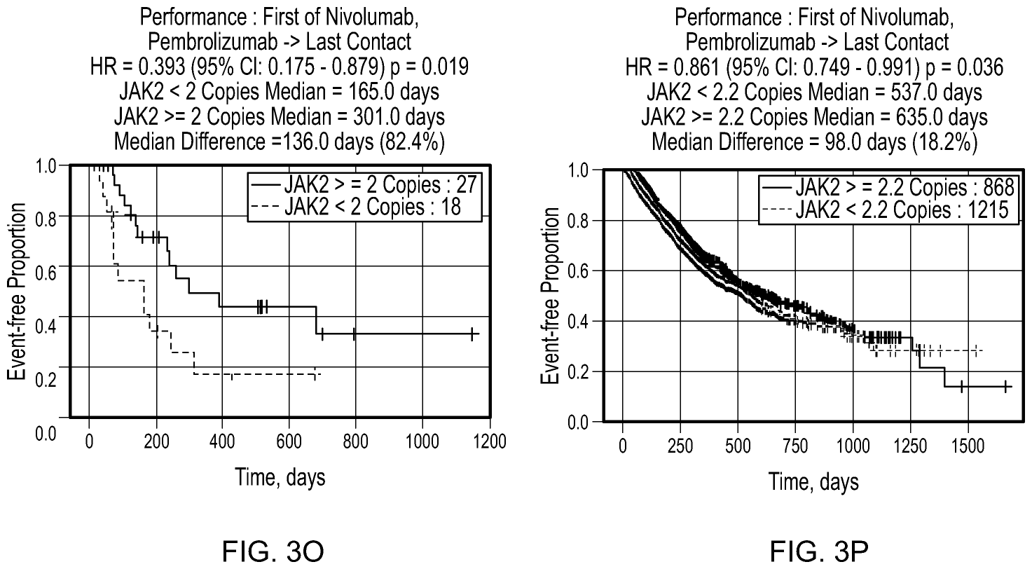

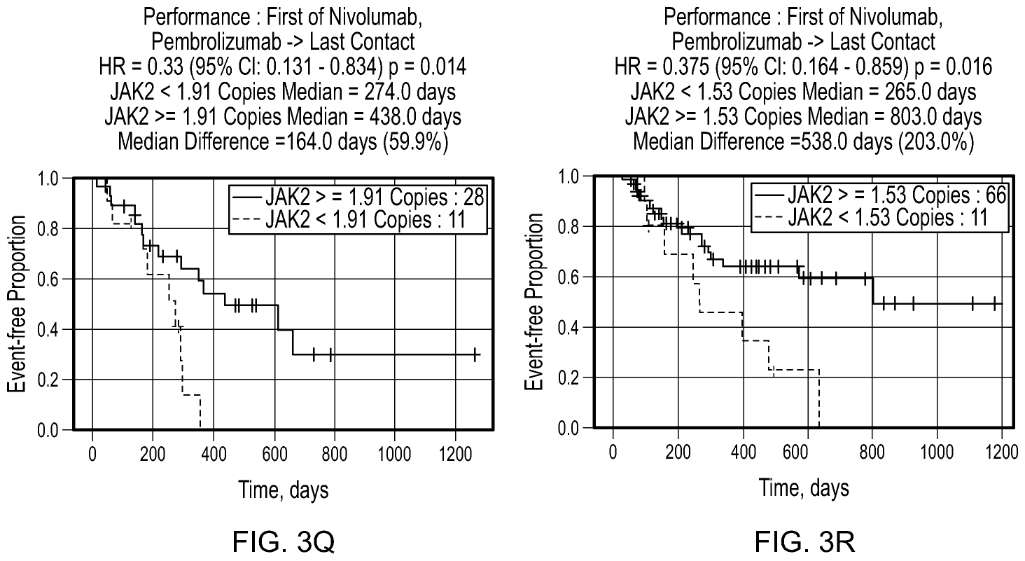

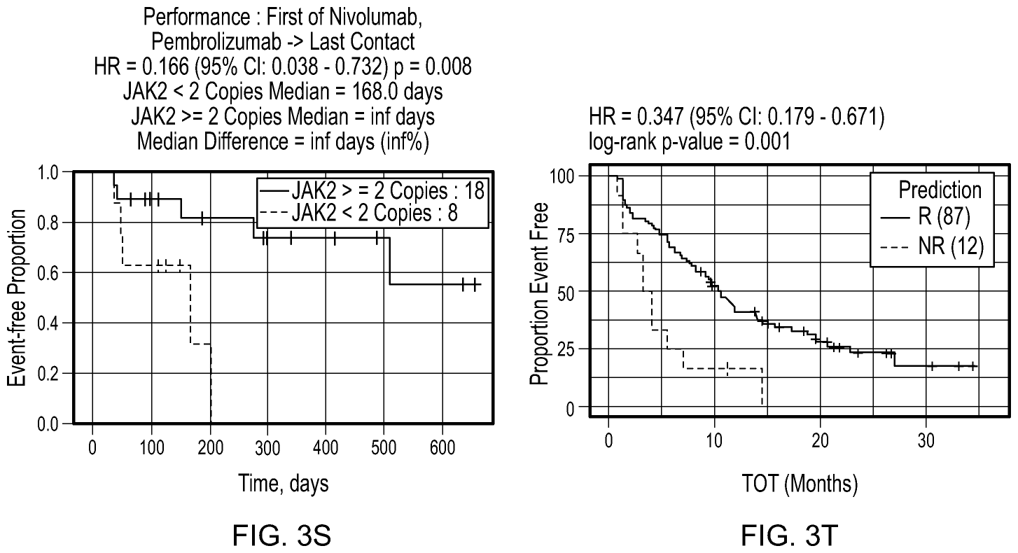

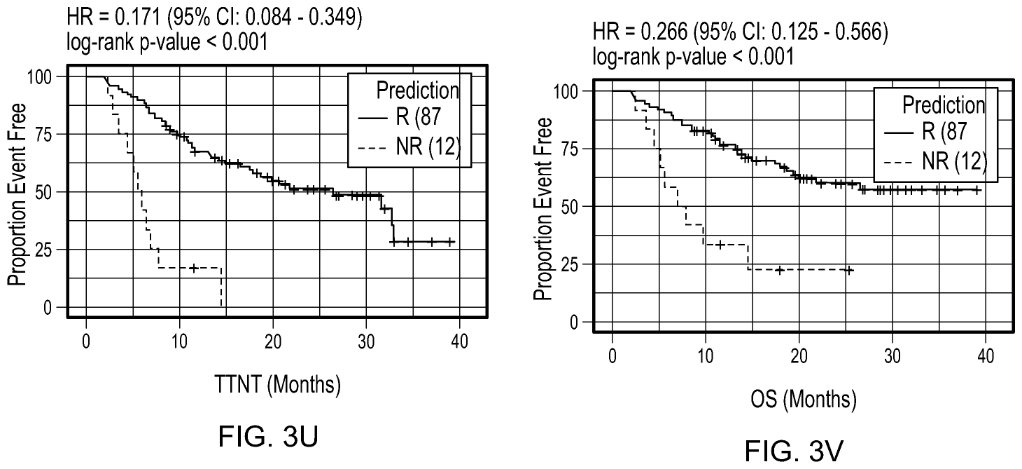

We first examined genomic alterations that correlated with OS. Loss at chromosomal arm 9p was found to correlate with OS. See FIG. 3A, which shows a Kaplan-Meier survival curve after treatment with IO in patients with loss or no loss at 9p, as indicated in the plot. The p-value was significant at 0.03.

No such correlation between immunotherapy and OS was found generally with chromosomal abnormalities. For example, FIGS. 3B and 3C show that loss at 3p or 17p, respectively, did not correlate with OS.

We next examined whether various bands on 9p showed the same signal as 9p itself. The signal was found to localize to band 9p24. FIGS. 3D and 3E show Kaplan-Meier plots as in FIG. 3A, except that loss was determined at bands 9p24 (p-value=0.012) or 9p21 (p-value=0.864), respectively. We then assessed certain genes on 9p24. FIGS. 3F-3H show Kaplan-Meier plots as above, except that loss was determined at genes PD-L1 (FIG. 3F; p-value=0.017), PD-L2 (FIG. 3G; p-value=0.225) or JAK2 (FIG. 3F; p-value=0.149). Loss of gene copies of PD-L1 was most significantly correlated with OS in the head and neck cancer patients treated with immunotherapy.

Further details of the genes are shown in Table 9. The table lists common gene symbols, name, and Gene ID from the Entrez gene browser made available by the National Center for Biotechnology Information, U.S. National Library of Medicine, U.S. National Institute of Health (see ncbi.nlm.nih.gov/gene).

TABLE 9
Immunotherapy response predictor features

| | | Entrez |
|---|---|---|
| Symbol/s | Name | Gene ID |
| CD274, PD-L1, PDL1, | CD274 Antigen, Programmed | 29126 |
| B7H1 | Cell Death 1 Ligand 1 | |
| CD273, PD-L2, PDL2, | Programmed Cell Death 1 | 80380 |
| B7DC, PDCD1LG2 | Ligand 2 | |
| JAK2 | Janus Kinase 2 | 3713 |

We next determined whether co-deletion of certain genes would further stratify patients by OS. FIGS. 3I-3K show Kaplan-Meier plots as above, except that the patient cohort was split by co-deletion or not of PD-L1+JAK2 (FIG. 31; p-value=0.001), PD-L1+PD-L2 (FIG. 3J; p-value=0.048) or PD-L2+JAK2 (FIG. 3K; p-value=0.019). In all cases, the analysis by co-deletion was statistically significant, and PD-L1+JAK2 provided the best separation of all scenarios with hazard ratio (HR)=0.279 and p-value=0.001. In contrast, no significant difference was observed when patients were split depending on whether both PD-L2+JAK2 were intact or not. See FIG. 3L.

The above data indicates that copy number of PD-L1+JAK2 can stratify patients according to survival when treated with immunotherapy. To ask whether this combination is a prognostic indicator, i.e., identifies patients with better outcome regardless of treatment, we examined whether co-deletion of PD-L1+JAK2 predict response of head and neck cancer patients to chemotherapy. FIG. 3M shows that this combination was not able to predict chemotherapy response. This data indicates that analysis of 9p24 and genes located there are true predictors of response to pembrolizumab or nivolumab. Thus, we provide a method of identifying head and neck cancer patients that are more likely to respond to such immunotherapy.

Pembrolizumab and nivolumab are monoclonal antibodies against PD-1 and are used to treat cancer that overexpress PD-L1, a PD-1 receptor ligand. Interestingly, gene copy number of PD-L1 predicts response to such immunotherapy even though expression of PD-L1 protein is used to indicate administration of these therapies.

It has been reported that loss of function mutations in JAK1/2 can lead to resistance to anti-PD-1 therapy. See, e.g., Shin et al., Primary resistance to PD-1 blockade mediated by JAK1/2 mutations, Cancer Discov. 2017 February; 7(2): 188-201; Zaretsky et al., Mutations Associated with Acquired Resistance to PD-1 Blockade in Melanoma, N Engl J Med 2016; 375:819-829. We believe this is the first report that loss of JAK2 plays a role in immunotherapy response and examined whether gene copy number of JAK2 generally predicts response to immunotherapy. FIGS. 3N-3S show Kaplan-Meier plots as above, except that copy number of JAK2 was determined for various cancers: colorectal (FIG. 3N; p-value=0.001), neuroendocrine (FIG. 3O; p-value=0.019), lung (FIG. 3P; p-value=0.036), liver (FIG. 3Q; p-value=0.014), ovarian (FIG. 3R; p-value=0.016), or sarcoma (FIG. 3S; p-value=0.008). The separation was significant in all settings.

As shown in FIGS. 3N-3S, copy number of JAK2 alone was significantly correlated with response to immunotherapy in various cancers, although the effect size appeared to be reduced in lung (i.e., HR=0.861 in lung (FIG. 3P) versus HR<0.4 in colorectal, neuroendocrine, liver, ovarian and sarcoma). We then looked at whether copy number analysis of both PD-L1 and JAK2 would further improve the effect size in a highly curated set of 98 lung cancers. Results are shown using various survival metrics in FIGS. 3T-3V: time-on-treatment (FIG. 3T; TOT); time-to-next-treatment (FIG. 3U; TTNT); or overall survival (FIG. 3V; OS). A random forest model was used to set copy thresholds. In these figures, “R” refers to patients that responded to immunotherapy and “NR” refers to patients with lack of or less response. Separation was statistically significant using all metrics (p-value≤0.001) and HR was similar to that observed with head and neck cancer. CfFIG. 31, head and neck with HR=0.279 and FIG. 3V, lung with HR=0.266. These data indicate that copy number analysis of PD-L1 and JAK2 is a strong predictor of response to immunotherapy across cancer types.

Taken together, this Example shows that loss at 9p can be used to predict response to immunotherapy. Patients that did not show loss in this region had significantly greater survival after treatment with anti-PD1 immunotherapies. The effect was localized to chromosomal band 9p24. Analysis of copy number of PD-L1 along with other genes located in 9p24 such as JAK2 or PD-L2 showed strong predictive capabilities. A particularly strong statistical signal was observed when assessing copy number of PD-L1 and JAK2.

### Example 3: Selecting Treatment for a Cancer Patient

An oncologist treating a cancer patient desires to determine whether to treat the patient with checkpoint inhibitor immunotherapy. A biological sample comprising tumor cells and/or cell free nucleic acids from the patient is collected. A molecular profile is generated for the sample. Loss at chromosome 9p or portions thereof (e.g., band 9p24, or genes such as PD-L1, JAK2, PD-L2) are used to classify the molecular profile as indicative of likely response (benefit) or non-response (lack of benefit) to the immunotherapy. See Example 2. The classification is included in a report that also describes the molecular profiling that was performed. The report is provided to the oncologist. The oncologist uses the classification in the report to assist in determining a treatment regimen for the patient. If the classification is responder/benefiter, the oncologist may choose to treat that patient with checkpoint inhibitor immunotherapy. If the classification is non-responder, the oncologist may choose to treat the patient with alternate therapy, which may, at the discretion of the oncologist, comprise a combination of immunotherapy and chemotherapy.

### Other Embodiments

It is to be understood that while the invention has been described in conjunction with the detailed description thereof, the foregoing description is intended to illustrate and not limit the scope as described herein, which is defined by the scope of the appended claims. Other aspects, advantages, and modifications are within the scope of the following claims.

What is claimed is:

1. A method of treating a cancer in a subject, the method comprising:
(a) obtaining a biological sample comprising cells and/or cell free materials derived from the cancer in the subject;
(b) performing an assay to assess a copy number of chromosome 9 or a portion thereof in the biological sample; and
(c) administering a treatment for the cancer to the subject based on the assessment of step (b).

2. The method of claim 1, wherein the assessment in step (b) comprises determining a copy number of chromosome 9p or a portion thereof, wherein optionally the assay comprises at least one of sequencing, hybridization, amplification, next-generation sequencing, whole-genome sequencing (WGS), whole-exome sequencing (WES), whole-transcriptome sequencing (WTS), in situ hybridization (ISH), comparative genomic hybridization (CGH), high-resolution array comparative genomic hybridization (aCGH), microarray-based platforms, and PCR techniques.

3. The method of claim 1 or 2, wherein the portion of chromosome 9 comprises chromosome band 9p24 or a portion thereof.

4. The method of any one of claims 1-3, wherein the portion of chromosome 9 comprises one or more gene located in chromosome band 9p24.

5. The method of claim 4, wherein the one or more gene comprises DDX11L5, WASHC1, MIR1302-9HG, MIR1302-9, FAM138C, PGM5P3-AS1, PGM5P3, LINC01388, FOXD4, CBWD1, LOC105375942, LOC105375943, DOCK8, DOCK8-AS1, LOC105375945, LOC112268042, KANK1, RPL12P25, FAM217AP1, LOC105375947, RNU6-1327P, EIF1P1, LOC107987042, LOC105375949, DMRT1, DMRT3, RNU6-1073P, DMRT2, H3P29, LINC01230, RPS27AP14, LOC102723803, RNA5SP279, LOC105375951, LOC105375953, LOC105375952, SMARCA2, RNU2-25P, LOC107987043, RN7SL592P, LOC105375955, LOC101930053, LOC105375956, LOC101930048, VLDLR-AS1, VLDLR, LOC105375957, KCNV2, PUM3, GPS2P1, ATP5PDP2, CARM1P1, LINC01231, LOC105375959, RFX3, RFX3-AS1, LOC105375962, GLIS3, GLIS3-AS1, LOC105375964, LOC107986989, RNU6-694P, SLC1A1, SPATA6L, RPS6P11, PLPP6, CDC37L1-DT, CDC37L1, AK3, ECM1P1, RPS5P6, RCL1, KLF4P1, MIR101-2, HNRNPA1P41, JAK2, INSL6, CSNK1G2P1, PDSS1P1, MTND6P5, MTND5P36, MTND1P11, MTND2P36, MTCO1P11, MTCO2P11, MTATP6P11, MTCO3P11, MTND3P14, MTND4LP6, MTND4P14, MTND5P14, TCF3P1, LOC107987044, IGHEP2, INSL4, RLN2, HMGN2P31, RLN1, PLGRKT, RNF152P1, CD274, PDCD1LG2, RIC1, ERMP1, AK4P4, KIAA2026, MLANA, MIR4665, RANBP6, GTF3AP1, IL33, LOC107987046, SELENOTP1, TPD52L3, UHRF2, GLDC, RN7SL25P, RPL23AP57, RN7SL123P, RNF2P1, RPL35AP20, LINC02851, KDM4C, PRELID3BP11, SNRPEP2, ACTG1P14, LOC105375969, LOC105375970, LOC102723994, RPL4P5, PPIAP33, DMAC1, LOC105375971, PTPRD, RPL18AP11, RNU7-185P, PTPRD-AS1, or any useful combination thereof.

6. The method of claim 5, wherein the one or more genes comprises PD-L1, JAK2, or PD-L1 and JAK2, or wherein the one or more gene consists of PD-L1, JAK2, or PD-L1 and JAK2.

7. The method of any one of claims 1-6, further comprising predicting whether the subject will benefit or not benefit from administration of an immunotherapy.

8. The method of claim 7, wherein loss of copy number of chromosome 9 or the portion thereof indicates lack of benefit of the immunotherapy.

9. The method of claim 7, wherein the absence of loss of copy number of chromosome 9 or the portion thereof indicates potential response to the immunotherapy.

10. The method of claim 8 or 9, wherein the threshold for loss of copy number is determined using a statistical model, optionally wherein the statistical model is a machine learning model.

11. The method of any one of claims 7-10, wherein the immunotherapy comprises an immune checkpoint therapy.

12. The method of claim 11, wherein the immune checkpoint therapy comprises at least one of anti-PD-1 therapy, anti-PD-L1 therapy, anti-CTLA-4 therapy, ipilimumab, nivolumab, pembrolizumab, atezolizumab, avelumab, durvalumab, cemiplimab, and any combination thereof.

13. The method of any one of claims 7-12, wherein the subject has not previously been treated with an immunotherapy or the immunotherapy.

14. The method of any one of claims 1-13, wherein the cancer comprises a metastatic cancer, a recurrent cancer, or a combination thereof.

15. The method of any one of claims 1-14, wherein the subject has not previously been treated for the cancer.

16. The method of any one of claims 7-15, wherein the subject has a loss of copy number of chromosome 9 or the portion thereof and wherein the administered treatment for the cancer is a treatment that is different from the immunotherapy.

17. The method of claim 16, wherein the administered treatment for the cancer is a chemotherapy or a combination of immunotherapy and chemotherapy.

18. The method of any one of claims 7-15, wherein the subject does not have a loss of copy number of chromosome 9 or the portion thereof and wherein the administered treatment of the cancer is the immunotherapy.

19. The method of any one of claims 1-18, wherein progression free survival (PFS), disease free survival (DFS), or lifespan is extended by the administration of the treatment.

20. The method of any one of claims 1-19, wherein the biological sample comprises formalin-fixed paraffin-embedded (FFPE) tissue, fixed tissue, a core needle biopsy, a fine needle aspirate, unstained slides, fresh frozen (FF) tissue, formalin samples, tissue comprised in a solution that preserves nucleic acid or protein molecules, a fresh sample, a malignant fluid, a bodily fluid, a tumor sample, a tissue sample, or any combination thereof.

21. The method of any one of claims 1-20, wherein the cells and/or cell free materials derived from the cancer are from a solid tumor.

22. The method of any one of claims 1-21, wherein the biological sample comprises a bodily fluid, and optionally wherein the material derived from cancer cells comprises cell free nucleic acids.

23. The method of claim 22, wherein the bodily fluid comprises a malignant fluid, a pleural fluid, a peritoneal fluid, or any combination thereof.

24. The method of claim 22 or 23, wherein the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid, pre-ejaculatory fluid, female ejaculate, sweat, fecal matter, tears, cyst fluid, pleural fluid, peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyst cavity fluid, or umbilical cord blood.

25. The method of any one of claims 1-24, wherein the cancer comprises an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilms' tumor.

26. The method of any one of claims 1-24, wherein the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumor (GIST), glioblastoma, head and neck squamous carcinoma, leukemia, liver hepatocellular carcinoma, low grade glioma, lung bronchioloalveolar carcinoma (BAC), non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non-epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma.

27. The method of any one of claims 1-26, wherein the cancer comprises a head and neck cancer, neuroendocrine cancer, lung cancer, liver cancer, ovarian cancer, or sarcoma.

28. The method of any one of claims 1-27, wherein the cancer comprises breast cancer, bladder cancer, cervical cancer, colon cancer, head and neck cancer, Hodgkin lymphoma, liver cancer, lung cancer, renal cell cancer, melanoma, stomach cancer, rectal cancer, or any solid tumor that exhibits DNA replication errors, e.g., mutations, insertions, deletions, mismatch repair deficiency (MMRd), microsatellite instability (MSI-H), high tumor mutational burden (TMB), copy number variations (CNV).

29. The method of any one of claims 1-27, wherein the cancer comprises a head and neck cancer or a lung cancer.

30. A method of selecting a treatment for a subject who has a cancer, the method comprising:
(a) obtaining a biological sample comprising cells and/or cell free material derived from the cancer in the subject;
(b) performing an assay to assess a copy number of chromosome 9 or a portion thereof in the biological sample, optionally wherein the assay comprises at least one of sequencing, hybridization, amplification, next-generation sequencing, whole-genome sequencing (WGS), whole-exome sequencing (WES), whole-transcriptome sequencing (WTS), in situ hybridization (ISH), comparative genomic hybridization (CGH), high-resolution array comparative genomic hybridization (aCGH), microarray-based platforms, and PCR techniques; and
(c) selecting a treatment for the cancer to the subject based on the copy number of chromosome 9 or the portion thereof in (b).

31. The method of claim 30, wherein the portion of chromosome 9 comprises arm 9p, band 9p24, one or more gene located at 9p24, the PD-L1 gene, the JAK2 gene, the PD-L1 and JAK2 genes, or any useful combination thereof, optionally wherein the one or more gene comprises DDX11L5, WASHC1, MIR1302-9HG, MIR1302-9, FAM138C, PGM5P3-AS1, PGM5P3, LINC01388, FOXD4, CBWD1, LOC105375942, LOC105375943, DOCK8, DOCK8-AS1, LOC105375945, LOC112268042, KANK1, RPL12P25, FAM217AP1, LOC105375947, RNU6-1327P, EIF1P1, LOC107987042, LOC105375949, DMRT1, DMRT3, RNU6-1073P, DMRT2, H3P29, LINC01230, RPS27AP14, LOC102723803, RNA5SP279, LOC105375951, LOC105375953, LOC105375952, SMARCA2, RNU2-25P, LOC107987043, RN7SL592P, LOC105375955, LOC101930053, LOC105375956, LOC101930048, VLDLR-AS1, VLDLR, LOC105375957, KCNV2, PUM3, GPS2P1, ATP5PDP2, CARM1P1, LINC01231, LOC105375959, RFX3, RFX3-AS1, LOC105375962, GLIS3, GLIS3-AS1, LOC105375964, LOC107986989, RNU6-694P, SLC1A1, SPATA6L, RPS6P11, PLPP6, CDC37L1-DT, CDC37L1, AK3, ECM1P1, RPS5P6, RCL1, KLF4P1, MIR101-2, HNRNPA1P41, JAK2, INSL6, CSNK1G2P1, PDSS1P1, MTND6P5, MTND5P36, MTND1P11, MTND2P36, MTCO1P11, MTCO2P11, MTATP6P11, MTCO3P11, MTND3P14, MTND4LP6, MTND4P14, MTND5P14, TCF3P1, LOC107987044, IGHEP2, INSL4, RLN2, HMGN2P31, RLN1, PLGRKT, RNF152P1, CD274, PDCD1LG2, RIC1, ERMP1, AK4P4, KIAA2026, MLANA, MIR4665, RANBP6, GTF3AP1, IL33, LOC107987046, SELENOTP1, TPD52L3, UHRF2, GLDC, RN7SL25P, RPL23AP57, RN7SL123P, RNF2P1, RPL35AP20, LINC02851, KDM4C, PRELID3BP11, SNRPEP2, ACTG1P14, LOC105375969, LOC105375970, LOC102723994, RPL4P5, PPIAP33, DMAC1, LOC105375971, PTPRD, RPL18AP11, RNU7-185P, PTPRD-AS1, or any useful combination thereof.

32. The method of claim 30 or 31, further comprising preparing a molecular profile for the subject based on the copy number of chromosome 9 or the portion thereof.

33. The method of any one of claims 30-32, wherein the treatment comprises a checkpoint inhibitor therapy, e.g., anti-PD-1 therapy, anti-PD-L1 therapy, anti-CTLA-4 therapy, nivolumab, pembrolizumab, ipilimumab, atezolizumab, avelumab, durvalumab, or cemiplimab, a chemotherapy, or any useful combination thereof, based on the copy number.

34. The method of claim 33, further comprising administering the checkpoint inhibitor therapy to the subject when the subject is predicted to benefit from the therapy, and/or administering chemotherapy or chemotherapy in addition to the checkpoint inhibitor therapy when the subject is predicted to lack benefit from the therapy.

35. The method of any one of claims 30-34, wherein the cancer comprises a head and neck cancer, neuroendocrine cancer, lung cancer, liver cancer, ovarian cancer, or sarcoma; or breast cancer, bladder cancer, cervical cancer, colon cancer, head and neck cancer, Hodgkin lymphoma, liver cancer, lung cancer, renal cell cancer, melanoma, stomach cancer, rectal cancer, or any solid tumor that exhibits DNA replication errors, e.g., mutations, insertions, deletions, mismatch repair deficiency (MMRd), microsatellite instability (MSI-H), high tumor mutational burden (TMB), copy number variations (CNV).

36. A method of generating a molecular profiling report comprising preparing a report summarizing results of performing the method according to any one of claims 1-35.

37. The method of claim 36, wherein the report comprises any identified treatment of likely benefit and/or lack of benefit according to any one of claims 7-18 or 30-35.

38. The method of claim 36 or 37, wherein the report is computer generated; is a printed report or a computer file; or is accessible via a web portal.

39. A system comprising one or more computers and one or more storage media storing instructions that, when executed by the one or more computers, cause the one or more computers to perform operations in order to carry out the method of any one of claims 1-38.

40. A system for identifying a treatment for a cancer in a subject, the system comprising:
(a) at least one host server;
(b) at least one user interface for accessing the at least one host server to access and input data;
(c) at least one processor for processing the inputted data;
(d) at least one memory coupled to the processor for storing the processed data and instructions for:
(1) accessing results of analyzing the biological sample according to any one of claims 30-35; and
(2) determining likely benefit or lack of benefit of an immunotherapy according to any one of claims 30-33; and
(e) at least one display for displaying the likely benefit or lack of benefit of the immunotherapy for treating the cancer.

41. The system of claim 40, wherein the at least one display comprises a report comprising the results of analyzing the biological sample and the predicted likely benefit or lack of benefit for treatment of the cancer.
